(19) United States
(12) Patent Application Publication
(10) Pub. No.: US 2023/0212216 A1
(43) Pub. Date: Jul. 6, 2023

(54) SYNTHESIS OF LACTONE DERIVATIVES AND THEIR USE IN THE MODIFICATION OF PROTEINS

(71) Applicant: GENIE BIOTECH UK LTD., Frarnborough (GB)

(72) Inventors: Alexander Redfern MORRIS, Jersey (GB); Grigorij SUTOV, Jersey (GB); Karl Dietrich BRUNE, Jersey (GB)

(21) Appl. No.: 17/787,131

(22) PCT Filed: Dec. 18, 2020
(86) PCT No.: PCT/EP2020/087108

§ 371 Date: Jun. 17, 2022

(30) Foreign Application Priority Data
Dec. 20, 2019 (EP) 19218587.4

(51) Int. Cl. C07K 1/107; C07K 1/22; G01N 33/68; G01N 33/543; G01N 27/447; B01D 15/38; B01D 15/42

(52) U.S. Cl. CPC C07K 1/1077; C07K 1/22; G01N 33/6851; G01N 33/54386; G01N 27/447; B01D 15/3804; B01D 15/424; G01N 2440/10

(57) ABSTRACT

Site-specific modifications of proteins are desirable in biotechnological applications such as biopharmaceuticals, immunotherapy, vaccines, and are useful in chemical biology. Gluconoylation is a non-enzymatic, covalent, post-translational modification commonly observed on N-terminal His-Tags bearing proteins. We synthesized glucono-1,5-lactone derivatives, including azido variants for selective acylation. High yield acylation is achieved by simply mixing derivatives with target protein amidst diverse conditions of temperatures, aqueous buffers, excipients, or complex cell lysate.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is Revised_SEQUENCE_LISTING_390129_401USPC.txt. The text file is 20 KB, was created on Jan. 6, 2023, and is being submitted electronically via Patent Center.

### BACKGROUND

### Technical Field

The present disclosure relates to methods of synthesizing lactone derivatives as well as methods for chemically modifying, and purifying modified proteins. The methods of synthesizing lactone derivatives are encompassed in the field of carbohydrate chemistry and the method for modifying, and purifying modified proteins is encompassed in the field of protein chemistry.

### Description of the Related Art

Well-characterized protein bioconjugates are indispensable for chemical biology studies and biopharmaceutical applications. Traditional methods for obtaining bioconjugates typically target naturally-occurring chemical groups, such as the abundant amino (Lys, N-terminus) or carboxyl groups (Asp, Glu, C-terminus), or the less common sulfhydryl groups (Cys) (Hermanson 2013). Site-selective protein conjugation is desirable but technically challenging (Rosen and Francis 2017). Unless the targeted chemical group is uniquely present in a biomolecule, heterogeneous subpopulations of bioconjugates may be obtained. Heterogenous subpopulations are undesirable because they can display vastly different biological and pharmaceutical properties. Mandatory regulatory downstream mass spectrometric characterizations of bioconjugates is also complicated by heterogeneity, as the mass of the base protein is split into two or more values, even if the obtained heterogeneity should be biologically harmless (Kieran F. Geoghegan 2016).

Hence there is a need for methods that reduce heterogeneity in biopharmaceutical formulations (PEGylation etc.), antibody-drug conjugates (Jain et al. 2015), immunomodulatory conjugates and conjugate vaccines (Kanekiyo, Ellis, and King 2019), as well as for biomaterials (Pröschel et al. 2015).

Methods for natural amino acid residue modification are plentiful and have been reviewed in detail (deGruyter, Malins, and Baran 2017).

Unnatural amino acids with bioorthogonal reactivities may be incorporated into recombinant proteins by feeding the expression host modified amino-acids (Datta et al. 2002), the exploitation of Amber host suppression (L. Wang et al. 2001), genetic code expansion (Xie and Schultz 2006; Davis and Chin 2012; Lang and Chin 2014) or the use of Genetically recoded organism (Lajoie et al. 2013). A common drawback is that these approaches require expensive, chemically difficult to synthesize amino acid derivatives. Not only are special strains required but due to the promiscuity of tRNA synthases, reports of undesirable miss incorporation of unnatural amino acids into non-target positions, detectable by mass spectrometric approaches, have been recently added to the literature (Aerni et al. 2015; Gan and Fan 2017; Kunjapur et al. 2018).

Alternative approaches rely on the engineering of protein domains or enzymes. For example, Split-Inteins (Hirata et al. 1990), or Catcher/Tag technology (Bijan Zakeri and Howarth 2010) have been described. Recently, site-specific transglutaminases (Steffen et al. 2017), and peptide-peptide ligases such as Catcher/Tag-derived Spy- and SnoopLigase (Fierer, Veggiani, and Howarth 2014; Buldun et al. 2018), butelase (Nguyen et al. 2014; Cao et al. 2016), and engineered asparaginyl endopeptidases1 (Harris et al. 2015; R. Yang et al. 2017; Jackson et al. 2018) have been presented in addition to the traditional sortase approach (Mazmanian 1999). The common drawback of enzymatic and protein domain-based approaches is that high concentrations of linkage partners and/or large excess of ligation partner is required (e.g., sortases), ligating enzymes need to be removed after the conjugation, or that protein scars arise (e.g., SpyCatcher:SpyTag leaves a ˜100 aa scar, ˜11.5 kDa in Mw).

One particular strategy is the modification of proteins at their N-termini. Modifying N-termini appears attractive as there can only be one N-terminus per linear polypeptide sequence and more than 80% of all monomeric structures in the PDB have their N-terminus exposed (Jacob and Unger 2007). Importantly the pKa value of the N-terminal α-amino group is lower (pKa 7.6-8.0) than those of typical Lys side-chain ε-amines (pKa 10.5+1.1) and the N-terminus may therefore be targeted for selective, pH-controlled acyl- or alkylation (Grimsley, Scholtz, and Pace 2008).

However, most chemical N-terminal modification techniques can lead to off-site modifications, e.g., lysine acylation within the protein (see, for example, Martos-Maldonado et al. 2018)—thus a challenge in the field is to achieve N-terminal modification whilst minimizing off-site modification (Rosen and Francis 2017).

Most proteins that are fused to an N-terminal His6 tag for immobilized metal affinity chromatography (IMAC) purification actually contain the sequence MGSSHHHHHH, due to being cloned into the popular pET vector series for recombinant bacterial production in Escherichia coli. An NCBI protein blast yields >20,000 search results (exceeding server limit, likely more) for this particular sequence, likewise a lens.org protein blast against the US patent office database results in ˜20,000 individual patent families (grants ˜3,600, applications ˜16,500) utilising this sequence, confirming the widespread use of this purification tag. The particular tag sequence is utilized in ˜60% of crystallographic studies (Derewenda 2004).

Expression of an MGSSHHHHHH tagged protein in E. coli B-strains, such as the widely used BL21(DE3) or other B-strain derivatives, can lead to partial gluconoylation at the Met-cleaved (methionylaminopeptidase) alpha-amino group at the N-terminus of the recombinant protein (K. F. Geoghegan et al. 1999). Biologically derived gluconoylation is caused by a dead-end metabolite in B-strains—either gluconolactone or its phosphorylated derivative 6-phosphogluconolactone (Aon et al. 2008). The resulting 6-phosphogluconolactone would usually be metabolized by action of 6-phosphogluconolactonase (PGL) in wildtype E. coli, however this enzyme is lacking in E. coli B-strains due to random mutagenic strain engineering that was performed in order to render the B-isolate non-pathogenic (PGL was an off-target deletion and is not involved in pathogenicity) (Aon et al. 2008; Noll et al. 2013). Hence 6-phosphogluconolactone accumulates and is attacked by the alpha-amino group of exposed N-termini resulting in gluconoylated N-terminal residues. E. coli native phosphatases may hydrolyze the phosphate group before or after attack of the lactone to the N-terminus, typically yielding 3 distinct species resolvable by MS: the unmodified recombinant protein, the +178 Da gluconoylated—and the +258 Da phospho-gluconoylated species (K. F. Geoghegan et al. 1999).

### BRIEF SUMMARY

The present disclosure aims to synthesize azido-substituted lactone derivatives through a more atom-economical, chemically safer and greener route. Further, the present disclosure aims to use these derivatives to site-selectively modify the N-termini of peptides and proteins. The present disclosure aims to analyses and purify these site-selectively modified protein derivatives. In another aspect, the disclosure aims to enable storage of the protein derivatives.

### BRIEF DESCRIPTION OF THE SEVERAL VIEWS OF THE DRAWINGS

FIG. 1: Deconvoluted spectra of native and 6-azido-6-deoxy-D-glucono-1,5-lactone (6-AGDL) acylated (bottom) GSS-H₆-AffiEGFR protein. QTOF mass spectra of native (top) and 6-AGDL acylated (bottom) GSS-H₆-AffiEGFR in the m/z range of 14,300-15,100. SM(H+) indicates singly protonated starting material, SM-Ac (H+)-singly protonated, acetylated starting material, P1 (H+)-singly protonated reaction product containing one azido group, P2 (H+)-singly protonated reaction product containing two azido groups. The theoretical average m values are 14429 for SM, 14471 for SM-Ac, 14632 for the P1, 14635 for the P2, all [M].

FIG. 2: Two-step mono-functionalization of a model protein with 6-AGDL and metal-free click chemistry. (A) Schematic representation of the two-step mono-functionalization of GSS-H₆-AffiEGFR. (B) MALDI-TOF MS spectra showing subsequent acylation of GSS-H₆-AffiEGFR with 6-AGDL and BCN-amine. Top panel shows GSS-H₆-AffiEGFR prior 6-AGDL acylation, the middle panel shows mono-functionalized GSS-H₆-AffiEGFR after incubation with 6-AGDL, and the bottom panel shows the species after subsequent metal-free click chemistry-mediated conjugation of 6-AGDL:GSS-H₆-AffiEGFR with BCN-amine. Spectra were acquired in positive linear mode. SM(+2): GSS-H₆-AffiEGFR starting material, doubly charged, protonated; SM-Ac(+2)-GSS-H₆-AffiEGFR acetylated starting material, doubly charged, protonated; P1(+2): GSS-H₆-AffiEGFR product acylated with one 6-AGDL tag, doubly charged, protonated; P1(+2, 2×Na+): GSS-H₆-AffiEGFR product acylated with one 6-AGDL tag, doubly charged, sodiated; P1-BCN (+2): P1 conjugated with BCN-amine, doubly charged, protonated; P1-BCN (+2, 2×Na+): P1 conjugated with BCN-amine, doubly charged, sodiated.

FIG. 3: Reversibility of GDL:G-H₆-Beltide-1 in the presence of various CuSO₄ and EDTA concentrations at pH 7.5. GDL acylated G-H₆-Beltide-1 was produced as before, extra GDL was removed by C18 reverse phase purification and the acylated peptide (20 μM) was stored in 50 mM HEPES (NaOH) buffer supplemented with various concentrations of CuSO₄ and EDTA at 37° C. Change in acylation status was monitored by MALDI-TOF MS TIC and is reported as the relative m/z ratio of the acylated (m₁) and non-acylated species (m₂) ([m_{1/z}/(m_{1/z}+m_{2/z})]*100%).

FIG. 4: mP-AGE affords a high-resolution, low-tech solution for small molecular weight increases. GSS-H₆-AffiEGFR (50 μM) was incubated with 100 mM GDL or 100 mM 6-AGDL for 1 h at RT in 200 mM HEPES pH 7.5 followed by repeated spin-filtering into ultrapure water. Conjugation was followed by mixing to 1×SDS-PAGE loading buffer (pH 6.8), heating to 75° C. for 3 minutes and analysis on mP-AGE (0.1% w/v MBPA) with Coomassie staining. Mw band shifts are indicated by arrows for (1) the GDL- (theoretical Mw 14607 Da), and (2) the 6-AGDL-acylated (theoretical Mw 14632 Da) species. The acylated GSS-H₆-AffiEGFR protein has a theoretical Mw of 14429 Da. The drastic increase in retention time of the acylated protein in relation to the non-acylated protein is caused by the azido-gluconoylation and acrylamide cross-linked boronic acid derivative MBPA interaction.

FIG. 5: Visualization of 6-AGDL modification with mP-AGE. GSS-H₆-MBP, and G-H₆-flagellin (both 50 μM) were incubated with 100 mM 6-AGDL for 1 h at RT in 200 mM HEPES (NaOH) pH 7.5. The reactions were desalted by repeated spin-filtering into ultrapure water. The samples were prepared to 1×SDS-PAGE loading buffer (pH 6.8), heated to 75° C. for 3 minutes and analyzed on (A) a standard 14% SDS-polyacrylamide gel, or (B) 14% polyacrylamide mP-AGE (the same gel preparation supplemented with 0.09% w/v MBPA). The gels were stained with Coomassie. The apparent Mw band shifts are indicated by arrows. The drastic increase in retention time of the acylated in relation to the non-acylaled protein is caused by the azido-gluconoylation and acrylamide-cross-linked boronic acid derivative MBPA interaction.

FIG. 6: 6-AGDL modified material was resolved from unmodified and acetylated material by online column chromatography. The analytes were continuously detected at 280 nm. Whereas the native protein sample eluted in a single peak (top row, G-H₆-ΔN1SpyCatcher), the 6-AGDL treated sample mixture was resolved as two peaks (bottom row)—one peak with the retention time of the unmodified material and a new, later eluting peak (acylated as 6-AGDL:G-H6-ΔN1SpyCatcher). Peak fractions were collected manually for each peak, acidified, desalted (ZipTip C18) and subjected to MALDI-TOF MS intact mass analysis in linear positive mode as before. The material collected from the earlier eluting peak was indistinguishable from the untreated starting material by mass, displaying unmodified sample and some evidence of acetylated (possibly N-terminally blocked G-H₆-ΔN1 SpyCatcher protein), whereas the later eluting peak only showed the expected mass for a singly 6-AGDL modified species of G-H₆-ΔN1SpyCatcher. No unmodified starting material was identified in the mass spectrum of the later eluting peak.

FIG. 7: 2D ¹H-¹³C Heteronuclear Single Quantum Coherence (HSQC) NMR spectrum of 6-azido-6-deoxy-glucono-1,5-lactone for the C2-C6 region. Correlation signals in DMSO-d6 are boxed, whereas signals in MeOD-d4 are circled. Likely 6-azido-6-deoxy-glucono-1,4-lactone signals in DMSO-d6 are enclosed with triangles. ¹H signals are plotted on the horizontal axis, whereas ¹³C signals are plotted on the vertical axis. Dashed lines represent 1D ¹H and 1D ¹³C NMR signals in MeOD reported by Chaveriat et al. 2006.

FIG. 8: 2D ¹H-¹³C Heteronuclear Single Quantum Coherence (HSQC) NMR spectrum of 6-azido-6-deoxy-glucono-1,5-lactone for the C2-C6 region in D₂O. ¹H signals are plotted on the horizontal axis, whereas ¹³C signals are plotted on the vertical axis.

FIG. 9: SDS-PAGE indicates that VLPs could be successfully conjugated to the receptor binding domain (RBD) of SARS-CoV-2. VLP=Qbeta VLP activated with NHS-PEG8-BCN. RBD C−=RBD. RBD N3=RBD activated with 6-AGDL.

FIG. 10: Anti-RBD ELISA results. A) Serum samples were tested for the presence of antibodies that react to RBD after the first administration. B) Serum samples were tested for the presence of antibodies that react to RBD after the mice received a booster shot. RBD=RBD. RBD:GBT=RBD activated with 6-AGDL. RBD:GBT:Qbeta=RBD conjugated to Qbeta VLP using 6-AGDL and NHS-PEG8-BCN.

FIG. 11: Live virus neutralization assay using SARS-CoV-2 as a challenge. Serum samples were tested for their ability to neutralize viral infection and/or replication. Group 3=Qbeta VLP. Group 5=RBD produced in HEK293 cells. Group 6=RBD produced in HEK293 cells conjugated to 6-AGDL. Group 9=RBD produced in Pichia pastoris conjugated to Qbeta VLP using 6-AGDL and NHS-PEG8-BCN. Group 10=RBD produced in HEK293 cells conjugated to Qbeta VLP using 6-AGDL and NHS-PEG8-BCN.

### DETAILED DESCRIPTION

The present disclosure was conceived by the idea that derivatives of six-membered lactones could be used to site-specifically modify proteins with chemical handles. As far as the inventors of the present application are aware, such an approach has never been attempted in the prior art.

The inventors of the present application had to overcome several hurdles to reliably modify proteins with an azido-derivative of gluconolactone. One of these challenges was finding a suitable route for synthesizing pure compositions of 6-azido-6-deoxy-D-glucono-1,5-lactone.

Previous reported attempts to synthesize 6-azido-6-deoxy-D-glucono-1,5-lactone relied on bromine oxidation (Hanessian 1966; 1969; Kefurt et al. 1979; Chaveriat et al. 2006).

In explorative experiments it was observed that the azido-compound(s) obtained by aqueous bromine oxidation of 6-azido-6-deoxy-glucose according to Hanessian 1969 resulted in very little protein substrate being acylated in the hand of the present inventors, whereas reaction with commercial non-azido glucono-1,5-lactone yielded near complete conversion under the same conditions.

It is known that the aqueous bromine oxidation product of glucose, glucono-1,5-lactone, can be in a complex equilibrium with other species. Due to hydrolysis and isomerization of the 1,5-lactone, the reaction mixture assumes a complex equilibrium between open-chain gluconic acid, the glucono-1,5-lactone and glucono-1,4-lactone (Pocker and Green 1973). This equilibrium can be further complicated by the presence of cations, which can give rise to various corresponding acid salts. It may be assumed that the equilibrium of the oxidation product(s) of 6-azido-6-deoxy-glucose may be equally complex.

In exploratory experiments, the small but detectable amount of peptide acylation observed upon contacting the model protein with the bromine oxidation product(s) of 6-azido-6-deoxy-glucose may be explained by the synthesis of (i) minor amounts of the reactive 1,5-lactone, whereas the major product(s), e.g., the 1,4-lactone and/or the free acid may have reactivity with the peptide substrate. Alternatively (ii), bromine oxidation of the azido-sugar yields no 1,5-lactone and the product(s), such as the 1,4-lactone or the free acid, react—albeit poorly—with the peptide substrate either directly and/or via isomerization in situ to very small amounts of 1,5-lactone. In any case, aqueous bromine oxidation in our hands did not allow practically relevant production of suitable acylating reagent.

The free acid or its anion would not be expected to be reactive towards primary amines. It is known that 1,4-lactones can exhibit a markedly different reactivity compared to their 1,5-isomers. For example, it is known that the undecorated, non-azido glucono-1,4-lactone is a much weaker electrophile towards hydroxylamine compared to the 1,5-lactone (Miclet 2001). The 1,4-lactone also does not spontaneously hydrolyze in aqueous solutions at lower temperatures (5° C.), whereas the 1,5-form does, again demonstrating different chemical properties (Miclet 2001). It is known that six-membered 1,5-lactones can be selectively and completely reduced with a SmI₂—H₂O reducing system in a molecule that contains both a five- and six-membered lactone ring systems, whereas the five-membered 1,4-lactone system is left intact (Duffy, Matsubara, and Procter 2008). Again, this demonstrates that 1,4- and 1,5-lactones can exhibit different chemical reactivities.

After failing to obtain suitable acylating reagent with bromine oxidation, we re-evaluated the literature evidence for the structure of the major oxidation product of 6-azido-6-deoxy-glucose, claimed to be 6-azido-6-deoxy-glucono-1,5-lactone.

Hanessian 1969 reportedly relied on the infrared (IR) spectrum of the carbonyl to assign the six-membered ring of the product of bromine oxidation of the azido-glucopyranose as produced by acidic cleavage from the azidated methylpyranoside. However, relying solely on IR to assign the ring-size may be misleading. In particular, it is known that 1,4-lactones do not always strictly adhere to the IR spectroscopy guidelines developed by Barker and colleagues, i.e., 22 out of 24 aldono-1,4-lactones showed a band at 5.59 to 5.67 μm (1790 to 1765 cm⁻¹), whereas all 11 tested aldono-1,5-lactones showed a band at 5.68 to 5.79 μm (1760 to 1726 cm⁻¹) (Tipson 1968). Given that Hanessian 1969 obtained a value of 1732 cm⁻¹ for the six-membered model compound D-glucono-1,5-lactone, 1749 cm⁻¹ for the five-membered model compound D-galactono-1,4-lactone, and 1725 cm⁻¹ for the reaction product of 6-azido-6-deoxy-D-glucose oxidation, perhaps unsurprisingly Hanessian 1969 assigned the oxidation product as the 6-azido-6-deoxy-glucono-1,5-lactone. Confusingly however, Barker's trends would have suggested that the D-galactono-1,4-lactone compound should have been classified as an 1,5-lactone (Barker et al. 1958). As such, D-galactono-1,4-lactone is an example for a lactone for which the IR spectrum does not adhere Barker's rule.

Hanessian 1969 also reports bromine oxidation of 6-azido-6-deoxy-D-galactose, which according to infrared spectroscopy was analogously assigned as the 6-azido-6-deoxy-D-galactono-1,5-lactone with 1735 cm⁻¹. However, isolation of D-galactono-1,5-lactone by aqueous bromine oxidation of D-galactose has not been achieved and is deemed impossible (Bierenstiel 2004). Presumably the 1,5-lactone quickly equilibrates towards the galactono-1,4-lactone and/or hydrolyses to the corresponding acid (Hudson and Isbell 1929; Bierenstiel and Schlaf 2004). Hence, one may question if Hanessian 1969 really obtained 6-azido-6-deoxy-D-galactono-1,5-lactone, and likewise 6-azido-6-deoxy-D-glucono-1,5-lactone. More confusingly, Hanessian reports the synthesis of 6-azido-6-deoxy-D-galactono-1,4-lactone in an earlier communication, however neither the synthetic route nor the compound characteristics other than the m.p. of 138-140° C. were disclosed (Hanessian 1966). Hanessian's 1969 communication states with a note that the product of the 1969 is identical to the product previously described in 1966, suggesting that bromine oxidation was used in 1966. However, neither the reported ring sizes nor the melting points of the previous communication match (1,4-lactone with 138-140° C. in 1966 vs. 1,5-lactone with 129-130° C. in 1969).

The complexity is further compounded by a report from another group: Bromine oxidation of 6-azido-6-deoxy-D-glucose, akin to Hanessian's 1969 route, resulted in a compound with comparable m.p. of Hanessian's 1969 compound but with an even lower carbonyl wave-number value of 1716 cm⁻¹, prompting Kefurt to assign the 6-azido-6-deoxy-glucono-1,5-lactone (Kefurt et al. 1979). Kefurt and co-workers also isolated an additional lower melting compound (114-118° C.) with identical IR spectrum, which they also assign as 6-azido-6-deoxy-D-glucono-1,5-lactone. Kefurt et al. speculate that the two differently melting compounds are crystalline modifications of the same 1,5-lactone.

In 2006, Chaveriat et al. performed bromine oxidation of the azido-glucose and report 1D NMR data for the first time obtaining a yellow oil instead of previously crystalline substance (Chaveriat et al. 2006).

1D ¹H and ¹³C NMR spectra were obtained in MeOD. The carbonyl shift of C1 and C6 in the absence of 2D NMR has been used by many chemists to assign the ring-size for basic and even substituted hexonolactones. The C1 shifts typically follow the numerical trend hexonic acid>1,4-lactone>1,5-lactone, with 1,5-lactones typically exhibiting a shift of ˜172-174.6 ppm, the 1,4-lactones displaying a shift of ˜175.5-177.9, and the free acid showing with shift value of >178 ppm in DMSO-d6 (typically lower values) and D₂O (typically higher values) (Walaszek and Horton 1982; Walaszek, Horton, and Ekiel 1982; Bierenstiel and Schlaf 2004). These relative C1 shifts can be also observed for C6-substituted lactones and acids, such as 6-phospho-substituted glucono-1,5- and 1,4-lactones and the gluconic acid/gluconate (Miclet et al. 2001; Moreno et al. 2017; Sadet et al. 2018). Chaveriats C1 value (173.2, MeOD) is thus closer to a 1,5-structure. C6 values of 1,5-lactones are also found upfield when compared to the corresponding 1,4-lactones or hexonic acids shifts (Walaszek and Horton 1982; Walaszek, Horton, and Ekiel 1982). Hence Chaveriats C6 value (53.4, MeOD) is also closer to a 1,5-structure, but strikingly lower than other previously recorded non-substituted hexonolactones. As we show later in this communication, azidation of C6 induces upfield shift of C6 values, which may make ring-size assignment of azidated hexono-lactones based on 1D ¹³C NMR alone more difficult, if not impossible without a 2D NMR reference.

In summary, prior art did not explicatively indicate nor suggest that aqueous bromine oxidation would necessarily result in an unsuitable compound for the present disclosure, as 1) bromine oxidation of D-glucose results in the easily obtainable crystalline 1,5-lactone (Isbell and Frusch 1933), 2) IR spectroscopy of the azido-lactone was in good agreement with Barker 1958 for a 1,5-lactone, 3) 1D ¹³C NMR spectroscopy was in agreement with literature for a 1,5-lactone, 4) and all compounds exhibited the expected reactivates for a lactone and were successfully used for the application explored in the respective communication. Noteworthy, all previous reports also passed scrutinizing academic peer-review.

Hence the present inventors had to first identify and then overcome a non-obvious hurdle in the prior art to enable the synthesis of 6-azido-6-deoxy-D-glucono-1,5-lactone for use in protein acylating reactions.

Only when faced with the results of the failed exploratory acylation experiments after bromine oxidation, together with the expected complex aqueous equilibrium, the non-definitive nature of IR and 1D ¹³C NMR analysis, we considered the possibility that the proposed 6-azido-6-deoxy-D-glucono-1,5-lactone may not necessarily be the correct or only structure for the bromine oxidation product and explored alternative synthesis routes.

Thus, the present disclosure provides a method for synthesizing a handle-substituted carbohydrate lactone comprising contacting a handle-substituted aldose with a catalyst under aprotic conditions in the presence of a hydrogen acceptor.

Once pure 6-azido-6-deoxy-D-glucono-1,5-lactone was obtained, the compound was found to efficiently and site-specifically acylate proteins.

Thus, the present disclosure also provides a composition comprising an N-terminally acylated protein, wherein the N-terminally acylated protein comprises formula (VII), formula (VIII) or formula (IX):

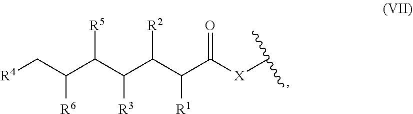

wherein:
(i) R¹, R², R³, R⁵ and R⁶ are each independently hydrogen, a hydroxyl, a methyl, or a handle,
(ii) R⁴ is hydrogen, a hydroxyl, a methyl, a handle, or a carboxyl,
(iii) at least one of R¹, R², R³ and R⁴ is a handle,
(iv) one or none of R¹, R², R³ and R⁴ is a methyl; and
(v) X comprises the N-terminal amino acid residue of the protein, wherein, optionally, the N-terminal amino acid residue is a Gly, Ala, Ser or His residue;

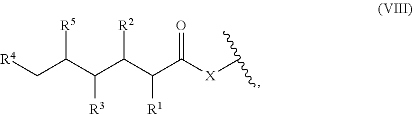

wherein:
(i) R¹, R², R³ and R⁵ are each independently hydrogen, a hydroxyl, a methyl, or a handle,
(ii) R⁴ is hydrogen, a hydroxyl, a methyl, a handle, a carboxyl, an ester or an amide,
(iii) at least one of R¹, R², R³ and R⁴ is a handle,
(iv) one or none of R¹, R², R³ and R⁴ is a methyl; and
(v) X comprises the N-terminal amino acid residue of the protein, wherein, optionally, the N-terminal amino acid residue is a Gly, Ala, Ser or His residue;

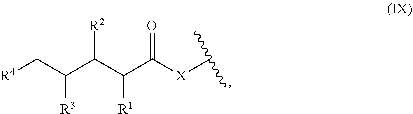

wherein:
(i) R¹, R² and R³ are each independently hydrogen, a hydroxyl, a methyl, or a handle,
(ii) R⁴ is a hydroxyl,
(iii) at least one of R¹, R² and R³ is a handle,
(iv) one or none of R¹, R² and R³ is a methyl; and
(v) X comprises the N-terminal amino acid residue of the protein, wherein, optionally, the N-terminal amino acid residue is a Gly, Ala, Ser or His residue.

The present disclosure also provides a composition comprising an N-terminally conjugated protein, wherein the N-terminally conjugated protein is obtained or obtainable by reacting the N-terminally acylated protein of the present disclosure with a compound comprising a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene).

Further, the present disclosure provides a method for site-specifically modifying a protein comprising contacting a protein with a handle-substituted carbohydrate lactone.

The present disclosure also provides the use of a diol-ester forming agent, and/or metal cation for modulating the hydrolysis rate of an acylated protein.

The present disclosure also provides a method for identifying an acylated protein comprising running a sample suspected of containing an acylated protein on a diol-interacting, boron-containing acrylamide gel, wherein the acylated protein is the acylated protein of the present disclosure, wherein, optionally, the diol-interacting, boron-containing acrylamide gel is a methacrylamido phenylboronate acrylamide gel.

Further, the present disclosure provides a method for purifying an acylated protein comprising: (1) binding a sample suspected of comprising the acylated protein onto a solid support comprising an immobilized diol-ester forming agent; and (2) eluting the protein.

The present disclosure also provides a kit comprising a protein and a handle-substituted carbohydrate lactone.

### DETAILED DESCRIPTION

### Definitions

The term “acylated protein” refers to any protein wherein an amino group of the protein has been chemically modified by a carbohydrate lactone. When the term “double acylated” is used in the present application, this refers to a protein that has been chemically modified at two sites with carbohydrate lactone. For example, the free amine group at the N-terminus as well as the free amine group of a lysine side-chain may have been acylated.

The term “adduct” as used in the present application refers to a product that is a direct addition of two or more distinct molecules, resulting in a single reaction product containing almost all atoms of all components.

The term “aprotic conditions” as used in the present application refers to any reaction conditions wherein the reaction is performed in one or more aprotic solvents. Aprotic solvents contain no hydrogen atoms connected directly to an electronegative atom and are not capable of hydrogen bonding. Exemplary aprotic solvents include cyclohexanone, dimethylsulfoxide, tetrahydrofuran, acetone, dimethylformamide and acetonitrile.

The term “azido-substituted pyranose” as used in the present application refers to a compound comprising 5-10 carbon atoms, hydrogen and oxygen, wherein 5 of the carbon atoms form a tetrahydropyrane ring and at least one of the carbon atoms is bonded to an azide group.

The term “azido-substituted six-membered 1,5-carbohydrate-lactone” as used in the present application refers to a saturated six-membered lactone ring comprising 5-10 carbon atoms, hydrogen and oxygen wherein one or more of the carbons at positions 2, 3, 4, 5, 6, or 7 are bonded to an azide group.

The term “boronic acid” as used in the present application refers to a compound related to boric acid in which one of the three hydroxyl groups is replaced by an alkyl or aryl group. It is understood that other diol-ester forming reagents, such as 2-, and 4-formylphenylboronic acid, benzoboroxoles or benzoxaboroles, Wulff-type boronates, 4-(methylcarbamoyl)-phenylboronic acid, or (2, or 4-[(dimethylamino)methyl]phenyl)boronic acid may be employed as well. It is also understood that synthetically more accessible boronic esters and derivatives can be used as well. This is because under aqueous conditions these esters hydrolyze to the boronic acid and become free to complex with diols of interest (Eising et al. 2016).

The term “contacting” as used in the present application refers to any action wherein two or more compounds are brought into sufficient proximity to one another to allow a reaction to occur. This includes, for example, mixing two or more components in a solution and incubating the mixture under conditions which allow the reaction to progress.

As used herein, the term “effective amount” of an agent, e.g., a therapeutic agent such as an antibody, is that amount sufficient to effect beneficial or desired results, for example, clinical results, and, as such, an “effective amount” depends upon the context in which it is being applied. The term “effective amount” can be used interchangeably with “effective dose,” “therapeutically effective amount,” or “therapeutically effective dose.”

The term “handle” refers to a chemical moiety which provides a protein with a non-native chemical functionality. Exemplary handles include Isothiocyanates, Isocyanates, Acyl azides, NHS ester (NHS, and -sulfo), in situ NHS ester (e.g., —COOH functional group), Hydroxybenzotriazole (HOBt) ester, Sulfonyl chloride, -flouride, Sulfonyl ester (e.g., tosyl, mesyl, trifyl, tresyl esters, etc.), Aldehydes, ketones, dicarbonyls (i.e., phenylglyoxal deriv.), Aldehyde, Aldehyde, Aldehyde, Amine, Epoxides, Carbonates (e.g., succinimidyl carbonate), Cyclic imidocarbonate, NHS carbonate, N,N′-disuccinimidyl carbonate (DSC), Cyanate ester, Carbamates (e.g., imidazole carbamate), Acyl imidazole, NHS carbamate, Aryl halides (e.g., fluorobenzene derivatives), Haloacetyl or alkyl halide, Imidoesters (imidates), Carboxylate, Acid anhydride, Fluorophenyl esters (pentafluorophenyl/PFP, tetrafluorophenyl/TFP, sulfo-tetrafluorophenyl/STP esters), Hydroxymethyl phosphines [Tris(hydroxymethyl)phosphine/THP or beta-[tris(hydroxymethyl)phospino]proprionic acid/THPP], Cyclic hemiacetal, Azlactones, Activated double bond, Methylpyridinium ether, 6-sulfo-cytosine derivative, activated halogens, such as haloacetyl derivatives (e.g., iodoacetyl), benzyl halides, and alkyl halides (N- and S-mustards) derivatives, Activated double bond, Vinyl sulfone, Maleimide, Aziridine, Aryl halide, Acryloyl derivatives (acrylic and methacrylic acid derivatives), Disulfides (e.g., protected thiol, such as, pyridyl disulfide deriv., 5-thiol-2-nitrobenzoic acid, TNB-thiol—obtainable by treating a sulfhyrdyl with Ellman's reagent), Thiols (free), Thioesters, such as phenylthioester (on C-terminal peptides for native chemical ligation), Cysteine attached via C-terminus (to enable NCL), Cisplatin derivatives, 2-cyanobenzothiazole, Epoxides, Diazoalkanes and diazoacetyl compounds, Epoxide, Isocyanate, Aryl azides (i.e., phenyl azide, perfluorinated phenyl azide), Benzophenone, Anthraquinones, Diazo compounds (i.e., diazotrifluoropropionate, diazopyruvate), Diazirene derivatives, Psoralen compounds, Halogenated phenyl azide, Alkenes, Dienes, Furan, Nitrones (Ning et al. 2010), Tetrazines (Li et al. 2010; Stockmann et al. 2011), nitrile oxides, diazoalkanes, syndones, quadricyclanes, Boronic acid (e.g., phenyl boronic acid, and phenyldiboronic acid), BCN, BCN, 1,3-Dithiolium-4-olates (Kumar et al. 2019), phosphines, e.g., triphenyl phosphine with electrophilic trap (Staudinger ligation), phosphine derivatives (bi- or tri-phenyl aryl ester, or acyl imidazole for traceless Staudinger ligation), alkene, alkyne (CuAAC), strained alkynes (SPAAC), OCT, MOFO, DIFO, DIFO2, DIFO3, DIMAC, DIBO, DIBAC (ADIBO), BARAC, BCN, Sondheimer diyene, TMDIBO, S-DIBO, COMBO, PYRROC, TMTH, DIFBO, ALO, isonitriles, thioalkyne (Destito et al. 2017), keto-DIBO, olefins, strained olefins, trifluoromethyl-substituted oxanorbornadienes (van Berkel et al. 2007), Biotin and it's derivatives, boronic acid, p-boronophenylalanine, strained dialkyne, Sondheimer dialkyne.

The term “hydrogen acceptor” as used in the present application refers to any molecule that can accept hydrogen in an oxidation-reduction reaction. Compounds comprising a ketone, carbonyl, alkene, alkyne and/or imine group are generally considered to be suitable hydrogen acceptors. Examples of hydrogen acceptors include acetone, butanone (methyl ethyl ketone), but-3-en-2-one, 3-methylbutan-2-one, 2-pentanone, 3-pentanone, 1-penten-3-one, 3,3-dimethyl-2-butanone, 2-methylpentan-3-one, 3-methyl-2-pentanone, 4-methylpentan-2-one (methyl isobutyl ketone), 3-penten-2-one, 3-hexanone (acetylacetone), 2-hexanone, 5-hexen-2-one, 4-methyl-3-penten-2-one, dibenzylideneacetone, cyclopentanone, 3-methyl-3-penten-2-one, 5-methylhexan-3-one, 4-methyl-2-hexanone, 2-heptanone (methyl n-amyl ketone), 4-heptanone, cyclohexanone, 4-octanone, diisobutyl ketone, diacetone alcohol (4-hydroxy-4-methylpentan-2-one), 3-octanone, 5-methylhexan-3-one, 2-octanone, oct-1-en-3-one, 5-methyl-2-octanone, cycloheptanone, ethyl acetoacetate (ethyl 3-oxobutanoate), 2-nonanone, 3-nonanone, 4-nonanone, 5-nonanone, 1-methylpyrrolidin-2-one, acetophenone, isophorone (3,5,5-trimethylcyclohex-2-en-1-one), benzylideneacetone, 2-decanone, 5-decanone, 3-phenyl-5-hexen-2-one, pentane-2,4-dione (acetylacetone), butanedione (butane-2,3-dione), benzil (1,2-diphenylethane-1,2-dione), diphenylacetylene, 1-hexyne, cyclohexene, pentanal, 1-methylcyclohexene, 1-octene, 2,4-dimethyl-3-pentanone, 4-octyne, styrene, diphenylacetylene, benzaldehyde, trans-stilbene, benzophenone, 1,3-diphenyl-2-propanone, anthracene, and 2-pentene. Other suitable hydrogen acceptors are known in the art (Conley et al. 2010). The hydrogen acceptors disclosed in Conley et al. 2010 are incorporated herein by reference and are encompassed by the term “hydrogen acceptor.”

The terms “individual,” “patient” or “subject” are used interchangeably in the present application to designate a human being and are not meant to be limiting in any way. The “individual,” “patient” or “subject” can be of any age, sex and physical condition. The term “animal,” as used in the present application, refers to any multicellular eukaryotic heterotroph which is not a human. In a preferred embodiment, the animal is selected from a group consisting of cats, dogs, pigs, ferrets, rabbits, gerbils, hamsters, guinea pigs, horses, rats, mice, cows, sheep, goats, alpacas, camels, donkeys, llamas, yaks, giraffes, elephants, meerkats, lemurs, lions, tigers, kangaroos, koalas, bats, monkeys, chimpanzees, gorillas, bears, dugongs, manatees, seals and rhinoceroses.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers. Linkers may include any of those taught in, for example, WO 2014/10628.

The term “N-terminal amino acid residue” refers to an amino acid residue that comprises a free amine group attached to its alpha carbon and is found at the end of the protein.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present disclosure, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington: The Science and Practice of Pharmacy 22^{nd} edition, Pharmaceutical press (2012), ISBN-13: 9780857110626 may also be included.

The term “protein” is used interchangeably with the term “peptide” in the present application. Both terms, as used in the present application, refer to molecules comprising one or more chains of amino acid residues. Examples include Catalytic proteins, such as Enzymes, Enzymes regulators, Protein Kinases/-Phosphatases, Proteases; Biomarkers, such as Blood Group Markers, Lysosomal Markers, Nuclear Markers, Cluster of differentiation molecules; Proteins suspected of causing disease, such as APP/B-Amyloid, Tau, Apolipoproteins; Transport proteins, such as Apolipoproteins, Ribosomal proteins; Affinity molecules, such as Antibodies (Abs), Fc-modified Abs, appended Abs, CH3 fusion proteins, Nanobodies, Affibodies, single-chain variable fragment (scFv), tandem-affinity fragment variable (taFv), miniantibodies, fragments of Abs (FABs), Fab₂, ImmTacs, Fab fusions, designed ankyrin repeat proteins (DARPins), Nanofitins, Affitins, Affilins, alpahReps, i-bodies, Repebodies, Anticalins, Atrimers, Avimers, Bicylic peptides, Centyrin, Cys-knots, Knottins, Monobodies, Adnectins, Fynomers, Kunitz domains, OBodies, bispecific Abs and Abs fragments, trispecifics Abs, Abs-fusions, beta-hairpin mimetics, diabody, small immunoprotein (SIPS), Armadillo repeat protein based scaffolds, Adhesion Molecules, Siglec Molecules; Signaling molecules such as Fc Receptors, Soluble Receptors; Antigens, such as extracellular antigens, intracellular antigens, extracellular membrane proteins, parasitic antigens, bacterial antigens, viral antigens, MHC Antigens, MHC tetramer, MHC monomer, self-antigens, self-neo-antigens, allergens, carrier proteins for conjugate vaccines, fusion proteins with multiple inserted epitopes, fusion proteins with immunomodulatory sequences, fusion proteins with cell-penetrating peptide sequence(s), fusion proteins of antigens and multimerization domains; Immunomodulatory proteins, such as Cytokines, GM-CSF, Chemokine Receptors, Immune Checkpoint Receptors, Innate Immune Signaling, T-cell receptors, Toll-like Receptors, Toll-like Ligands; Growth modulating proteins, such as Growth Factors, Neurotrophic Factors, Postsynaptic Proteins, Steroid Receptors/Nuclear, peptide hormones, Protein hormones; Movement associated molecules, such as Chemokines, Flagella; Viruses, such as Bacteriophages, Viruses, Virus-like particles; Nanoparticles, such as peptide-based nanoparticles, protein-based nanoparticles, outer-membrane vesicles, vesicles; Affinity tag peptide or protein, such as Gly-His-Tag; Covalent bond-forming proteins, such as Catcher/Tag split proteins, SpyCatcher and its variants, Gly-terminated SpyTag, Gly-terminated SpyCatcher, Split-intein and other (split)-proteins, such as G-protein coupled receptors, Voltage-gated ion channels, Plasma proteins, Transcription factors, Coagulation factors, Fusion proteins, Phosphoproteins, Lipoproteins, polymerases, ligases, hydrolases, nucleases, oxidoreductases, transferases, lyases, isomerases, translocases, reductases, clustered regularly interspaced short palindromic repeats (CRISPR) associated proteins, and Glycoproteins. Other examples include Lepirudin, Cetuximab, Dornase alfa, Denileukin diftitox, Etanercept, Bivalirudin, Leuprolide, Peginterferon alfa-2a, Alteplase, Interferon alfa-n1, Darbepoetin alfa, Reteplase, Epoetin alfa, Salmon Calcitonin, Interferon alfa-n3, Pegfilgrastim, Sargramostim, Secretin, Peginterferon alfa-2b, Asparaginase, Thyrotropin Alfa, Antihemophilic Factor, Anakinra, Gramicidin D, Intravenous Immunoglobulin, Anistreplase, Insulin Regular, Tenecteplase, Menotropins, Interferon gamma-1b, Interferon Alfa-2a, Recombinant, Coagulation factor VIIa, Oprelvekin, Palifermin, Glucagon recombinant, Aldesleukin, Botulinum Toxin Type B, Omalizumab, Lutropin alfa, Insulin Lispro, Insulin Glargine, Collagenase, Rasburicase, Adalimumab, Imiglucerase, Abciximab, Alpha-1-proteinase inhibitor, Pegaspargase, Interferon beta-1a, Pegademase bovine, Human Serum Albumin, Eptifibatide, Serum albumin iodonated, Infliximab, Follitropin beta, Vasopressin, Interferon beta-1b, Interferon alfacon-1, Hyaluronidase, Insulin, porcine, Trastuzumab, Rituximab, Basiliximab, Muromonab, Digoxin Immune Fab (Ovine), Ibritumomab, Daptomycin, Tositumomab, Pegvisomant, Botulinum Toxin Type A, Pancrelipase, Streptokinase, Alemtuzumab, Alglucerase, Capromab, Laronidase, Urofollitropin, Efalizumab, Serum albumin, Choriogonadotropin alfa, Antithymocyte globulin, Filgrastim, Coagulation factor ix, Becaplermin, Agalsidase beta, Interferon alfa-2b, Oxytocin, Enfuvirtide, Palivizumab, Daclizumab, Bevacizumab, Arcitumomab, Eculizumab, Panitumumab, Ranibizumab, Idursulfase, Alglucosidase alfa, Exenatide, Mecasermin, Pramlintide, Galsulfase, Abatacept, Cosyntropin, Corticotropin, Insulin aspart, Insulin detemir, Insulin glulisine, Pegaptanib, Nesiritide, Thymalfasin, Defibrotide, Natural alpha interferon OR multiferon, Glatiramer acetate, Preotact, Teicoplanin, Canakinumab, Ipilimumab, Sulodexide, Tocilizumab, Teriparatide, Pertuzumab, Rilonacept, Denosumab, Liraglutide, Golimumab, Belatacept, Buserelin, Velaglucerase alfa, Tesamorelin, Brentuximab vedotin, Taliglucerase alfa, Belimumab, Aflibercept, Asparaginase Erwinia chrysanthemi, Ocriplasmin, Glucarpidase, Teduglutide, Raxibacumab, Certolizumab pegol, Insulin, isophane, Epoetin zeta, Obinutuzumab, Fibrinolysin aka plasmin, Follitropin alpha, Romiplostim, Lucinactant, Natalizumab, Aliskiren, Secukinumab, Somatotropin Recombinant, Drotrecogin alfa, Alefacept, OspA lipoprotein, Urokinase, Abarelix, Sermorelin, Aprotinin, Gemtuzumab ozogamicin, Satumomab Pendetide, Albiglutide, Alirocumab, Ancestim, Antithrombin Alfa, Antithrombin III human, Asfotase Alfa, Atezolizumab, Autologous cultured chondrocytes, Beractant, Blinatumomab, C1 Esterase Inhibitor (Human), Coagulation Factor XIII A-Subunit (Recombinant), Conestat alfa, Daratumumab, Desirudin, Dulaglutide, Elosulfase alfa, Elotuzumab, Evolocumab, Fibrinogen Concentrate (Human), Filgrastim-sndz, Gastric intrinsic factor, Hepatitis B immune globulin, Human calcitonin, Human Clostridium tetani toxoid immune globulin, Human rabies virus immune globulin, Human Rho(D) immune globulin, Hyaluronidase (Human Recombinant), Idarucizumab, Immune Globulin Human, Vedolizumab, Ustekinumab, Turoctocog alfa, Tuberculin Purified Protein Derivative, Simoctocog Alfa, Siltuximab, Sebelipase alfa, Sacrosidase, Ramucirumab, Prothrombin complex concentrate, Poractant alfa, Pembrolizumab, Peginterferon beta-1a, Ofatumumab, Obiltoxaximab, Nivolumab, Necitumumab, Metreleptin, Methoxy polyethylene glycol-epoetin beta, Mepolizumab, Ixekizumab, Insulin Pork, Insulin Degludec, Insulin Beef, Thyroglobulin, Anthrax immune globulin human, Anti-inhibitor coagulant complex, Anti-thymocyte Globulin (Equine), Anti-thymocyte Globulin (Rabbit), Brodalumab, C1 Esterase Inhibitor (Recombinant), Canakinumab, Chorionic Gonadotropin (Human), Chorionic Gonadotropin (Recombinant), Coagulation factor X human, Dinutuximab, Efmoroctocog alfa, Factor IX Complex (Human), Hepatitis A Vaccine, Human Varicella-Zoster Immune Globulin, Ibritumomab tiuxetan, Lenograstim, Pegloticase, Protamine sulfate, Protein S human, Sipuleucel-T fusion antigen prostatic acid phosphatase with granulocyte-macrophage colon stimulating factor, Somatropin recombinant, Susoctocog alfa, Thrombomodulin Alfa, Cas9, and Cpf1 (Cas12a).

The term “catalyst” as used in the present application refers to a substance that starts or speeds up a chemical reaction while undergoing no permanent change itself. Examples include Shvo's catalyst, RuHCl(CO)(PPh₃)₃, RuCl₂(PPh₃)₃, RhH(PPh₃)₄, [(neocuproine)PdOAc]₂OTf₂, [(C₄Ph₄CO)(CO)₂Ru]₂, RuH₂(PPh₃)₄, Ru₃(CO)₁₂, RuH₂(PPh₃)₄, RuH₂(PMe₃)₄, RuH₂(PBu₃)₄, RuH₂(DMPE)₂, (PMe₃)₂Ru(2-methylallyl)₂, (DMPE)Ru(2-methylallyl)₂, (DIOP)Ru(2-methylallyl)₂, Shvo amide analogue as described in (Zhao and Hartwig 2005), trans-RuHCl(tmen)(BINAP), (DPPF)RuCl₂(eda), (PPh₃)₂RuCl₂(eda), (DIOP)RuCl₂(eda), (PMe₃)₂RuCl₂(eda), RuH₂(CO)(PMe₃)₃, cis-(PMe₃)₂RuCl₂(eda), [RuCl₂(η⁶-benzene)]₂, RuH₄(Ph₃P)₃, Cp*RuCl[Ph₂P(CH₂)₂NH₂-κ2-P,N], (1,3-bis(6′-methyl-2′-pyridylimino)isoindoline) pincer Ru(II) hydride complex as described in (Tseng, Kampf, and Szymczak 2013), Ir-containing dibenzobarrelene-based PC(sp₃)P pincer complex as described in (Musa et al. 2011), a sol-gel entrapped iridium-pincer catalyst as described in (Oded et al. 2012), FeOx as described in (Huang et al., 2008), IrH₅(iPr₃P)₂, ReH₇(iPr₃P)₂, RhH(PPh₃)₄-benzalacetone, Supported gold nanoparticles as described in (Mitsudome et al., 2009), Cp*IrCl[OCH₂C(C₆H₅)₂NH₂], RuCl₂(2-aminomethylpyridine)(dppb) as well as it's methylated analogue trans-RuCl₂(dppb)(2-(N,N-dimethylamino)methylpyridine), RuCl₂(2-aminomethylpyridine)(dppf) as well as it's methylated analogue trans-RuCl₂(dppf)(2-(N,N-dimethylamino)methylpyridine), RuCl₂(PPh₃)₂(2-aminomethylpyridine), RuCl₂(N,N′-dimethylethylenediamine)(dppf), RuCl₂(PPh₃)(dppb), Cyclopentadienone triazolylidene ruthenium(0) complexes as described in page 49 of (Cesari 2016), Hydroxycyclopentadienyl and methoxycyclopentadienyl N-heterocyclic carbene ruthenium(II) complexes as described in page 66 of (Cesari 2016), Ruthenium tetraarylcyclopentadienone NHC-decorate dendrimers as described in page 101 of (Cesari 2016), Ruthenium 2,5-bis-tetrimethylsilane-3,4 NHC-decorate dendrimers as described in page 105 of (Cesari 2016), Imidazolium salts of ruthenium anionic cyclopentadienone complexes as described in page 117 of (Cesari 2016), [Ru(OCORF)₂(CO)(PPh₃)₂] where RF=CF3, C2F5, or C6F5, [RuH₂(N₂)(PPh₃)₃], [RuHCl(CO)(HN(C₂H₄PⁱPr₂)₂)], Diruthenium(II,II) complex [Ru,(L1)(OAc)₃]Cl, spanned by a naphthyridine-diimine ligand and bridged by three acetates as described in (Dutta et al. 2016), Ruthenium boryl complexes such as [2,5-Ph₂-3,4-Tol²(η⁵-C₄COBcat)Ru(CO)₂Bcat] and [2,5-Ph2-3,4-Tol₂(η⁵-C₄COH)Ru(CO)₂Bcat, Hydroxycyclopentadienylruthenium hydride dimer (Shvo's type) complexes as described in page 158 of (Cesari 2016), Shvo-type Organoruthenium complexes as described in (Apps et al. 2014), N-heterocyclic carbene based ruthenium complexes as described in (Zhang et al. 2010), Iron cyclopentadienone N-heterocyclic carbene complexes as described in page 133 of (Cesari 2016), [Os(OCORF)₂(CO)(PPh₃)₂] where RF=CF3, C2F5, or C6F5, IrH₅(i-Pr₃P)₂, [Rh(2,2′-bipyridyl)₂]Cl, IrH(Cl)[2,6-(tBu₂PO)₂C₆H₃], {IrH(acetone)[2,6-(tBu₂PO)₂C₆H₃]}{BF4}, IrH(Cl)[{2,5-(tBu₂PCH₂)₂C₅H₂}Ru(C₅H₅)], iridium triazine-pincer complex as described in (Michlik and Kempe 2013), and Cyclopentadienyl Ir(III) complexes bearing non-innocent pyridine ligands as described in (Fujita, Tanino, and Yamaguchi 2007), and [Ph₄(η⁴-C₄CO)]Fe(CO)₃.

The term “Shvo's catalyst” as used in the present application refers to 1-Hydroxytetraphenylcyclopentadienyl-(tetraphenyl-2,4-cyclopentadien-1-one)-μ-hydrotetracarbonyldiruthenium(II) which has the following structure:

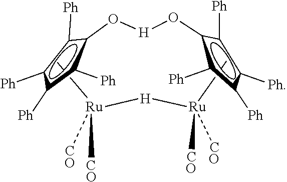

In the present application the expression “site-specifically modified” refers to any method which preferentially modifies the protein at one particular point.

The term “transition metal” as used in the present application refers to an element whose atom has a partially filled d sub-shell, or which can give rise to cations with an incomplete d sub-shell.

The term “d-block element” as used in the present application refers to an element which can be found in group 3 to 12 of the modern periodic table, inclusive.

The terms “treatment” and “therapy,” as used in the present application, refer to a set of hygienic, pharmacological, surgical and/or physical means used with the intent to cure and/or alleviate a disease and/or symptom with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

### Chemical Synthesis

In a first aspect, the present disclosure provides a method for synthesizing a handle-substituted carbohydrate lactone comprising contacting a handle-substituted aldose with a catalyst under aprotic conditions in the presence of a hydrogen acceptor.

In some embodiments, the handle-substituted carbohydrate lactone has a five-, six- or seven-membered ring. In some embodiments, the carbohydrate lactone is a 1,5-lactone. In some embodiments, the aldose is a furanose, pyranose or septanose. In some embodiments, the carbohydrate lactone has a five-, six- or seven-membered ring and C6, C4 and/or C3 is substituted with a handle.

The handle-substituted aldose may be a handle-substituted derivative of D-ribose, D-arabinose, D-xylose, D-lyxose, L-ribose, L-arabinose, L-xylose, or L-lyxose, D-allose, D-altrose, D-glucose, D-mannose, D-gulose, D-idose, D-galactose, D-talose, L-allose, L-altrose, L-glucose, L-mannose, L-gulose, L-idose, L-galactose, or L-Talose. Heptose- and Hexose-derivatives may also be C6-substituted according to the disclosure. Heptose-derivatives may also be C7-handle-substituted according to the disclosure.

In some embodiments, the handle-substituted aldose is a ribose substituted at positions C2, C3, C4 or C5 with an azide (Hobbs and Eckstein 1977; Hanessian et al. 2005; Hassan and Slama 1992; Singh et al. 2008), an arabinose substituted at positions C2, C4 or C5 with an azide (Bols et al. 1988; Müller et al. 2010; Singh et al. 2008), a xylose substituted at position C2, C3, C4 or C5 (Bols et al. 1988; McDevitt and Lansbury 1996; Sinha and Brew 1980), an allose substituted at positions C3, C5, or C6 with an azide (Worch and Wittmann 2008; Hudlicky, Nugent, and Griffith 1994; Roy and Sanjayan 2012), an altose substituted at positions C2, C3, C5 or C6 with an azide (Guthrie and Murphy 1963; Uriel and Santoyo-González 1999; Kefurt et al. 1986), a glucose substituted at positions C2, C3, C4, C5 or C6 with an azide (Zaro et al. 2017; Nagy et al. 2017; Sinha and Brew 1980; Hudlicky, Nugent, and Griffith 1994; Györgydeàk and Szilàgyi 1987), a mannose substituted at positions C2, C3, C4 or C5 with an azide (Augé, David, and Malleron 1989; Fitz, Schwark, and Wong 1995; Khedri et al. 2014; Dharuman, Wang, and Crich 2016; Ligeour et al. 2015), a gulose substituted at positions C3 or C5 with an azide (Campo et al. 2012; Hudlicky, Nugent, and Griffith 1994), an idose substituted at positions C5 or C6 with an azide (Berger et al. 1992; Hamagami et al. 2016), a galactose substituted at positions C2, C3, C4, C5 or C6 with an azide (Ahad, Jensen, and Jewett 2013; Lowary and Hindsgaul 1994; Zou et al. 2012; Uriel and Santoyo-González 1999; Zou et al. 2012) or a talose substituted at positions C5 or C6 with an azide (Ayers et al. 2012; Kefurt, Kefurtová, and Jarý 1988).

In some embodiments, the handle-substituted aldose is 2-azido-2-deoxy-D-glucose (CAS 56883-39-7, Cat. AG753, Synthose), 2-azido-2-deoxy-D-glucose (CAS 56883-39-7, Cat. MA02624, Carbosynth), 2-azido-2-deoxy-D-galactose (CAS 68733-26-6, Cat. MA03562, Carbosynth), 2-azido-2-deoxy-D-galactose (CAS 68733-26-6, Cat. AL229, Synthose), 3-azido-3-deoxy-D-glucopyranose (CAS 104875-44-7, Cat. AG915, Synthose), 3-azido-3-deoxy-D-galactose (CAS 2250404-17-0, Cat. AL491, Synthose), 4-azido-4-deoxy-D-glucopyranose (CAS 74593-35-4, Cat. AG397, Synthose), 4-azido-4-deoxy-D-glucose (CAS 74593-35-4, Cat. MA106335, Carbosynth), 4-azido-4-deoxy-D-galactose (CAS 94885-19-5, Cat. AL788, Synthose), 6-azido-6-deoxy-D-glucopyranose (CAS 2089473-19-6, Cat. AG413, Synthose), 6-azido-6-deoxy-D-glucose (CAS 20847-05-6, Cat. MA02620, Carbosynth), 6-azido-6-deoxy-D-mannose (CAS 316379-15-4, Cat. MA167258, Carbosynth), 6-azido-6-deoxy-D-galactose (CAS, 66927-03-5, Cat. MA02618, Carbosynth), 6-azido-6-deoxy-D-galactose (CAS 66927-03-5, Cat. AF432, Synthose), 6-azido-6-deoxy-L-galactose (alternative name: 6-azido-L-fucose, CAS 70932-63-7, Cat. AF415, Synthose) or 6-azido-6-deoxy-L-galactose (CAS 70932-63-7, Cat. MA08355, Carbosynth).

In some embodiments, the handle-substituted aldose is a handle-substituted ribose, arabinose, xylose, allose, glucose, mannose or galactose.

In some embodiments, the aldose and carbohydrate lactone is substituted with any one of the following handles: Azide, Isothiocyanate, Isocyanate, Acyl azide, NHS ester, in situ NHS ester, Hydroxybenzotriazole (HOBt) ester, Sulfonyl chloride, -flouride, Sulfonyl ester (e.g., tosyl, mesyl, trifyl, tresyl esters etc.), Aldehyde, ketone, dicarbonyl, Aldehyde, Amine, Epoxide, Carbonate (e.g., succinimidyl carbonate), Cyclic imidocarbonate, NHS carbonate, N,N′-disuccinimidyl carbonate (DSC), Cyanate ester, Carbamate (e.g., imidazole carbamate), Acyl imidazole, NHS carbamate, Aryl halide (e.g., fluorobenzene derivatives), Haloacetyl or alkyl halide, Imidoester (imidates), Carboxylate, Acid anhydride, Fluorophenyl ester (pentafluorophenyl/PFP, tetrafluorophenyl/TFP, sulfo-tetrafluorophenyl/STP esters), Tris(hydroxymethyl)phosphine/THP, beta-[tris(hydroxymethyl)phospino]proprionic acid/THPP, Cyclic hemiacetal, Azlactone, Activated double bond, Methylpyridinium ether, 6-sulfo-cytosine derivative, activated halogen, haloacetyl derivative (e.g., iodoacetyl), benzyl halide, and alkyl halide (N- and S-mustards) derivative, Activated double bond, Vinyl sulfone, Maleimide, Aziridine, Aryl halide, Acryloyl derivative (e.g., acrylic and methacrylic acid derivatives), Disulfide (e.g., protected thiol, such as, pyridyl disulfide deriv., 5-thiol-2-nitrobenzoic acid, TNB-thiol—obtainable by treating a sulfhyrdyl with Ellman's reagent), Thiol (free), Thioester (e.g., phenylthioester), Cysteine attached via C-terminus, Cisplatin derivative, 2-cyanobenzothiazole, Diazoalkane compound, diazoacetyl compound, Epoxide, Aryl azide (e.g., phenyl azide, perfluorinated phenyl azide), Benzophenone, Anthraquinone, Diazo compound (e.g., diazotrifluoropropionate, diazopyruvate), Diazirene derivative, Psoralen compound, Halogenated phenyl azide, Alkene, Diene, Furan, Nitrone, Tetrazine, nitrile oxide, diazoalkane, syndone, quadricyclane, Boronic acid (e.g., phenyl boronic acid, and phenyldiboronic acid), BCN, BCN, 1,3-Dithiolium-4-olate, phosphine (e.g., triphenyl phosphine with electrophilic trap (Staudinger ligation)), phosphine derivative (e.g., bi- or tri-phenyl aryl ester, or acyl imidazole for traceless Staudinger ligation)), alkene, alkyne, strained alkyne, OCT, MOFO, DIFO, DIFO2, DIFO3, DIMAC, DIBO, DIBAC (ADIBO), BARAC, BCN, Sondheimer diyene, TMDIBO, S-DIBO, COMBO, PYRROC, TMTH, DIFBO, ALO, isonitrile, thioalkyne, keto-DIBO, strained olefin, trifluoromethyl-substituted oxanorbornadiene, Biotin, biotin derivative, boronic acid, p-boronophenylalanine, strained dialkyne or Sondheimer dialkyne. In a preferred embodiment, the aldose and carbohydrate lactone is substituted with any one of the following handles: azide, Cyclic hemiacetal, Aziridine, Cysteine attached via C-terminus, Aminooxy groups (also known as alkoxyamines), Hydrazine (Hydrazide), Diazo compound (i.e., diazotrifluoropropionate, diazopyruvate), diazoalkane, isonitrile (aka isocyanide, carbylamine), boronic acid, p-boronophenylalanine, Amine, Aldehyde, ketone, dicarbonyl (i.e., phenylglyoxal deriv.), Aldehyde, Vinyl sulfone, Maleimide, Acryloyl derivative (e.g., acrylic and methacrylic acid derivative), Disulfide (e.g., protected thiol, such as, pyridyl disulfide deriv., 5-thiol-2-nitrobenzoic acid, TNB-thiol), Thioester (e.g., phenylthioester (on C-terminal peptides for native chemical ligation)), 2-cyanobenzothiazole, Alkene, Diene, Furan, Nitrone, Tetrazine, Tetrazine, nitrile oxides, syndone, quadricyclane, BCN, BCN, 1,3-Dithiolium-4-olate, phosphine (e.g., triphenyl phosphine with electrophilic trap), phosphine derivative (bi- or tri-phenyl aryl ester, acyl imidazole for traceless Staudinger ligation), alkene, alkyne, strained alkyne, OCT, MOFO, DIFO, DIFO2, DIFO3, DIMAC, DIBO, DIBAC (ADIBO), BARAC, BCN, Sondheimer diyene, TMDIBO, S-DIBO, COMBO, PYRROC, TMTH, DIFBO, ALO, thioalkyne, keto-DIBO, strained olefin, trifluoromethyl-substituted oxanorbornadiene, Biotin or biotin derivative. In a more preferred embodiment, the aldose and carbohydrate lactone is substituted with any one of the following handles: azide, Aldehyde, ketone, dicarbonyl (e.g., phenylglyoxal deriv.), Vinyl sulfone, Maleimide, Acryloyl derivative (e.g., acrylic and methacrylic acid derivative), Disulfide (e.g., protected thiol, such as, pyridyl disulfide deriv., 5-thiol-2-nitrobenzoic acid, TNB-thiol), thioester (e.g., phenylthioester), 2-cyanobenzothiazole, Alkene, Diene, Furan, Nitrone, Tetrazine, nitrile oxide, syndone, quadricyclane, BCN, BCN, 1,3-Dithiolium-4-olate, phosphine (e.g., triphenyl phosphine with electrophilic trap), phosphine derivative (bi- or tri-phenyl aryl ester, acyl imidazole), alkene, alkyne, strained alkynes, OCT, MOFO, DIFO, DIFO2, DIFO3, DIMAC, DIBO, DIBAC, BARAC, BCN, Sondheimer diyene, TMDIBO, S-DIBO, COMBO, PYRROC, TMTH, DIFBO, ALO, thioalkyne, keto-DIBO, strained olefin, trifluoromethyl-substituted oxanorbornadiene, Biotin or biotin derivative. Preferably, the handle is an azide.

In some embodiments the handle comprises a linker through which it is attached to the carbohydrate lactone.

In some embodiments, the handle-substituted aldose is 6-azido-6-deoxy-D-glucose, 4-azido-4-deoxy-D-glucose or 3-azido-3-deoxy-D-glucose.

The catalyst may be a transition metal-based catalyst. Preferably a ruthenium- or palladium-based catalyst.

In some embodiments, the catalyst is Shvo's catalyst, RuHCl(CO)(PPh₃)₃, RuCl₂(PPh₃)₃, RhH(PPh₃)₄, [(neocuproine)PdOAc]₂OTf₂, [(C₄Ph₄CO)(CO)₂Ru]₂, RuH₂(PPh₃)₄, Ru₃(CO)₁₂, RuH₂(PPh₃)₄, RuH₂(PMe₃)₄, RuH₂(PBu₃)₄, RuH₂(DMPE)₂, (PMe₃)₂Ru(2-methylallyl)₂, (DMPE)Ru(2-methylallyl)₂, (DIOP)Ru(2-methylallyl)₂, Shvo amide analogue as described in (Zhao and Hartwig 2005), trans-RuHCl(tmen)(BINAP), (DPPF)RuCl₂(eda), (PPh₃)₂RuCl₂(eda), (DIOP)RuCl₂(eda), (PMe₃)₂RuCl₂(eda), RuH₂(CO)(PMe₃)₃, cis-(PMe₃)₂RuCl₂(eda), [RuCl₂(η⁶-benzene)]₂, RuH₄(Ph₃P)₃, Cp*RuCl[Ph₂P(CH₂)₂NH₂-κ2-P,N], (1,3-bis(6′-methyl-2′-pyridylimino)isoindoline) pincer Ru(II) hydride complex as described in (Tseng, Kampf, and Szymczak 2013), Ir-containing dibenzobarrelene-based PC(sp₃)P pincer complex as described in (Musa et al. 2011), a sol-gel entrapped iridium-pincer catalyst as described in (Oded et al. 2012), FeOx as described in (Huang et al., 2008), IrH₅(iPr₃P)₂, ReH₇(iPr₃P)₂, RhH(PPh₃)₄-benzalacetone, Supported gold nanoparticles as described in (Mitsudome et al., 2009), Cp*IrCl[OCH₂C(C₆H₅)₂NH₂], RuCl₂(2-aminomethylpyridine)(dppb) as well as it's methylated analogue trans-RuCl₂(dppb)(2-(N,N-dimethylamino)methylpyridine), RuCl₂(2-aminomethylpyridine)(dppf) as well as it's methylated analogue trans-RuCl₂(dppf)(2-(N,N-dimethylamino)methylpyridine), RuCl₂(PPh₃)₂(2-aminomethylpyridine), RuCl₂(N,N′-dimethylethylenediamine)(dppf), RuCl₂(PPh₃)(dppb), Cyclopentadienone triazolylidene ruthenium(0) complexes as described in page 49 of (Cesari 2016), Hydroxycyclopentadienyl and methoxycyclopentadienyl N-heterocyclic carbene ruthenium(II) complexes as described in page 66 of (Cesari 2016), Ruthenium tetraarylcyclopentadienone NHC-decorate dendrimers as described in page 101 of (Cesari 2016), Ruthenium 2,5-bis-tetrimethylsilane-3,4 NHC-decorate dendrimers as described in page 105 of (Cesari 2016), Imidazolium salts of ruthenium anionic cyclopentadienone complexes as described in page 117 of (Cesari 2016), [Ru(OCORF)₂(CO)(PPh₃)₂] where RF=CF3, C2F5, or C6F5, [RuH₂(N₂)(PPh₃)₃], [RuHCl(CO)(HN(C₂H₄PⁱPr₂)₂)], Diruthenium(II,II) complex [Ru,(L1)(OAc)₃]Cl, spanned by a naphthyridine-diimine ligand and bridged by three acetates as described in (Dutta et al. 2016), Ruthenium boryl complexes such as [2,5-Ph₂-3,4-Tol²(η⁵-C₄COBcat)Ru(CO)₂Bcat] and [2,5-Ph2-3,4-Tol₂(η⁵-C₄COH)Ru(CO)₂Bcat, Hydroxycyclopentadienylruthenium hydride dimer (Shvo's type) complexes as described in page 158 of (Cesari 2016), Shvo-type Organoruthenium complexes as described in (Apps et al. 2014), N-heterocyclic carbene based ruthenium complexes as described in (Zhang et al. 2010), Iron cyclopentadienone N-heterocyclic carbene complexes as described in page 133 of (Cesari 2016), [Os(OCORF)₂(CO)(PPh₃)₂] where RF=CF3, C2F5, or C6F5, IrH₅(i-Pr₃P)₂, [Rh(2,2′-bipyridyl)₂]Cl, IrH(Cl)[2,6-(tBu₂PO)₂C₆H₃], {IrH(acetone)[2,6-(tBu₂PO)₂C₆H₃]}{BF4}, IrH(Cl)[{2,5-(tBu₂PCH₂)₂C₅H₂}Ru(C₅H₅)], iridium triazine-pincer complex as described in (Michlik and Kempe 2013), and Cyclopentadienyl Ir(III) complexes bearing non-innocent pyridine ligands as described in (Fujita, Tanino, and Yamaguchi 2007), or [Ph₄(η⁴-C₄CO)]Fe(CO)₃. Preferably, the catalyst is Shvo's catalyst, RuHCl(CO)(PPh₃)₃, RuCl₂(PPh₃)₃, RhH(PPh₃)₄, [(neocuproine)PdOAc]₂OTf₂, [(C₄Ph₄CO)(CO)₂Ru]₂, RuH₂(PPh₃)₄, Ru₃(CO)₁₂, RuH₂(PPh₃)₄, RuH₂(PMe₃)₄, RuH₂(PBu₃)₄, RuH₂(DMPE)₂, (PMe₃)₂Ru(2-methylallyl)₂, (DMPE)Ru(2-methylallyl)₂, (DIOP)Ru(2-methylallyl)₂, Shvo amide analogue as described in (Zhao and Hartwig 2005), trans-RuHCl(tmen)(BINAP), (DPPF)RuCl₂(eda), (PPh₃)₂RuCl₂(eda), (DIOP)RuCl₂(eda), (PMe₃)₂RuCl₂(eda), RuH₂(CO)(PMe₃)₃, cis-(PMe₃)₂RuCl₂(eda), [RuCl₂(η⁶-benzene)]₂, RuH₄(Ph₃P)₃, Cp*RuCl[Ph₂P(CH₂)₂NH₂-κ2-P,N], (1,3-bis(6′-methyl-2′-pyridylimino)isoindoline) pincer Ru(II) hydride complex as described in (Tseng, Kampf, and Szymczak 2013), Ir-containing dibenzobarrelene-based PC(sp₃)P pincer complex as described in (Musa et al. 2011), and a sol-gel entrapped iridium-pincer catalyst as described in (Oded et al. 2012), FeOx as described in (Huang et al., 2008), IrH₅(iPr₃P)₂, ReH₇(iPr₃P)₂, RhH(PPh₃)₄-benzalacetone and Supported gold nanoparticles as described in (Mitsudome et al., 2009) or Cp*IrCl[OCH₂C(C₆H₅)₂NH₂]. More preferably, the catalyst is Shvo's catalyst, RuHCl(CO)(PPh₃)₃, RuCl₂(PPh₃)₃, RhH(PPh₃)₄, or [(neocuproine)PdOAc]₂OTf₂.

In some embodiments, the present disclosure provides a method for synthesizing an handle-substituted six-membered 1,5-carbohydrate-lactone comprising contacting an handle-substituted pyranose with a catalyst under aprotic conditions in the presence of a hydrogen acceptor.

In some embodiments, the present disclosure provides a method for synthesizing an azido-substituted six-membered 1,5-carbohydrate-lactone comprising contacting an azido-substituted pyranose with a catalyst under aprotic conditions in the presence of a hydrogen acceptor.

In some embodiments, the present disclosure provides a method for synthesizing an azido-substituted six-membered 1,5-carbohydrate-lactone comprising contacting an azido-substituted pyranose with a ruthenium- or palladium-based catalyst under aprotic conditions in the presence of a hydrogen acceptor.

The ruthenium- or palladium-based catalyst may be any ruthenium- or palladium-based catalyst known in the art such as hydridochlorocarbonyltris(triphenyl-phosphine)ruthenium(II), dihydridotetrakis(triphenylphosphine)ruthenium(II), [(neocuproine)PdOAc]₂OTf₂ and Shvo's catalyst. In some embodiments, the catalyst is immobilized onto a solid phase surface (see He and Horváth, 2017). In some embodiments, the catalyst is Shvo's catalyst.

In some embodiments, the hydrogen acceptor is a compound comprising a ketone, carbonyl, alkene, alkyne and/or imine group. In some embodiments, the hydrogen acceptor is also an aprotic solvent. In some embodiments, the hydrogen acceptor is cyclohexanone, cycloheptanone, benzalacetone, acetone, 3-pentanone, acetophenone, 4-nitro-acetophenone, 4-fluoro-acetophenone, 4-methoxy-acetophenone, butanone (methyl ethyl ketone), but-3-en-2-one, 3-methylbutan-2-one, 2-pentanone, 1-penten-3-one, 3,3-dimethyl-2-butanone, 2-methylpentan-3-one, 3-methyl-2-pentanone, 4-methylpentan-2-one (methyl isobutyl ketone), 3-penten-2-one, 3-hexanone (acetylacetone), 2-hexanone, 5-hexen-2-one, 4-methyl-3-penten-2-one, dibenzylideneacetone, cyclopentanone, 3-methyl-3-penten-2-one, 5-methylhexan-3-one, 4-methyl-2-hexanone, 2-heptanone (methyl n-amyl ketone), 4-heptanone, 4-octanone, diisobutyl ketone, diacetone alcohol (4-hydroxy-4-methylpentan-2-one), 3-octanone, 5-methylhexan-3-one, 2-octanone, oct-1-en-3-one, 5-methyl-2-octanone, ethyl acetoacetate (ethyl 3-oxobutanoate), 2-nonanone, 3-nonanone, 4-nonanone, 5-nonanone, 1-methylpyrrolidin-2-one, isophorone (3,5,5-trimethylcyclohex-2-en-1-one), benzylideneacetone, 2-decanone, 5-decanone, 3-phenyl-5-hexen-2-one, pentane-2,4-dione (acetylacetone), butanedione (butane-2,3-dione), benzil (1,2-diphenylethane-1,2-dione), diphenylacetylene, 1-hexyne, cyclohexene, pentanal, 1-methylcyclohexene, 1-octene, 2,4-dimethyl-3-pentanone, 4-octyne, styrene, diphenylacetylene, benzaldehyde, trans-stilbene, benzophenone, 1,3-diphenyl-2-propanone, anthracene, or 2-pentene. In some embodiments, the hydrogen acceptor is cyclohexanone.

Without being bound by any particular theory, it was hypothesized by the inventors that the most probable reason for the lack of protein reactivity with the substance(s) obtained by bromine oxidation may be the rapid interconversion of 6-azido-6-deoxy-D-glucono-1,5-lactone, if formed in the first place, to the 1,4-lactone and/or the free acid as the major species in aqueous conditions (Isbell and Frush 1933; Bierenstiel and Schlaf 2004). Thus, the oxidation reaction should be performed under aprotic conditions, e.g., using aprotic solvents and hydrogen acceptors. In some embodiments, the aprotic conditions are achieved by using an aprotic solvent. In some embodiments, the aprotic solvent is cyclohexanone which may also function as the hydrogen acceptor. In some embodiments, a mixed solvent system comprising an aprotic solvent that cannot function as a hydrogen acceptor (i.e., a co-solvent) as well as a hydrogen acceptor may be used to achieve the aprotic conditions. The co-solvent may be 1,1,2-trichlorotrifluoroethane, cyclopentane, heptane, hexane, iso-octane, petroleum ether, cyclohexane, n-butyl chloride, toluene, methyl t-butyl ether, o-xylene, chlorobenzene, o-dichlorobenzene, ethyl ether (diethyl ether), dichloromethane, ethylene dichloride, n-butyl acetate, tetrahydrofuran, chloroform, ethyl acetate, methyl acetate, 1,4-dioxane, pyridine, acetonitrile, propylene carbonate, dimethylformamide, dimethylsulfoxide (DMSO), dimethyl acetamide, and 1-methylpyrrolidin-2-one. For example, the mixed solvent system may comprise hexane and cyclohexanone wherein the cyclohexanone is the hydrogen acceptor.

In the examples of the present disclosure, cyclohexanone is reduced to small amounts of cyclohexanol (a protic substance) during synthesis. Thus, some protic impurities or protic reaction products are tolerable and therefore encompassed.

In some embodiments, the handle-substituted pyranose is according to formula (I), (II) or (III):

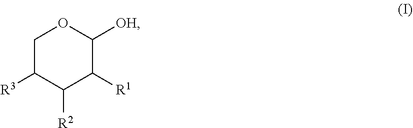

wherein:
(i) R¹, R² and R³ are each independently hydrogen, a hydroxyl, a methyl, or a handle,
(ii) at least one of R¹, R² and R³ is a handle, and
(iii) one or none of R¹, R² and R³ is a methyl;

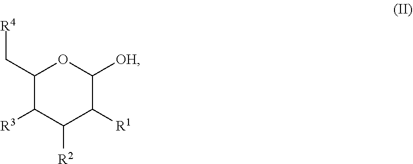

wherein:
(i) R¹, R² and R³ are each independently hydrogen, a hydroxyl, a methyl, or a handle,
(ii) R⁴ is hydrogen, a hydroxyl, a methyl, a handle, a carboxyl, an ester or an amide,
(iii) at least one of R¹, R², R³ and R⁴ is a handle, and
(iv) one or none of R¹, R², R³ and R⁴ is a methyl;

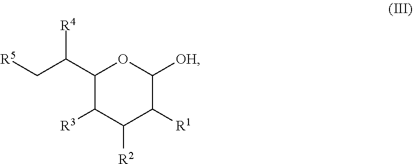

wherein:
(i) R¹, R² and R³ are each independently hydrogen, a hydroxyl, a methyl, or a handle,
(ii) R⁴ and R⁵ are each independently hydrogen, a hydroxyl, a methyl, a handle, or a carboxyl,
(iii) at least one of R¹, R², R³, R⁴ and R⁵ is a handle, and
(iv) one or none of R¹, R², R³, R⁴ and R⁵ is a methyl.

In some embodiments, the azido-substituted pyranose is according to formula (I), (II) or (III):

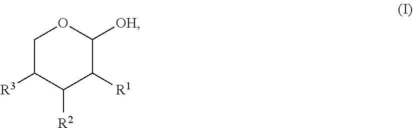

wherein:
(i) R¹, R² and R³ are each independently hydrogen, a hydroxyl, a methyl, or an azide,
(ii) at least one of R¹, R² and R³ is an azide, and
(iii) one or none of R¹, R² and R³ is a methyl;

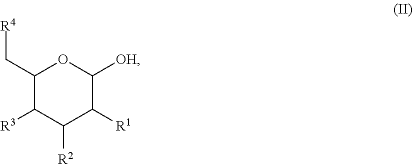

wherein:
(i) R¹, R² and R³ are each independently hydrogen, a hydroxyl, a methyl, or an azide,
(ii) R⁴ is hydrogen, a hydroxyl, a methyl, an azide, a carboxyl, an ester or an amide,
(iii) at least one of R¹, R², R³ and R⁴ is an azide, and
(iv) one or none of R¹, R², R³ and R⁴ is a methyl;

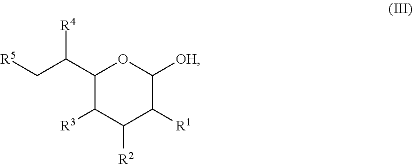

wherein:
(i) R¹, R² and R³ are each independently hydrogen, a hydroxyl, a methyl, or an azide,
(ii) R⁴ and R⁵ are each independently hydrogen, a hydroxyl, a methyl, an azide, or a carboxyl,
(iii) at least one of R¹, R², R³, R⁴ and R⁵ is an azide, and
(iv) one or none of R¹, R², R³, R⁴ and R⁵ is a methyl.

It is further understood that -deoxy, -amine, -amide, -sulfo, -phospho, -ester and -carboxy azido-derivatives of the above monosaccharides may be used in the present disclosure. Disaccharides may also be used. In such embodiments, disaccharides comprise any one of the azido-substituted pyranoses described herein.

In some embodiments, the present disclosure provides a method for synthesizing an azido-substituted six-membered 1,5-carbohydrate-lactone according to formula (V):

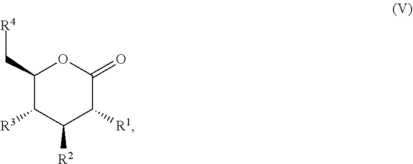

comprising contacting an azido-substituted pyranose with a catalyst under aprotic conditions in the presence of a hydrogen acceptor, wherein the azido-substituted pyranose is according to formula (VI):

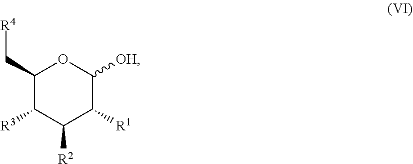

and:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide, and
(iv) one or none of R¹, R², R³ and R⁴ is a methyl.

In some embodiments, the handle-substituted aldose is 6-azido-6-deoxy-D-glucose, 4-azido-4-deoxy-D-glucose or 3-azido-3-deoxy-D-glucose. It is clear to the skilled person that when the handle-substituted aldose is 6-azido-6-deoxy-D-glucose, the resultant handle-substituted carbohydrate lactone obtained by the method of the present disclosure is 6-azido-6-deoxy-D-glucono-1,5-lactone. Similarly, the resultant product obtained when the handle-substituted aldose is 4-azido-4-deoxy-D-glucose is 4-azido-4-deoxy-D-glucono-1,5-lactone, and when the handle-substituted aldose is 3-azido-3-deoxy-D-glucose the resultant product is 3-azido-3-deoxy-D-glucono-1,5-lactone. This reasoning may apply to any handle-substituted aldose and the resultant handle-substituted carbohydrate lactone obtained by the methods of the present disclosure.

In one example, the compound of interest, 6-azido-6-deoxy-D-glucono-1,5-lactone, is purified from the reaction mixture by precipitation with hexanes, followed by resolubilization in a mixture of 5:1 ethyl acetate/acetone (v/v).

It is also conceivable that precipitation of the target compound may be achieved by adding other solvents, which are more hydrophobic than the reaction solvent, causing precipitation of the target compound from the reaction mixture, whilst ideally leaving the catalyst species and possible other impurities in solution.

Thus, in some embodiments the method further comprises precipitating the handle-substituted carbohydrate lactone with an aprotic solvent. The aprotic solvent may be Di-n-butyl phthalate, 1,1-dichloroethane, 3-pentanone, chloroform, methyl acetate, diglyme, dimethoxyethane (glyme), ethyl benzoate, ethyl acetate, tetrahydrofuran (THF), anisole, chlorobenzene, dioxane, diethylamine, methyl t-butyl ether (MTBE), ether, benzene, toluene, p-xylene, carbon disulphide, carbon tetrachloride, heptane, hexane, pentane, cyclohexane, cyclohexanone, 1,1,2-Trichlorotrifluoroethane, Cyclopentane, Petroleum Ether, 2,2,4-Trimethylpentane (isooctane), 1-Chlorobutane, o-Dichlorobenzene, Ethyl Ether (Diethyl ether), Dichloromethane, 1,2-Dichloroethane, n-Butyl Acetate, Methyl Isoamyl Ketone, Tetrahydrofuran, Methyl Isobutyl Ketone, Acetophenone, Methyl n-Propyl Ketone, Cyclohexanone, Tetrachloroethylene, 1,2,4-Trichlorobenzene, 2-Hexanone, 4-Octanone, diisobutyl ketone, 3-Octanone, 5-Methylhexan-3-one, 2-Octanone, Oct-1-en-3-one, 5-Methyl-2-octanone, Cycloheptanone, ethyl acetoacetate (ethyl 3-oxobutanoate), 2-nonanone, 3-Nonanone, 4-Nonanone, 5-Nonanone, 1-Methylpyrrolidin-2-one, Acetophenone, isophorone (3,5,5-trimethylcyclohex-2-en-1-one), Benzylideneacetone, 2-Decanone, or 5-Decanone, or 3-phenyl-5-hexen-2-one.

Amine-containing solvents are generally less preferable, as the amine group may react with carbohydrate-derived lactones. However carefully choosing the solvent and contacting the reaction mixture with such solvents at a reduced temperature and only for a limited time may allow to selectively precipitate the target compound without causing too much loss. Examples of such solvents are: N,N-dimethylaniline, and diethylamine.

After precipitation, the handle-substituted carbohydrate lactone may be re-solubilized. To achieve higher purity after the initial precipitation step, resolubilization may be achieved with a solvent that preferably selectively dissolves the target compound (i.e., the handle-substituted carbohydrate lactone) but not the catalyst nor other impurities. A combination of solvents may also be used to solubilize the target compound.

The solid remainders, e.g., the catalyst and/or other impurities may be removed by centrifugation, decanting, filtering, and/or other solid-liquid separation methods or a combination thereof.

Aprotic solvents are preferred solvents for the solubilization of the target compound. Protic solvents are less preferable for the solubilization of the target compound, because they may open up the lactone ring to the linear chain (acid), or they may cause the target to re-arrange from, e.g., the 1,5- to the 1,4-lactone.

In some embodiments, the method further comprises a resolubilization step wherein the precipitated handle-substituted carbohydrate lactone is redissolved in a composition comprising Cyclohexanone, 2-pentanone, 2-butanone, benzonitrile, acetone, dimethylformamide (DMF), dimethylsulfoxide (DMSO), acetonitrile, acetyl acetone, ethyl acetoacetate, diethylene glycol, ethylene glycol, Methyl Ethyl Ketone, 1,4-Dioxane, Pyridine, Propylene Carbonate, N,N-Dimethylformamide, Dimethyl Acetamide, N-Methylpyrrolidone, dimethylsulfoxide, Tetrachloroethylene, 1,2,4-Trichlorobenzene, Butanone (methyl ethyl ketone), But-3-en-2-one, 3-Methylbutan-2-one, 3-Pentanone, 1-Penten-3-one, 3,3-Dimethyl-2-butanone, 2-Methylpentan-3-one, 3-Methyl-2-pentanone, 4-Methylpentan-2-one (methyl isobutyl ketone), 3-Penten-2-one, 3-Hexanone (acetylacetone), 2-Hexanone, 5-Hexen-2-one, 4-Methyl-3-penten-2-one, Dibenzylideneacetone, Cyclopentanone, t-butyl alcohol, 3-pentanol, 2-pentanol, 2-butanol, cyclohexanol, 1-octanol, 2-propanol, 1-heptanol, i-butanol, 1-hexanol, 1-pentanol, 1-butanol, benzyl alcohol, 1-propanol, acetic acid, ethanol, methanol, glycerine, heavy water, water, Pyridine, 2-aminoethanol, aniline, N,N-dimethylaniline, or diethylamine. Preferably, redissolved in a composition comprising Cyclohexanone, 2-pentanone, 2-butanone, benzonitrile, acetone, dimethylformamide (DMF), dimethylsulfoxide (DMSO), acetonitrile, acetyl acetone, ethyl acetoacetate, diethylene glycol, ethylene glycol, Methyl Ethyl Ketone, 1,4-Dioxane, Pyridine, Propylene Carbonate, N,N-Dimethylformamide, Dimethyl Acetamide, N-Methylpyrrolidone, dimethylsulfoxide, Tetrachloroethylene, 1,2,4-Trichlorobenzene, Butanone (methyl ethyl ketone), But-3-en-2-one, 3-Methylbutan-2-one, 3-Pentanone, 1-Penten-3-one, 3,3-Dimethyl-2-butanone, 2-Methylpentan-3-one, 3-Methyl-2-pentanone, 4-Methylpentan-2-one (methyl isobutyl ketone), 3-Penten-2-one, 3-Hexanone (acetylacetone), 2-Hexanone, 5-Hexen-2-one, 4-Methyl-3-penten-2-one, Dibenzylideneacetone, or Cyclopentanone. More preferably, redissolved in a composition comprising acetone, acetonitrile, ethyl acetate or mixtures thereof. Most preferably, redissolved in a composition comprising ethyl acetate and acetone.

In some embodiments, the method further comprises precipitating the azido-substituted six-membered 1,5-carbohydrate-lactone using hexane followed by re-solubilizing the precipitate in a mixture of ethyl acetate/acetone. In some embodiments, the mixture of ethyl acetate/acetone has a 5:1 ratio of ethyl acetate:acetone.

In some embodiments, the present disclosure provides a method for synthesizing 6-azido-6-deoxy-D-glucono-1,5-lactone comprising contacting 6-azido-6-deoxy-D-glucose with a catalyst under aprotic conditions in the presence of a hydrogen acceptor. In some embodiments, this method further comprises precipitating the azido-substituted 6-azido-6-deoxy-D-glucono-1,5-lactone using hexane followed by re-solubilizing the precipitate in a mixture of ethyl acetate/acetone. In some embodiments, the mixture of ethyl acetate/acetone has a 5:1 ratio of ethyl acetate:acetone.

In some embodiments, the present disclosure provides a method for synthesizing 6-azido-6-deoxy-D-glucono-1,5-lactone comprising contacting 6-azido-6-deoxy-D-glucose with a catalyst under aprotic conditions in the presence of a hydrogen acceptor. In some embodiments, this method further comprises precipitating the azido-substituted 6-azido-6-deoxy-D-glucono-1,5-lactone using hexane followed by re-solubilizing the precipitate in acetonitrile.

In some embodiments, the present disclosure provides a method for synthesizing 6-azido-6-deoxy-D-glucono-1,5-lactone comprising contacting 6-azido-6-deoxy-D-glucose with Shvo's catalyst under aprotic conditions in the presence of a hydrogen acceptor. In some embodiments, this method further comprises precipitating the 6-azido-6-deoxy-D-glucono-1,5-lactone using hexane followed by re-solubilizing the precipitate in a mixture of ethyl acetate/acetone. In some embodiments, the mixture of ethyl acetate/acetone has a 5:1 ratio of ethyl acetate:acetone.

Alternative purification strategies are also envisioned by the present application. It is also conceivable that the lactonization reaction may be carried out in conditions in which the catalyst is fully dissolved in an aprotic solvent(s), and the product is in equilibrium between solid and dissolved states (Bierenstiel 2005, 52). In this case simple solid-liquid separation methods such as decantation, filtration, centrifugation can be performed to separate the desired solid product. To achieve higher purity, optional washing of the solid compound of interest with the suitable solvents can be performed, which ideally removes impurities, whilst leaving the solid product of interest behind. Alternatively, to remove potential traces of catalyst, the resolubilization of compound is affected as before, followed by decantation, filtration, centrifugation, or any other solid-liquid separation method in order to leave the solid impurities behind.

It is also conceivable that the reaction mixture is first evaporated, and the catalyst and other impurities are then removed by washing the residue with a solvent or a solvent mixture(s), which solubilizes the impurities but leaves the solid target compound insoluble. Decantation, filtration, centrifugation, or any other solid-liquid separation methods may be employed to collect the solid.

It is further conceivable that that the reaction mixture is first evaporated, and the target compound is selectively solubilized with a solvent or a solvent mixture as before, whilst leaving impurities behind as a solid.

It is also conceivable that catalyst is removed by binding it to a solid support after the reaction, such as a metal-binding solid support followed by magnetic field separation (for magnetic field-responsive solid support), filtration, decantation, centrifugation, or any other solid-liquid separation techniques.

It is also conceivable that when catalyst is already immobilized during the reaction with the substrate, that the catalyst is removed by, e.g., magnetic field separation (for magnetic field-responsive solid support), filtration, decantation, centrifugation, or any other solid-liquid separation techniques.

It is also conceivable that separation of desired compound and catalyst is achieved by chemically modifying catalyst after the reaction. This may be achieved by techniques such as ligand displacement reactions and/or by “deactivation”; in principle changing the hydrophilicity/hydrophobicity of the catalyst and thus making it easier to purify the compound of interest.

The present disclosure also provides a composition comprising the handle-substituted carbohydrate lactone obtained or obtainable by any one of the methods of the present disclosure. In some embodiments, the purity of the handle-substituted carbohydrate lactone is at least 85%, optionally, as determined by 1D ¹H-, 1D ¹³C-, and/or 2D ¹H-¹³C HSQC NMR. In some embodiments, the purity of the handle-substituted carbohydrate lactone is at least 95%, optionally, as determined by 1D ¹H-, 1D ¹³C-, and/or 2D ¹H-¹³C HSQC NMR. The solvent used to determine the purity for all embodiments may be DMSO-d6.

It is also conceivable that crystallization, also optional recrystallization, may be performed to obtain even purer compound of interest. In this case, the compound is dissolved into a suitable solvent and concentrated by evaporation under normal or reduced pressure. Seeding with seed crystals may help grow purer crystals and mother liquors containing impurities are removed. It is conceivable to use other suitable general methods for crystallization and/or re-crystallization, which are known in the art, such as but not limited to, Single-solvent (re)-crystallization, Multi-solvent (re)-crystallization, cooling (re)-crystallization, evaporative (re)-crystallization, draft tube and baffle (re)-crystallization, hot filtration-(re)-crystallization, seeding, Slow diffusion (re)-crystallization, or Interface/slow mixing (re)-crystallization.

In a further aspect, the present disclosure provides a crystalline form of the handle-substituted carbohydrate lactone obtained or obtainable by any one of the methods of the present disclosure. In some embodiments, the handle-substituted carbohydrate lactone is 6-azido-6-deoxy-D-glucono-1,5-lactone. In some embodiments, the handle-substituted carbohydrate lactone has a purity of at least 85%, optionally, as determined by 1D ¹H-, 1D ¹³C-, and/or 2D ¹H-¹³C HSQC NMR. In some embodiments, the purity is at least 95%.

In a further aspect, the present disclosure provides an azido-substituted six-membered 1,5-carbohydrate-lactone with a purity of at least 85%, optionally, as determined by 1D ¹H-, 1D ¹³C-, and/or 2D ¹H-¹³C HSQC NMR. In some embodiments, the purity is at least 95%.

The present disclosure provides the following items:

[1] A method for synthesizing a handle-substituted carbohydrate lactone comprising contacting a handle-substituted aldose with a catalyst under aprotic conditions in the presence of a hydrogen acceptor.

[2] The method of item [1], wherein the handle-substituted carbohydrate lactone is an azido-substituted six-membered 1,5-carbohydrate-lactone, and the handle-substituted aldose is an azido-substituted pyranose.

[3] The method of item [2], wherein the catalyst is Shvo's catalyst.

[4] The method of any one of items [2]-[3], wherein the hydrogen acceptor is a compound comprising a ketone, carbonyl, alkene, alkyne and/or imine group.

[5] The method of any one of items [2]-[4], wherein the hydrogen acceptor is cyclohexanone.

[6] The method of any one of items [2]-[5], wherein the azido-substituted pyranose is according to formula (I):

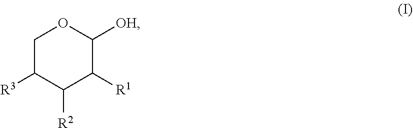

wherein:
(i) R¹, R² and R³ are each independently hydrogen, a hydroxyl, a methyl, or an azide,
(ii) at least one of R¹, R² and R³ is an azide, and
(iii) one or none of R¹, R² and R³ is a methyl.

[7] The method of any one of items [2]-[5], wherein the azido-substituted pyranose is according to formula (II):

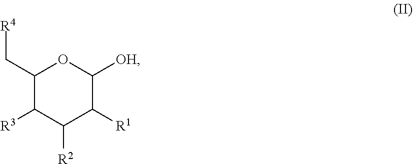

wherein:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide, and
(iv) one or none of R¹, R², R³ and R⁴ is a methyl.

[8] The method of any one of items [2]-[5], wherein the azido-substituted pyranose is according to formula (III):

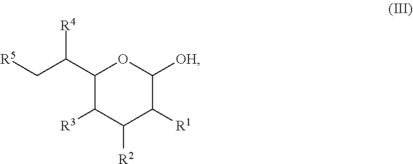

wherein:
(i) R¹, R² and R³ are each independently hydrogen, a hydroxyl, a methyl, or an azide,
(ii) R⁴ and R⁵ are each independently hydrogen, a hydroxyl, a methyl, an azide, or a carboxyl,
(iii) at least one of R¹, R², R³, R⁴ and R⁵ is an azide, and
(iv) one or none of R¹, R², R³, R⁴ and R⁵ is a methyl.

[9] The method of item [7], wherein the azido-substituted pyranose is 6-azido-6-deoxy-D-glucose, 4-azido-4-deoxy-D-glucose or 3-azido-3-deoxy-D-glucose.

[10] A composition comprising a handle-substituted carbohydrate lactone obtained or obtainable by the method of any one of items [1]-[9].

Compositions Comprising an N-Terminally Acylated and/or Conjugated Protein

In a further aspect, the present disclosure provides a composition comprising an N-terminally acylated protein, wherein the N-terminally acylated protein comprises formula (VII), formula (VIII) or formula (IX):

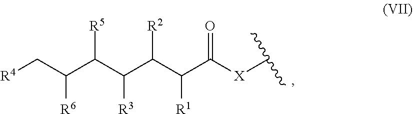

wherein:
(i) R¹, R², R³, R⁵ and R⁶ are each independently hydrogen, a hydroxyl, a methyl, or a handle,
(ii) R⁴ is hydrogen, a hydroxyl, a methyl, a handle, or a carboxyl,
(iii) at least one of R¹, R², R³ and R⁴ is a handle,
(iv) one or none of R¹, R², R³ and R⁴ is a methyl; and
(v) X comprises the N-terminal amino acid residue of the protein, wherein, optionally, the N-terminal amino acid residue is a Gly, Ala, Ser or His residue;

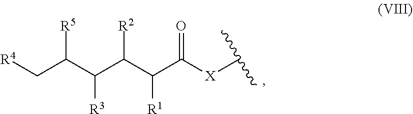

wherein:
(i) R¹, R², R³ and R⁵ are each independently hydrogen, a hydroxyl, a methyl, or a handle,
(ii) R⁴ is hydrogen, a hydroxyl, a methyl, a handle, a carboxyl, an ester or an amide,
(iii) at least one of R¹, R², R³ and R⁴ is a handle,
(iv) one or none of R¹, R², R³ and R⁴ is a methyl; and
(v) X comprises the N-terminal amino acid residue of the protein, wherein, optionally, the N-terminal amino acid residue is a Gly, Ala, Ser or His residue;

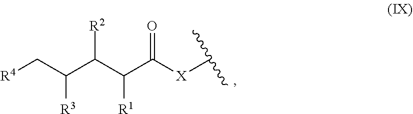

wherein:
(i) R¹, R² and R³ are each independently hydrogen, a hydroxyl, a methyl, or a handle,
(ii) R⁴ is a hydroxyl,
(iii) at least one of R¹, R² and R³ is a handle,
(iv) one or none of R¹, R² and R³ is a methyl; and
(v) X comprises the N-terminal amino acid residue of the protein, wherein, optionally, the N-terminal amino acid residue is a Gly, Ala, Ser or His residue. Preferably, the N-terminal amino acid residue of the protein is a Gly.

In some embodiments, at least 10% of the proteins in the composition comprise formula (VII), formula (VIII) or formula (IX) as described above. Preferably, at least 20% of the proteins in the composition comprise formula (VII), formula (VIII) or formula (IX) as described above. This may be determined using MALDI-TOF MS or QTOF MS.

In some embodiments, the handle is an Azide, Isothiocyanate, Isocyanate, Acyl azide, NHS ester, in situ NHS ester, Hydroxybenzotriazole (HOBt) ester, Sulfonyl chloride, -flouride, Sulfonyl ester (e.g., tosyl, mesyl, trifyl, tresyl esters etc.), Aldehyde, ketone, dicarbonyl, Aldehyde, Amine, Epoxide, Carbonate (e.g., succinimidyl carbonate), Cyclic imidocarbonate, NHS carbonate, N,N′-disuccinimidyl carbonate (DSC), Cyanate ester, Carbamate (e.g., imidazole carbamate), Acyl imidazole, NHS carbamate, Aryl halide (e.g., fluorobenzene derivatives), Haloacetyl or alkyl halide, Imidoester (imidates), Carboxylate, Acid anhydride, Fluorophenyl ester (pentafluorophenyl/PFP, tetrafluorophenyl/TFP, sulfo-tetrafluorophenyl/STP esters), Tris(hydroxymethyl)phosphine/THP, beta-[tris(hydroxymethyl)phospino]proprionic acid/THPP, Cyclic hemiacetal, Azlactone, Activated double bond, Methylpyridinium ether, 6-sulfo-cytosine derivative, activated halogen, haloacetyl derivative (e.g., iodoacetyl), benzyl halide, and alkyl halide (N- and S-mustards) derivative, Activated double bond, Vinyl sulfone, Maleimide, Aziridine, Aryl halide, Acryloyl derivative (e.g., acrylic and methacrylic acid derivatives), Disulfide (e.g., protected thiol, such as, pyridyl disulfide deriv., 5-thiol-2-nitrobenzoic acid, TNB-thiol—obtainable by treating a sulfhyrdyl with Ellman's reagent), Thiol (free), Thioester (e.g., phenylthioester), Cysteine attached via C-terminus, Cisplatin derivative, 2-cyanobenzothiazole, Diazoalkane compound, diazoacetyl compound, Epoxide, Aryl azide (e.g., phenyl azide, perfluorinated phenyl azide), Benzophenone, Anthraquinone, Diazo compound (e.g., diazotrifluoropropionate, diazopyruvate), Diazirene derivative, Psoralen compound, Halogenated phenyl azide, Alkene, Diene, Furan, Nitrone, Tetrazine, nitrile oxide, diazoalkane, syndone, quadricyclane, Boronic acid (e.g., phenyl boronic acid, and phenyldiboronic acid), BCN, BCN, 1,3-Dithiolium-4-olate, phosphine (e.g., triphenyl phosphine with electrophilic trap (Staudinger ligation)), phosphine derivative (e.g., bi- or tri-phenyl aryl ester, or acyl imidazole for traceless Staudinger ligation)), alkene, alkyne, strained alkyne, OCT, MOFO, DIFO, DIFO2, DIFO3, DIMAC, DIBO, DIBAC (ADIBO), BARAC, BCN, Sondheimer diyene, TMDIBO, S-DIBO, COMBO, PYRROC, TMTH, DIFBO, ALO, isonitrile, thioalkyne, keto-DIBO, strained olefin, trifluoromethyl-substituted oxanorbornadiene, Biotin, biotin derivative, boronic acid, p-boronophenylalanine, strained dialkyne or Sondheimer dialkyne. In a preferred embodiment, the handle is an azide, diazotrifluoropropionate, diazopyruvate), diazoalkane, isonitrile (aka isocyanide, carbylamine), boronic acid, p-boronophenylalanine, Amine, Aldehyde, ketone, dicarbonyl (i.e., phenylglyoxal deriv.), Aldehyde, Vinyl sulfone, Maleimide, Acryloyl derivative (e.g., acrylic and methacrylic acid derivative), Disulfide (e.g., protected thiol, such as, pyridyl disulfide deriv., 5-thiol-2-nitrobenzoic acid, TNB-thiol), Thioester (e.g., phenylthioester (on C-terminal peptides for native chemical ligation)), 2-cyanobenzothiazole, Alkene, Diene, Furan, Nitrone, Tetrazine, Tetrazine, nitrile oxides, syndone, quadricyclane, BCN, BCN, 1,3-Dithiolium-4-olate, phosphine (e.g., triphenyl phosphine with electrophilic trap), phosphine derivative (bi- or tri-phenyl aryl ester, acyl imidazole for traceless Staudinger ligation), alkene, alkyne, strained alkyne, OCT, MOFO, DIFO, DIFO2, DIFO3, DIMAC, DIBO, DIBAC (ADIBO), BARAC, BCN, Sondheimer diyene, TMDIBO, S-DIBO, COMBO, PYRROC, TMTH, DIFBO, ALO, thioalkyne, keto-DIBO, strained olefin, trifluoromethyl-substituted oxanorbornadiene, Biotin or biotin derivative. In a more preferred embodiment, the handle is an azide, Aldehyde, ketone, dicarbonyl (e.g., phenylglyoxal deriv.), Vinyl sulfone, Maleimide, Acryloyl derivative (e.g., acrylic and methacrylic acid derivative), Disulfide (e.g., protected thiol, such as, pyridyl disulfide deriv., 5-thiol-2-nitrobenzoic acid, TNB-thiol), thioester (e.g., phenylthioester), 2-cyanobenzothiazole, Alkene, Diene, Furan, Nitrone, Tetrazine, nitrile oxide, syndone, quadricyclane, BCN, BCN, 1,3-Dithiolium-4-olate, phosphine (e.g., triphenyl phosphine with electrophilic trap), phosphine derivative (bi- or tri-phenyl aryl ester, acyl imidazole), alkene, alkyne, strained alkynes, OCT, MOFO, DIFO, DIFO2, DIFO3, DIMAC, DIBO, DIBAC, BARAC, BCN, Sondheimer diyene, TMDIBO, S-DIBO, COMBO, PYRROC, TMTH, DIFBO, ALO, thioalkyne, keto-DIBO, strained olefin, trifluoromethyl-substituted oxanorbornadiene, Biotin or biotin derivative. Most preferably, the handle is an azide.

In some embodiments, the present disclosure provides a composition comprising an N-terminally acylated protein, wherein the N-terminally acylated protein comprises formula (IV):

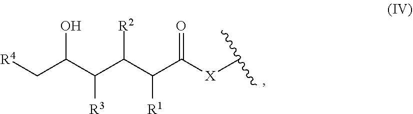

wherein:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide,
(iv) one or none of R¹, R², R³ and R⁴ is a methyl; and
(v) X comprises the N-terminal amino acid residue of the protein, wherein, optionally, the N-terminal amino acid residue is a Gly, Ala, Ser or His residue. Preferably, the N-terminal amino acid residue of the protein is a Gly.

In some embodiments, the present disclosure provides a composition comprising an N-terminally acylated protein, wherein the N-terminally acylated protein comprises formula (X):

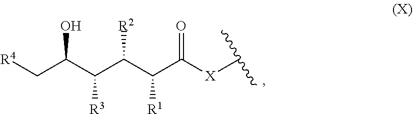

wherein:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide,
(iv) one or none of R¹, R², R³ and R⁴ is a methyl; and
(v) X comprises the N-terminal amino acid residue of the protein, wherein, optionally, the N-terminal amino acid residue is a Gly, Ala, Ser or His residue. Preferably, the N-terminal amino acid residue of the protein is a Gly.

In some embodiments, X comprises the amino acid sequence Gly-Gly or Gly-Ala.

In some embodiments, the protein comprises a His-tag at the N-terminus. This embodiment encompasses proteins wherein the first 3-6 amino acid residues at the N-terminus are His as well as proteins wherein 1-4 residues precede the His tag such as, for example, GHHHHHH and GSSHHHHHH. In some embodiments, X comprises 3-10 His residues.

In some embodiments, less than 5% of the total acylated protein is double acylated. In some embodiments, less than 3% of the total acylated protein is double acylated. This may be determined using matrix-assisted laser desorption/ionization time-of-flight mass spectrometry (MALDI-TOF MS) or Quadrupole time-of-flight mass spectrometry (QTOF MS). The extent of double acylation can be estimated as the ratio of the ion count (IC), also ion current, peak values for double acylated protein divided by the sum of all IC value for both acylated (mono and double) and non-acylated protein, that could participate in the reaction from MS data. MS data may be deconvoluted or used directly. The ratio could also be calculated by chromatographic separation by using carefully optimized ion-exchange protocol that can resolve individual charge variants of the molecule of interest by integrating the area under curves for the respectively mono- di- or otherwise acylated compounds as well as non-acylated species and calculated as above

In some embodiments, R⁴ is an azide, R³ is a hydroxyl, R² is a hydroxyl and R¹ is a hydroxyl. In some embodiments, R⁴ is a hydroxyl, R³ is an azide, R² is a hydroxyl and R¹ is a hydroxyl. In some embodiments, R⁴ is a hydroxyl, R³ is a hydroxyl, R² is an azide and R¹ is a hydroxyl. In some embodiments, R⁴ is a hydroxyl, R³ is a hydroxyl, R² is a hydroxyl and R¹ is an azide.

In some embodiments, X comprises the amino acid sequence Gly-Xaa¹-Xaa²-Xaa³, and
(i) Xaa¹ is an amino acid residue or absent;
(ii) Xaa² is an amino acid residue or absent; and
(iii) Xaa³ consists of 3 or more His residues. In some embodiments, Xaa¹ and Xaa² are each independently Ser, Gly or Ala. In some embodiments, Xaa¹ is Ser and Xaa² is Ser.

In some embodiments, X comprises the amino acid sequence GSSHHHHHH or GHHHHHH.

In some embodiments, X comprises the amino acid sequence Gly-Xaa¹-Xaa², and
(i) Xaa¹ is Gly, Ser or Ala; and
(ii) Xaa² is:
   TYSDH,
   TYSAH,
   TYSCH,
   KWSKR, or
   SGSK.

In some embodiments, X comprises the amino acid sequence GGTYSDH, GGTYSCH, GGKWSKR, or GASGSK.

In some embodiments, X comprises the amino acid sequence Xaa¹-Xaa²-Xaa³-Xaa⁴, and
(i) Xaa¹ is an amino acid residue selected from: Ala, Gly, Ser, His, or Leu;
(ii) Xaa² is any amino acid residue or absent;
(iii) Xaa³ is any amino acid residue or absent; and
(iv) Xaa⁴ consists of 3 or more His residues.

In some embodiments, X comprises:
(A) the amino acid sequence Gly-Xaa¹-Xaa²-Xaa³, and
   (i) Xaa¹ is any amino acid residue or absent;
   (ii) Xaa² is an amino acid residue or absent; and
   (iii) Xaa³ consists of 3 or more His residues;
      or
(B) the amino acid sequence Gly-Xaa¹, and
   (i) Xaa¹ is an amino acid residue selected from Gly, Ala, or Ser.

In some embodiments, X comprises:
(A) the amino acid sequence Xaa¹-Xaa² and
   (i) Xaa¹ is amino acid residue Gly or absent; and
   (ii) Xaa² consists of 1 or more His residues;
(B) the amino acid sequence Gly-Xaa¹-Xaa², and
   (i) Xaa¹ is amino acid residue selected from Ser, Gly, Ala, Tyr, Leu, Arg, or absent; and
   (ii) Xaa² consists of 2 or more His residues; or
(C) the amino acid sequence Gly-Xaa¹-Xaa², and
   (i) Xaa¹ is Gly, Ser or Ala; and
   (ii) Xaa² is:

Thr-Tyr-Ser-Asp-His,
Thr-Tyr-Ser-Cys-His,
Thr-Tyr-Ser-Ala-His,
Lys-Trp-Ser-Lys-Arg,
or
Ser-Gly-Ser-Lys.

In some embodiments, X comprises the amino acid sequence G, H, GA, GG, GH, GI, GP, GV, SH, GAH, GAP, GGH, GHH, GGK, GGS, GGV, RGS, SYH, AHHH, GAAH, GASH, GHHH, GSAH, GSSH, SSYH, SYYH, VHHH, GAPTL, GHHHH, GHHHHH, LRFKFY, HHHHHH, GASGSKG, GGKWSKR, GGTYSDH, GHHHHHH, GLRFKFY, HLRFKFY, KHHHHHH, GHLRFKFY, GSLRFKFY, GSHHHHHH, GSHLRFKFY, GSSHHHHHH, RGSHHHHHH, SYYHHHHHH, A, AH, AHH, GL, GS, GGT, GAA, GAS, GHL, GLR, GSA, GSH, GSL, GSS, GHLR, GLRF, GAPT, GASG, GGKW, GGTY, GSHL, GSLR, GASGS, GGKWS, GGTYS, GHLRF, GLRFK, GSHLR, GSLRF, GSSHH, GASGSK, GGKWSK, GGTYSD, GHLRFK, GLRFKF, GSHLRF, GSLRFK, GSSHHH, GHLRFKF, GSHLRFK, GSLRFKF, GSSHHHH, GSHLRFKF, GSSSHHHHH, HL, HLR, HLRF, HLRFK, HLRFKF, L, LR, LRF, LRFK, LRFKF, R, RG, S, SY, SS, SSY, SYY, V, VH or VHH. Preferably, X comprises the amino acid sequence GG, GHH, GHHH, GHHHH, GHHHHH, GHHHHHH, GSSHHHHHH, GSHHHHHH, GASGSKG, GGKWSKR or GGTYSDH.

It is conceivable that by testing other N-terminal sequences, sequences that are equally or even more prone to the selective N-terminal modification as disclosed herein may be identified. Since the solution space is considerably large, even a small library of just 6 random amino acids would give rise to over 64 million (20\hat{ }6) possible sequence variants. Library sizes increase exponentially with the length of the sequences to be tested (20\hat{ }n, where n is the length of the peptide sequence to be tested). Nonetheless, it is conceivable that consecutive rounds of phage display, displaying N-terminal sequence variants or random libraries may be screened (Keeble et al. 2017). Other library generation and screening methods are known such as mRNA-, ribosome-, yeast-, bacterial-, or DNA display for example. Screening of large peptide arrays may also be performed to identify similarly or better reacting sequences (Steffen et al. 2017).

In some embodiments, the composition further comprises a metal cation. In some embodiments, the metal cation is a d-block element. In some embodiments, the metal cation is a transition metal cation, preferably a divalent transition metal cation. In some embodiments, the transition metal cation is a divalent Zn, Ni or Cu cation. A divalent Ni or Cu cation is preferred for the purpose of inhibiting the reverse reaction.

The inventors observed that the acylation reaction is reversible. In particular, only 27% of the peptide comprising GHHHHHH at the N-terminus remained acylated after 14 days at room temperature whereas 75% of peptides comprising GGTYSDH remained acylated under the same conditions. Thus, His tags influence the reversibility of the reaction.

Without being bound by theory, it is thought that a divalent metal cation can interact with a His residue and may inhibit the reverse reaction.

Thus, the addition of the metal cations to the composition is particularly preferred when the protein comprises a His-rich sequence, such as a His tag (e.g., at least two or more His residues at the N-terminus).

It is therefore also conceivable to employ metal ligands, in particular polyvalent metal ligands, that have higher affinities for oligo-histidine, several of which are known (Hauser and Tsien 2007). It could be reasonably expected that such higher affinity ligands would provide even better inhibition of the reverse reaction.

While many divalent metals bind to poly histidine sequences reversibly, oxidation of imidazole-bound Co(II) or Ru(II) is known to result in a dramatic increase of the binding strength (Ren, Bobst, and Kaltashov 2019). It is thus conceivable that these metals could be used to irreversibly block the Histidine mediated effect of the reversibility reaction upon first binding to the modified Histidine rich sequence at a suitable pH, optional removal of excess metal, and finally oxidation in place to lock the histidine groups, in no specific order. Such oxidized metals can be reasonably expected to inhibit the reverse reaction across a wider range of pH values and would also be able to exert their reversibility protective effect in the presence of excess chelating reagents.

It is also conceivable that certain metals ions bind directly to the hydroxyl residues of the acyl group, as has analogously been demonstrated for Gd³⁺ in combination with the gluconoyl hydroxyls of a low molecular weight gluconamide model compound (Dill et al. 1985). Therefore, addition of metal ions may also modulate the reversibility of non-His tagged acylated substances according to the disclosure.

Furthermore, without being bound to theory, both the polyols of the gluconoyl modification and the histidine residue(s) may be expected to interact with a given metal ion at the same time.

The inventors also observed that the reverse reaction can be inhibited or stopped completely if the composition is lyophilized or frozen. Thus, in some embodiments, the composition is lyophilized or frozen.

In some embodiments, the composition further comprises a diol-ester forming agent. The diol-ester forming agent may form an adduct with the N-terminally acylated protein as shown in the following exemplary reaction scheme:

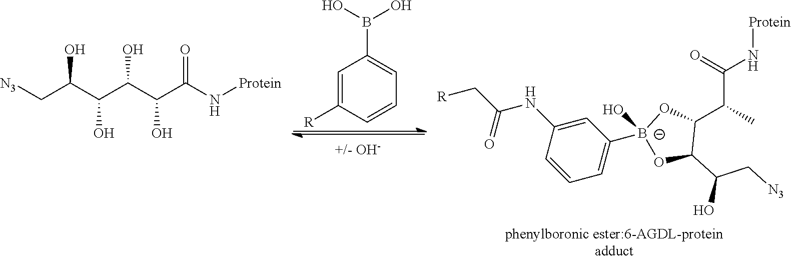

The inclusion of a diol-ester forming agent such as boric acid and/or boronic acid can also inhibit the reverse reaction. Thus, boric acid and boronic acid can function as a protective group for the acylated N-terminus. The protective effect is enhanced when the pH of the composition is 6 or higher. Thus, when boric and/or boronic acid is included in the composition, the pH of the solution should preferably be 5 or higher, more preferably 6 or higher to reduce the de-acylation rate of the protein.

Without being bound by theory, in principle any diol-ester forming agent may be used to inhibit, reduce or stop the reverse gluconoylation reaction. Besides boric acid, which preferably forms diols at slightly alkaline pH, it is known that binding of borate derivatives, even at pH values below 7.5. can be achieved by (i) phenylboronic acids substituted with electron-withdrawing groups, such as sulfonyl, fluoro, and carbonyl, on the phenyl ring; (ii) boronic acids containing intramolecular tetracoordinated B—N or B—O bonds (so-called Wulff-type); (iii) boronic acids containing intramolecular tricoordinated B—O bonds (known as “improved” Wulff-type); or (iv) heterocyclic boronic acids (Liu and He 2017). Therefore, any reagents from the aforementioned classes could provide the desired effect at a given pH value.

In some embodiments, the diol-ester forming agent has a pKa that is up to 2.2 pH units higher than the pH of the composition. For example, if the acylated protein is in a composition having a pH of 7, the pKa of the diol-ester forming agent may be 9.2 or lower.

In some embodiments, the diol-ester forming agent is or comprises Boric acid, Phenyl boronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib or Vaborbactam.

It is also conceivable that borinic acid ester forming reagents may inhibit the reverse reaction.

The acylated protein of any one of the above compositions may then be conjugated to another moiety using click chemistry (see Baskin & Bertozzi, 2010, Li & Zhang, 2016). Thus, the present disclosure also provides a composition comprising an N-terminally conjugated protein, wherein the N-terminally conjugated protein is obtained or obtainable by reacting the N-terminally acylated protein of the present disclosure with a compound comprising a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene).

The N-terminally conjugated protein may be the result of any one of the following reactions:

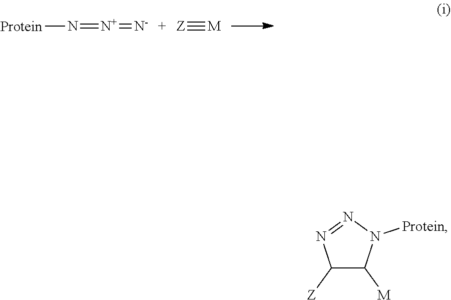

wherein Z is an aryl, alkyl or hydrogen and M is a desired conjugate;

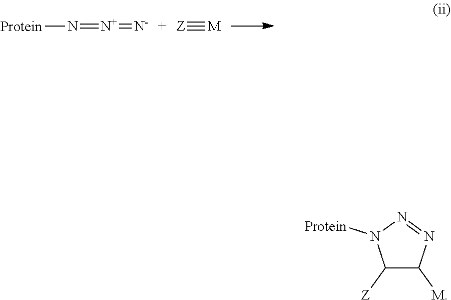

wherein Z is an aryl, alkyl or hydrogen and M is a desired conjugate;

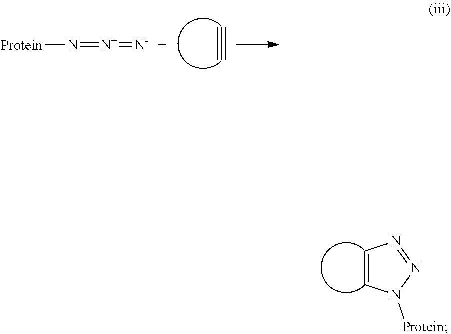

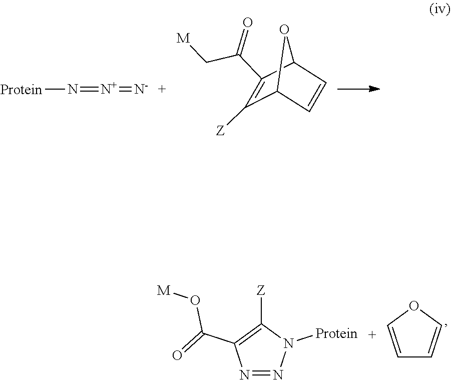

wherein M is a desired conjugate and Z is an electron withdrawing group (e.g., a trifluoromethylsulfonyl group, ammonium group, nitro group, sulfonic acid group, sulfonyl group, cyano group, trihalomethyl group, haloformyl group, formyl group, acyl group, carboxyl group or aminocarbonyl group); and

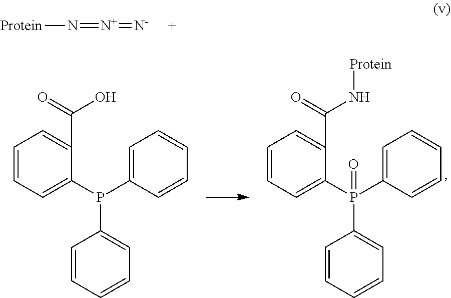

wherein any of the compounds of the aryl groups of the triarylphosphine may be covalently bound to a desired conjugate.

The desired conjugate may be 1-dehydrotestosterone, a chromophore, actinomycin D, aerogel, agar, agarose, alkaline phosphatase, alkylating agent, alumina gel, amino acid, amylopectin, anthracycline, antibiotic, antibody, antibody fragment, antigen, antimetabolite, avidin, bacteriophage, bead, beta-galactosidase, biochip, biofilm, biological cell, biotin, bromide, carbon nanotubes, cell membrane, cellular component, cellulose, Chemiluminescent compound, colchicin, contrast agent, cotton, cytochalasin B, cytolytic immunomodulatory proteins, daunorubicin, dendrimers, derivatized plastic film, dextran, diazocellulose, dihydroxy anthracin dione, DNA aptamer, DNA damaging inhibitors, doxorubicin, drug, elastine-like proteins and peptide, emetine, enzymatic substrate, enzyme, ethidium, etoposide, exosome, Extracellular Matrix scaffolds, FICOLL, firefly luciferase, fluorescent protein, fluorophore, free radical precursors, fullerenes, fuorescent lanthanide chelates, gamma emitting probes, glass bead, glass or magnetic support, glucocorticoid, glycan, glycogen, gramicidin D, hapten, heparin, hormone, horseradish peroxidase, hydrogel, IgG-binding, infrared emitting probes, inulin, ion chelating moiety, isobaric mass tags for mass spectrometry, isotopic mass tags for mass spectrometry, latex, layered material, lidocaine, lipid, lipid assembly, Liposomal spherical nucleic acids, liposome, magnetic bead, maleimide, mannan, mass tags for mass spectrometry, membrane, metal conducting, metal nonconducting, microfluidic chip, mithramycin, mitomycin, mitoxantrone, molecular scaffolds, multi-well plate, N-hydroxysuccinimide, nanocrystals, nanogel, nanoparticle, near infrared emitting probes, nitrocellulose, non-biological microparticle, nucleic acid, nucleoside, nucleotide, nylon, oligonucleotide, outer-membrane vesicle, PAMAM, paramagnetic bead, particle, PEG, PEP [a term describing the modification of proteins with synthetic polypeptides (also known as poly(α-amino acid)s)], peptide, phosphorescent dye, photosensitizer, phycobiliproteins, plastic bead, poly(acrylamide), poly(acrylate), polyethylene, polymer, polymeric membrane, polymeric microparticle, polyol, polypropylene, polysaccharide, polystyrene, polyvinylchloride, porous monoliths, procaine, propranolol, protein, psoralen, puromycin, quantum dots, radioisotope, radionuclides, resin, resonance probe, RNA aptamer, silica gel, silicon chip, spheroids, starch, streptavidin, superparamagnetic bead, surfaces, synthetic polymer, tandem dye, taxol, tenoposide, tetracaine, toxin, transcription inhibitor, tyramine, vesicle, vinblastine, vincristine, virus, Virus-like particles, or xenograft.

In some embodiments, the N-terminally acylated protein of the present disclosure is reacted with a compound according to formula E-L-M, wherein:
E comprises a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene);
L is a linker or absent; and
M is a desired conjugate.

In some embodiments, M is 1-dehydrotestosterone, a chromophore, actinomycin D, aerogel, agar, agarose, alkaline phosphatase, alkylating agent, alumina gel, amino acid, amylopectin, anthracycline, antibiotic, antibody, antibody fragment, antigen, antimetabolite, avidin, bacteriophage, bead, beta-galactosidase, biochip, biofilm, biological cell, biotin, bromide, carbon nanotubes, cell membrane, cellular component, cellulose, Chemiluminescent compound, colchicin, contrast agent, cotton, cytochalasin B, cytolytic immunomodulatory proteins, daunorubicin, dendrimers, derivatized plastic film, dextran, diazocellulose, dihydroxy anthracin dione, DNA aptamer, DNA damaging inhibitors, doxorubicin, drug, elastine-like proteins and peptide, emetine, enzymatic substrate, enzyme, ethidium, etoposide, exosome, Extracellular Matrix scaffolds, FICOLL, firefly luciferase, fluorescent protein, fluorophore, free radical precursors, fullerenes, fuorescent lanthanide chelates, gamma emitting probes, glass bead, glass or magnetic support, glucocorticoid, glycan, glycogen, gramicidin D, hapten, heparin, hormone, horseradish peroxidase, hydrogel, IgG-binding, infrared emitting probes, inulin, ion chelating moiety, isobaric mass tags for mass spectrometry, isotopic mass tags for mass spectrometry, latex, layered material, lidocaine, lipid, lipid assembly, Liposomal spherical nucleic acids, liposome, magnetic bead, maleimide, mannan, mass tags for mass spectrometry, membrane, metal conducting, metal nonconducting, microfluidic chip, mithramycin, mitomycin, mitoxantrone, molecular scaffolds, multi-well plate, N-hydroxysuccinimide, nanocrystals, nanogel, nanoparticle, near infrared emitting probes, nitrocellulose, non-biological microparticle, nucleic acid, nucleoside, nucleotide, nylon, oligonucleotide, outer-membrane vesicle, PAMAM, paramagnetic bead, particle, PEG, PEP [a term describing the modification of proteins with synthetic polypeptides (also known as poly(α-amino acid)s)], peptide, phosphorescent dye, photosensitizer, phycobiliproteins, plastic bead, poly(acrylamide), poly(acrylate), polyethylene, polymer, polymeric membrane, polymeric microparticle, polyol, polypropylene, polysaccharide, polystyrene, polyvinylchloride, porous monoliths, procaine, propranolol, protein, psoralen, puromycin, quantum dots, radioisotope, radionuclides, resin, resonance probe, RNA aptamer, silica gel, silicon chip, spheroids, starch, streptavidin, superparamagnetic bead, surfaces, synthetic polymer, tandem dye, taxol, tenoposide, tetracaine, toxin, transcription inhibitor, tyramine, vesicle, vinblastine, vincristine, virus, Virus-like particles, or xenograft.

In some embodiments, E is any one of the following compounds:

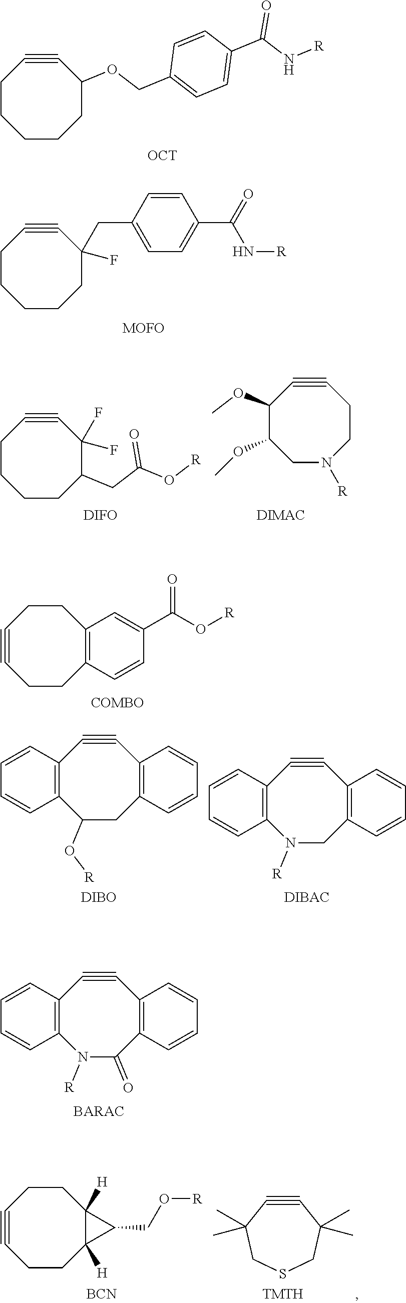

wherein R is L-M as described above.

In some embodiments, at least 10% of the proteins in the composition are conjugated. Preferably, at least 20% of the proteins in the composition are conjugated.

### Methods for Site-Specifically Modifying a Protein at the N-Terminus

In a further aspect the present disclosure provides a method for site-specifically modifying a protein (preferably at the N-terminus) comprising contacting a protein with a handle-substituted carbohydrate lactone. In some embodiments, the handle-substituted carbohydrate lactone has a five-, six- or seven-membered ring. In some embodiments, the carbohydrate lactone is a 1,5-lactone. In some embodiments, the carbohydrate lactone has a five-, six- or seven-membered ring and C6, C4 and/or C3 is substituted with a handle. The handle may in accordance with any one of the previous embodiments.

In some embodiments, the handle-substituted carbohydrate lactone is a lactone derivative of D-ribose, D-arabinose, D-xylose, D-lyxose, L-ribose, L-arabinose, L-xylose, or L-lyxose, D-allose, D-altrose, D-glucose, D-mannose, D-gulose, D-idose, D-galactose, D-talose, L-allose, L-altrose, L-glucose, L-mannose, L-gulose, L-idose, L-galactose, or L-Talose.

In some embodiments, the handle-substituted carbohydrate lactone is a lactone obtained or obtainable by a synthesis method of the present disclosure.

In some embodiments, the present disclosure provides a method for site-specifically modifying a protein at the N-terminus comprising contacting a protein with a compound according to formula (V):

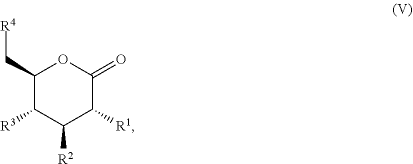

wherein:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide, and
(iv) one or none of R¹, R², R³ and R⁴ is a methyl.

In some embodiments, R⁴ is an azide, R³ is a hydroxyl, R² is a hydroxyl and R¹ is a hydroxyl. In some embodiments, R⁴ is a hydroxyl, R³ is an azide, R² is a hydroxyl and R¹ is a hydroxyl. In some embodiments, R⁴ is a hydroxyl, R³ is a hydroxyl, R² is an azide and R¹ is a hydroxyl. In some embodiments, R⁴ is a hydroxyl, R³ is a hydroxyl, R² is a hydroxyl and R¹ is an azide.

In some embodiments, the N-terminal amino acid residue is a Gly, Ala, Ser or His residue.

In some embodiments, the N-terminus comprises the amino acid sequence Gly-Gly or Gly-Ala.

In some embodiments, the protein comprises a His-tag at the N-terminus.

In some embodiments, the N-terminus of the protein comprises the amino acid sequence Gly-Xaa¹-Xaa²-Xaa³, wherein
(i) Xaa¹ is an amino acid residue or absent;
(ii) Xaa² is an amino acid residue or absent; and
(iii) Xaa³ consists of 3 or more His residues. In some embodiments, Xaa¹ and Xaa² are each independently Ser, Gly or Ala. In some embodiments, Xaa¹ is Ser and Xaa² is Ser.

In some embodiments, the N-terminus of the protein comprises the amino acid sequence GSSHHHHHH or GHHHHHH.

In some embodiments, the N-terminus of the protein comprises the amino acid sequence Gly-Xaa¹-Xaa², wherein
(i) Xaa¹ is Gly, Ser or Ala; and
(ii) Xaa² is:

TYSDH,
TYSAH
TYSCH,
KWSKR,
or
SGSK.

In some embodiments, the N-terminus of the protein comprises the amino acid sequence GGTYSDH, GGTYSCH, GGKWSKR, or GASGSK.

In some embodiments, the N-terminus of the protein comprises the amino acid sequence G, H, GA, GG, GH, GI, GP, GV, SH, GAH, GAP, GGH, GHH, GGK, GGS, GGV, RGS, SYH, AHHH, GAAH, GASH, GHHH, GSAH, GSSH, SSYH, SYYH, VHHH, GAPTL, GHHHH, GHHHHH, LRFKFY, HHHHHH, GASGSKG, GGKWSKR, GGTYSDH, GHHHHHH, GLRFKFY, HLRFKFY, KHHHHHH, GHLRFKFY, GSLRFKFY, GSHHHHHH, GSHLRFKFY, GSSHHHHHH, RGSHHHHHH, SYYHHHHHH, A, AH, AHH, GL, GS, GGT, GAA, GAS, GHL, GLR, GSA, GSH, GSL, GSS, GHLR, GLRF, GAPT, GASG, GGKW, GGTY, GSHL, GSLR, GASGS, GGKWS, GGTYS, GHLRF, GLRFK, GSHLR, GSLRF, GSSHH, GASGSK, GGKWSK, GGTYSD, GHLRFK, GLRFKF, GSHLRF, GSLRFK, GSSHHH, GHLRFKF, GSHLRFK, GSLRFKF, GSSHHHH, GSHLRFKF, GSSSHHHHH, HL, HLR, HLRF, HLRFK, HLRFKF, L, LR, LRF, LRFK, LRFKF, R, RG, S, SY, SS, SSY, SYY, V, VH or VHH. Preferably, the N-terminus of the protein comprises the amino acid sequence GG, GHH, GHHH, GHHHH, GHHHHH, GHHHHHH, GSSHHHHHH, GSHHHHHH, GASGSKG, GGKWSKR or GGTYSDH.

In some embodiments, the method further comprises adding a diol-ester forming agent. In some embodiments, the diol-ester forming agent is or comprises Boric acid, Phenyl boronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib or Vaborbactam. In some embodiments, the method further comprises adding a diol-ester forming agent. In some embodiments, the diol-ester forming agent is or comprises Boric acid, methylboronic acid, Phenyl boronic acid, 2-formylphenylboronic acid, 4-formylphenylboronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, 3-Aminophenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib or Vaborbactam.

The desired conjugate may be 1-dehydrotestosterone, a chromophore, actinomycin D, aerogel, agar, agarose, alkaline phosphatase, alkylating agent, alumina gel, amino acid, amylopectin, anthracycline, antibiotic, antibody, antibody fragment, antigen, antimetabolite, avidin, bacteriophage, bead, beta-galactosidase, biochip, biofilm, biological cell, biotin, bromide, carbon nanotubes, cell membrane, cellular component, cellulose, Chemiluminescent compound, colchicin, contrast agent, cotton, cytochalasin B, cytolytic immunomodulatory proteins, daunorubicin, dendrimers, derivatized plastic film, dextran, diazocellulose, dihydroxy anthracin dione, DNA aptamer, DNA damaging inhibitors, doxorubicin, drug, elastine-like proteins and peptide, emetine, enzymatic substrate, enzyme, ethidium, etoposide, exosome, Extracellular Matrix scaffolds, FICOLL, firefly luciferase, fluorescent protein, fluorophore, free radical precursors, fullerenes, fuorescent lanthanide chelates, gamma emitting probes, glass bead, glass or magnetic support, glucocorticoid, glycan, glycogen, gramicidin D, hapten, heparin, hormone, horseradish peroxidase, hydrogel, IgG-binding, immunomodulatory molecule, immunostimulatory molecule, immunosuppressive molecule, infrared emitting probes, inulin, ion chelating moiety, isobaric mass tags for mass spectrometry, isotopic mass tags for mass spectrometry, latex, layered material, lidocaine, lipid, lipid assembly, Liposomal spherical nucleic acids, liposome, magnetic bead, maleimide, mannan, mass tags for mass spectrometry, membrane, metal conducting, metal nonconducting, microfluidic chip, mithramycin, mitomycin, mitoxantrone, molecular scaffolds, multi-well plate, N-hydroxysuccinimide, nanocrystals, nanogel, nanoparticle, near infrared emitting probes, nitrocellulose, non-biological microparticle, nucleic acid, nucleoside, nucleotide, nylon, oligonucleotide, outer-membrane vesicle, PAMAM, paramagnetic bead, particle, PEG, PEP [a term describing the modification of proteins with synthetic polypeptides (also known as poly(α-amino acid)s)], peptide, phosphorescent dye, photosensitizer, phycobiliproteins, plastic bead, poly(acrylamide), poly(acrylate), polyethylene, polymer, polymeric membrane, polymeric microparticle, polyol, polypropylene, polysaccharide, polystyrene, polyvinylchloride, porous monoliths, procaine, propranolol, protein, psoralen, puromycin, quantum dots, radioisotope, radionuclides, resin, resonance probe, RNA aptamer, silica gel, silicon chip, spheroids, starch, streptavidin, superparamagnetic bead, surfaces, synthetic polymer, tandem dye, taxol, tenoposide, tetracaine, toxin, transcription inhibitor, tyramine, vesicle, vinblastine, vincristine, virus, Virus-like particles, or xenograft.

In some embodiments, the method further comprises adding a metal cation. In some embodiments, the metal cation is a d-block element. In some embodiments, the metal cation is a transition metal cation, preferably a divalent transition metal cation. In some embodiments, the transition metal cation is a divalent Zn, Ni, or Cu cation. A divalent Ni or Cu cation is preferred for the purpose of inhibiting the reversible reaction.

In some embodiments, the protein and compound according to formula (V) are contacted in an aqueous buffer. In some embodiments, the pH of the aqueous buffer is equal to or more than 4 and equal to or less than 9. In some embodiments, the pH of the aqueous buffer is equal to or more than 7 and equal to or less than 8. In some embodiments, the pH of the aqueous buffer is 7.5.

In some embodiments, the protein and compound according to formula (V) are contacted at a temperature of 4 to 37° C. In some embodiments, the reaction is allowed to proceed at a temperature of 4 to 37° C. It is conceivable to conduct the reaction below 4° C. but above freezing point of the respective reaction solution.

In some embodiments, the reaction may be allowed to proceed for equal to or more than about 30 minutes and less than or equal to about 24 hours. It is conceivable to conduct the reaction for a shorter time than 30 minutes if less acylation should be desirable.

In some embodiments, the method further comprises contacting the resulting acylated protein with a compound comprising a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene).

It is therefore clear to the skilled person that an azide can be used to conjugate a host of different functional components to the protein (see, for example, Spicer and Davis 2014, Baskin & Bertozzi, 2010, Li & Zhang, 2016).

In some embodiments, the resulting acylated protein is contacted with a compound according to formula E-L-M, wherein:
E comprises a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene);
L is a linker or absent; and
M is a desired conjugate.

In some embodiments, the handle-substituted carbohydrate lactone is reacted with a compound according to the formula E-L-M before allowing the resultant product to react with the protein, wherein:
E comprises a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene);
L is a linker or absent; and
M is a desired conjugate.

In some embodiments, M is 1-dehydrotestosterone, a chromophore, actinomycin D, aerogel, agar, agarose, alkaline phosphatase, alkylating agent, alumina gel, amino acid, amylopectin, anthracycline, antibiotic, antibody, antibody fragment, antigen, antimetabolite, avidin, bacteriophage, bead, beta-galactosidase, biochip, biofilm, biological cell, biotin, bromide, carbon nanotubes, cell membrane, cellular component, cellulose, Chemiluminescent compound, colchicin, contrast agent, cotton, cytochalasin B, cytolytic immunomodulatory proteins, daunorubicin, dendrimers, derivatized plastic film, dextran, diazocellulose, dihydroxy anthracin dione, DNA aptamer, DNA damaging inhibitors, doxorubicin, drug, elastine-like proteins and peptide, emetine, enzymatic substrate, enzyme, ethidium, etoposide, exosome, Extracellular Matrix scaffolds, FICOLL, firefly luciferase, fluorescent protein, fluorophore, free radical precursors, fullerenes, fuorescent lanthanide chelates, gamma emitting probes, glass bead, glass or magnetic support, glucocorticoid, glycan, glycogen, gramicidin D, hapten, heparin, hormone, horseradish peroxidase, hydrogel, IgG-binding, immunomodulatory molecule, immunostimulatory molecule, immunosuppressive molecule, infrared emitting probes, inulin, ion chelating moiety, isobaric mass tags for mass spectrometry, isotopic mass tags for mass spectrometry, latex, layered material, lidocaine, lipid, lipid assembly, Liposomal spherical nucleic acids, liposome, magnetic bead, maleimide, mannan, mass tags for mass spectrometry, membrane, metal conducting, metal nonconducting, microfluidic chip, mithramycin, mitomycin, mitoxantrone, molecular scaffolds, multi-well plate, N-hydroxysuccinimide, nanocrystals, nanogel, nanoparticle, near infrared emitting probes, nitrocellulose, non-biological microparticle, nucleic acid, nucleoside, nucleotide, nylon, oligonucleotide, outer-membrane vesicle, PAMAM, paramagnetic bead, particle, PEG, PEP [a term describing the modification of proteins with synthetic polypeptides (also known as poly(α-amino acid)s)], peptide, phosphorescent dye, photosensitizer, phycobiliproteins, plastic bead, poly(acrylamide), poly(acrylate), polyethylene, polymer, polymeric membrane, polymeric microparticle, polyol, polypropylene, polysaccharide, polystyrene, polyvinylchloride, porous monoliths, procaine, propranolol, protein, psoralen, puromycin, quantum dots, radioisotope, radionuclides, resin, resonance probe, RNA aptamer, silica gel, silicon chip, spheroids, starch, streptavidin, superparamagnetic bead, surfaces, synthetic polymer, tandem dye, taxol, tenoposide, tetracaine, toxin, transcription inhibitor, tyramine, vesicle, vinblastine, vincristine, virus, Virus-like particles, or xenograft.

In some embodiments, E is any one of the following compounds:

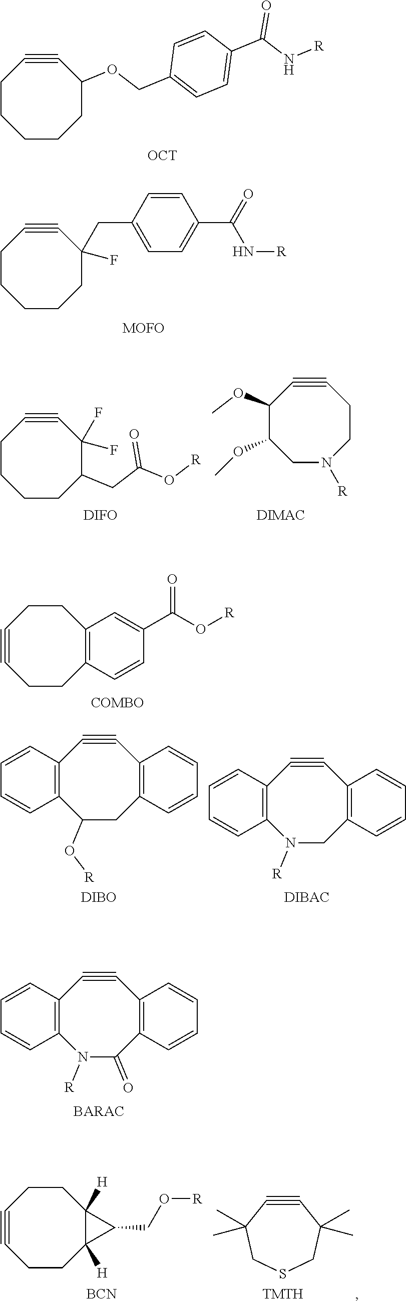

wherein R is L-M as described above.

In some embodiments, the compound according to formula (V) has a purity of at least 85%, preferably at least 95%. The purity may be determined using 1D ¹H-, 1D ¹³C-, and/or 2D ¹H-¹³C HSQC NMR.

In some embodiments the compound according to formula (V) is obtained or obtainable through a method according to the present disclosure.

In a further aspect, the present disclosure also provides a protein obtained or obtainable through any one of the methods of the present disclosure.

### Modulating the Rate of Hydrolysis

In a further aspect the present disclosure provides the use of a diol-ester forming agent and/or metal cation for modulating the hydrolysis rate of an acylated protein, preferably a N-terminally acylated protein according to the present disclosure. Without being bound to a particular theory, it is believed that the reverse reaction is a hydrolysis reaction.

The term “modulating” encompasses both promoting and inhibiting the hydrolysis rate because it is also envisioned that the concentrations of boric acid, boronic acid and/or metal cations can be decreased through dialysis or spin-filters in order to increase the rate of hydrolysis or the concentrations can be increased to inhibit the hydrolysis of the bond between the protein and the acyl group or the conjugate. Further, it is also envisioned that chelating agents can be used to sequester the metal cations and increase the hydrolysis rate. Ethylenediaminetetraacetic acid and citric acid are appropriate chelating agents for such an approach.

In some embodiments, the diol-ester forming agent is or comprises Boric acid, Phenyl boronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib or Vaborbactam. In some embodiments, the diol-ester forming agent is or comprises Boric acid, Phenyl boronic acid, 2-formylphenylboronic acid, 4-formylphenylboronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib or Vaborbactam.

In some embodiments, the metal cation is a d-block element. In some embodiments, the metal cation is a transition metal cation, preferably a divalent transition metal cation. In some embodiments, the transition metal cation is a divalent Zn, Ni, or Cu cation. A divalent Ni or Cu cation is preferred for the purpose of inhibiting the reversible reaction.

### Method for Trapping the Boron-Diol Ester

Having demonstrated the general possibility to trap both a 2-formylphenylboronic acid compound, and an N-hydroxylamine derivative onto the unmodified gluconylated- (see Example 19), and also onto a handle-substituted acylated peptide substrate (see Example 20), it appears conceivable to introduce (further additional, and/or orthogonal) reactive handle(s) to the N-terminus of a protein by introducing them via a 2-formylphenylboronic derivative, and/or an N-hydroxylamine derivative (Reaction scheme A). To this end, N-hydroxylamine-PEG derivatives are known in the art (Meadows et al., 2017). An N-terminally PEGylated protein substrate could be thus produced, but principally other handles could be incorporated into either N-hydroxylamine derivatives, and/or into derivatives of 2-formylphenylboronic acid or its benzoboroxloe tautomer. Methods to synthesize derivatives of 2-formylphenylboronic acid or its tautomer benzoboroxole are known in the art (Lulinski et al., 2007; Psurski et al., 2019; Kowalska et al., 2016). N-hydroxylamine derivative synthesis is also known in the art (Melman, 2010).

Reaction scheme A. Example of introduction of additional handles to an exemplary azido handle-substituted lactone treated substrate.

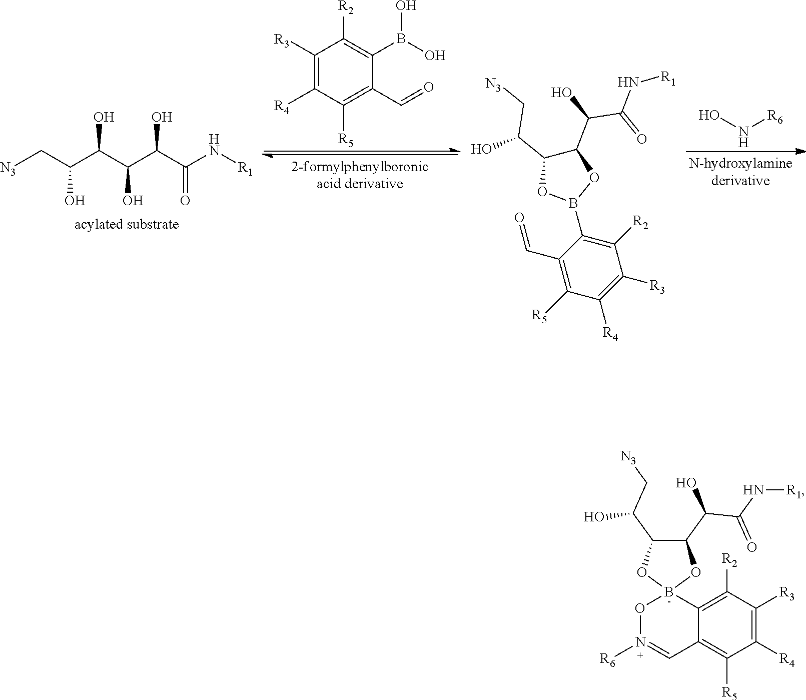

wherein, R1 is a protein, R2-6 independently additional handle(s) and/or hydrogens, and/or combinations thereof. In this case, only the 3,4-diol ester is shown although the formation of 2,3- and 4,5-diol esters are also conceivable. By carefully choosing the exact acylating reagent (carbohydrate lactone, or handle-substituted carbohydrate lactone) which allows the subsequent presence or absence, as well as location of (a) suitable hydroxyl(s) in the acylated substrate, other handle-adducts are obtainable.

In a further aspect the present disclosure provides the use of a diol-ester forming agent in combination with 2-formylphenylboronic acid and an N-hydroxylamine derivative, which allows to trap the diol-ester forming reagent for further modulating the hydrolysis rate of an acylated protein, preferably a N-terminally acylated protein according to the present disclosure.

An additional handle or additional handles may be introduced to the acylated peptides by means of combination of a handle-equipped 2-formylphenylboronic acid, and/or a handle-equipped N-hydroxylamine derivative to an acylated protein, or preferably N-terminally acylated protein according to the present disclosure.

The present disclosure also provides a method for labelling an N-terminally acylated protein, the method comprising contacting the protein with: (i) a 2-formylphenylboronic acid derivative and N-hydroxylamine, (ii) 2-formylphenylboronic acid and an N-hydroxylamine derivative, (iii) a 2-formylphenylboronic acid derivative and an N-hydroxylamine derivative or (iv) 2-formylphenylboronic acid and N-hydroxylamine. The N-terminally acylated protein may be an N-terminally acylated protein of the present disclosure or a naturally occurring N-terminally acylated protein, e.g., an N-terminally gluconoylated protein.

The 2-formylphenylboronic acid derivative and/or N-hydroxylamine derivative may be PEGylated.

The 2-formylphenylboronic acid derivative may be according to formula (XI):

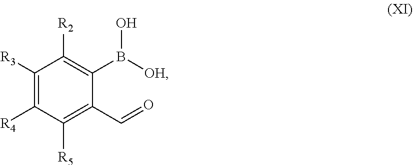

wherein each of R2, R3, R4 and R5 is, independently selected from a hydrogen or a handle and the derivative comprises at least one handle. In some embodiments, only one of R2, R3, R4 and R5 is a handle.

In any of the above embodiments, benzoboroxloe tautomers of the 2-formylphenylboronic acid or its derivative is used in addition to or instead of the 2-formylphenylboronic acid or its derivative.

The N-hydroxylamine derivative may be according to formula (XII):

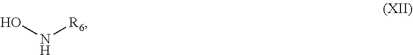

wherein R6 is a handle.

The present disclosure also provides a labeled protein obtained or obtainable by using any one of the above methods.

### Method for Identifying an Acylated Protein

The small Mw mass shift for successful GDL (178 Da) acylation can be observed using traditional Tricine-SDS-PAGE for small proteins (K. F. Geoghegan et al. 1999). However, monitoring installation of small Mw acyl groups becomes increasingly more difficult for bigger proteins using “low-tech” gel-based solutions. Cost-prohibitive mass spectrometric machinery such as MALDI-TOF or LC/MS may not be available in all standard molecular biology laboratories.

Thus, in a further aspect, the present disclosure provides a method for identifying an acylated protein according to the disclosure comprising running a sample suspected of containing an acylated protein on a diol-interacting, boron-containing acrylamide gel, optionally a methacrylamido phenylboronate acrylamide gel. Casting a methacrylamido phenylboronate acrylamide gel is as simple as adding an aqueous solution of 3-[(Methyl-)acrylamido]phenylboronic acid derivatives ((M)PBA) to the resolving SDS-PAGE gel mixture prior to polymerization (Pereira Morais et al. 2010).

Proteins which have been acylated with a, for example, six-membered 1,5-carbohydrate-lactone such as gluconolactone, or 6-azido-6-deoxy-glucono-1,5-lactone, can be distinguished from non-acylated proteins by running a control sample of the non-acylated protein alongside a sample suspected of containing an acylated protein. Acylated proteins experience a gel shift and tend to run at a higher apparent molecular weight than the non-acylated protein.

In some embodiments, other diol-ester forming agents may be immobilized in the SDS-PAGE gel mixture.

### Method for Purifying an Acylated Protein

In a further aspect, the present disclosure provides a method for purifying an acylated protein comprising: (1) binding a sample suspected of comprising the acylated protein onto a solid support comprising an immobilized diol-ester forming agent; and (2) eluting the protein. In some embodiments, the protein is eluted using a buffer with a pH of 6 or less and/or a buffer comprising competing diols. In some embodiments the protein is eluted isocratically, without changing the buffer composition, and is separated by elution volume/time.

In some embodiments, the diol-ester forming agent is Phenyl boronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, oxaborole, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib, or Vaborbactam. In some embodiments, the diol-ester forming agent is Phenyl boronic acid, 2-formylphenylboronic acid, 4-formylphenylboronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, oxaborole, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib, or Vaborbactam.

The term “competing diol” as used in the present application refers to a compound comprising at least two hydroxyl groups that binds to boronic acid and can dislodge the acylated protein from the solid support. The competing diol may be, for example, glycerol, Tris, a carbohydrate or a carbohydrate derivative.

In some embodiments, the method is performed using boronate affinity chromatography resin which may be packed into a column, centrifuge tube or a microplate. It is understood that alternative diol binding resins may be employed. For example, benzoboroxole resins can bind spiked glycosylated protein in PGL positive (that is non-gluconoylating) E. coli lysate at pH 7.4; i.e., not an E. coli B-strain (Rowe, El Khoury, and Lowe 2016). Due to the mode of benzoboroxole diol interaction (C. Chen et al. 2016), it is therefore conceivable that gluconoylated material should bind just as well, especially after an initial IMAC step. Employing benzoboroxole resin may also be beneficial as lower pH values are required compared to phenylboronic acid resins and may increase purity even further due to stronger interaction. A mixed mode affinity purification with IMAC and benzoboroxole purification is conceivable, as in addition to IMAC, benzoboroxole chemistry has also been demonstrated on monolith column format—possibly allowing higher flow rates compared to traditional agarose or PMMA support (H. Li et al. 2012).

In some embodiments, the method comprises one or more wash steps between step (1) and step (2). The wash steps are performed using a washing buffer which does not elute the acylated protein from the solid support to a significant extent but is able to remove at least some of the impurities from the sample. Thus, the wash step removes further impurities from the sample.

It follows that this method is also suitable for enriching the proportion of acylated protein in a sample and that such an embodiment is also encompassed by the present disclosure.

In some embodiments, the acylated protein is a N-terminally acylated protein according to the present disclosure.

In some embodiments, the protein is N-terminally acylated with a gluconoyl residue derived from contacting a suitable protein with glucono-1,5-lactone. Because non-target, host-cell derived proteins are not as susceptible to the gluconoyl modification, the target protein is preferentially modified and is thus susceptible to purification as described. This has the benefit that host-cell protein may be removed to give purer protein preparations.

In some embodiments the purified protein may be returned to its unacetylated form by incubating in conditions that allow reversibility of the reaction.

### Pharmaceutical Compositions

In a further aspect, the present disclosure provides a pharmaceutical composition comprising an acylated protein of the present disclosure and a pharmaceutically acceptable carrier and/or diluent. The pharmaceutical composition may comprise all or some of the components present in any one of the compositions described previously.

A pharmaceutical composition as described herein may also contain other substances. These substances include, but are not limited to, cryoprotectants, lyoprotectants, surfactants, bulking agents, anti-oxidants, and stabilizing agents. In some embodiments, the pharmaceutical composition may be lyophilized.

The term “cryoprotectant” as used herein, includes agents which provide stability against freezing-induced stresses. Cryoprotectants may also offer protection during primary and secondary drying and long-term product storage. Non-limiting examples of cryoprotectants include sugars, such as sucrose, glucose, trehalose, mannitol, mannose, and lactose; polymers, such as dextran, hydroxyethyl starch and polyethylene glycol; surfactants, such as polysorbates (e.g., PS-20 or PS-80); and amino acids, such as glycine, arginine, leucine, and serine. A cryoprotectant exhibiting low toxicity in biological systems is generally used.

In one embodiment, a lyoprotectant is added to a pharmaceutical composition described herein. The term “lyoprotectant” as used herein, includes agents that provide stability during the freeze-drying or dehydration process (primary and secondary freeze-drying cycles). This helps to minimize product degradation during the lyophilization cycle, and improve the long-term product stability. Non-limiting examples of lyoprotectants include sugars, such as sucrose or trehalose; an amino acid, such as monosodium glutamate, non-crystalline glycine or histidine; a methylamine, such as betaine; a lyotropic salt, such as magnesium sulfate; a polyol, such as trihydric or higher sugar alcohols, e.g., glycerin, erythritol, glycerol, arabitol, xylitol, sorbitol, and mannitol; propylene glycol; polyethylene glycol; pluronics; and combinations thereof. The amount of lyoprotectant added to a pharmaceutical composition is generally an amount that does not lead to an unacceptable amount of degradation when the pharmaceutical composition is lyophilized.

In some embodiments, a bulking agent is included in the pharmaceutical composition. The term “bulking agent” as used herein, includes agents that provide the structure of the freeze-dried product without interacting directly with the pharmaceutical product. In addition to providing a pharmaceutically elegant cake, bulking agents may also impart useful qualities in regard to modifying the collapse temperature, providing freeze-thaw protection, and enhancing the stability over long-term storage. Non-limiting examples of bulking agents include mannitol, glycine, lactose, and sucrose. Bulking agents may be crystalline (such as glycine, mannitol, or sodium chloride) or amorphous (such as dextran, hydroxyethyl starch) and are generally used in formulations in an amount from 0.5% to 10%.

Other pharmaceutically acceptable carriers, excipients, or stabilizers, such as those described in Remington's Pharmaceutical Sciences 16th edition, Osol, A. Ed. (1980) or Remington: The Science and Practice of Pharmacy 22^{nd} edition, Pharmaceutical press (2012), ISBN-13: 9780857110626 may also be included in a pharmaceutical composition described herein, provided that they do not adversely affect the desired characteristics of the pharmaceutical composition.

For solid pharmaceutical compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For solution for injection, the pharmaceutical composition may further comprise cryoprotectants, lyoprotectants, surfactants, bulking agents, anti-oxidants, stabilizing agents and pharmaceutically acceptable carriers. For aerosol administration, the pharmaceutical compositions are generally supplied in finely divided form along with a surfactant and propellant. The surfactant must, of course, be nontoxic, and is generally soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery. For suppositories, traditional binders and carriers may include, for example, polyalkalene glycols or triglycerides.

In a further aspect, the present disclosure provides a composition or pharmaceutical composition according to the present disclosure for use as a medicament.

In a further aspect, the present disclosure provides a method for administering a therapeutically effective amount of the composition or pharmaceutical composition according to the present disclosure to a subject or animal. In some embodiments, the method includes modulating the hydrolysis rate of the acylated and/or conjugated protein.

### Kit

In a further aspect, the present disclosure provides a kit comprising a protein and a handle-substituted carbohydrate lactone. In some embodiments, the handle-substituted carbohydrate lactone has a five-, six- or seven-membered ring. In some embodiments, the carbohydrate lactone is a 1,5-lactone. In some embodiments, the carbohydrate lactone has a five-, six- or seven-membered ring and C6, C4 and/or C3 is substituted with a handle. The handle may in accordance with any one of the previous embodiments.

In some embodiments, the handle-substituted carbohydrate lactone is a lactone derivative of D-ribose, D-arabinose, D-xylose, D-lyxose, L-ribose, L-arabinose, L-xylose, or L-lyxose, D-allose, D-altrose, D-glucose, D-mannose, D-gulose, D-idose, D-galactose, D-talose, L-allose, L-altrose, L-glucose, L-mannose, L-gulose, L-idose, L-galactose, or L-Talose.

In some embodiments, the handle-substituted carbohydrate lactone is a lactone obtained or obtainable by a synthesis method of the present disclosure.

In some embodiments, the present disclosure provides a kit comprising:
(i) a protein; and
(ii) a compound according to formula (V):

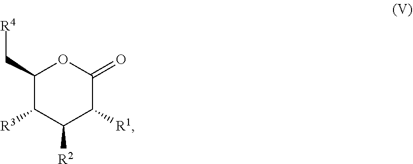

wherein:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide, and
(iv) one or none of R¹, R², R³ and R⁴ is a methyl.

In some embodiments, R⁴ is an azide, R³ is a hydroxyl, R² is a hydroxyl and R¹ is a hydroxyl. In some embodiments, R⁴ is a hydroxyl, R³ is an azide, R² is a hydroxyl and R¹ is a hydroxyl. In some embodiments, R⁴ is a hydroxyl, R³ is a hydroxyl, R² is an azide and R¹ is a hydroxyl. In some embodiments, R⁴ is a hydroxyl, R³ is a hydroxyl, R² is a hydroxyl and R¹ is an azide.

In some embodiments, the N-terminal amino acid residue of the protein is a Gly, Ala, Ser or His residue. In some embodiments, the N-terminus of the protein comprises the amino acid sequence Gly-Gly or Gly-Ala. In some embodiments, the protein comprises a His-tag at the N-terminus.

In some embodiments, N-terminus of the protein comprises the amino acid sequence Gly-Xaa¹-Xaa²-Xaa³, wherein
(i) Xaa¹ is an amino acid residue or absent;
(ii) Xaa² is an amino acid residue or absent; and
(iii) Xaa³ consists of 3 or more His residues. In some embodiments, Xaa¹ and Xaa² are each independently Ser, Gly or Ala. In some embodiments, Xaa¹ is Ser and Xaa² is Ser.

In some embodiments, the N-terminus of the protein comprises the amino acid sequence GSSHHHHHH or GHHHHHH.

In some embodiments, the N-terminus of the protein comprises the amino acid sequence Gly-Xaa¹-Xaa², and
(i) Xaa¹ is Gly, Ser or Ala; and
(ii) Xaa² is:

TYSDH,
TYSAH,
TYSCH,
KWSKR,
or
SGSK.

In some embodiments, the N-terminus of the protein comprises the amino acid sequence GGTYSDH, GGTYSCH, GGKWSKR, or GASGSK.

In some embodiments, the N-terminus of the protein comprises the amino acid sequence G, H, GA, GG, GH, GI, GP, GV, SH, GAH, GAP, GGH, GHH, GGK, GGS, GGV, RGS, SYH, AHHH, GAAH, GASH, GHHH, GSAH, GSSH, SSYH, SYYH, VHHH, GAPTL, GHHHH, GHHHHH, LRFKFY, HHHHHH, GASGSKG, GGKWSKR, GGTYSDH, GHHHHHH, GLRFKFY, HLRFKFY, KHHHHHH, GHLRFKFY, GSLRFKFY, GSHHHHHH, GSHLRFKFY, GSSHHHHHH, RGSHHHHHH, SYYHHHHHH, A, AH, AHH, GL, GS, GGT, GAA, GAS, GHL, GLR, GSA, GSH, GSL, GSS, GHLR, GLRF, GAPT, GASG, GGKW, GGTY, GSHL, GSLR, GASGS, GGKWS, GGTYS, GHLRF, GLRFK, GSHLR, GSLRF, GSSHH, GASGSK, GGKWSK, GGTYSD, GHLRFK, GLRFKF, GSHLRF, GSLRFK, GSSHHH, GHLRFKF, GSHLRFK, GSLRFKF, GSSHHHH, GSHLRFKF, GSSSHHHHH, HL, HLR, HLRF, HLRFK, HLRFKF, L, LR, LRF, LRFK, LRFKF, R, RG, S, SY, SS, SSY, SYY, V, VH or VHH. Preferably, X comprises the amino acid sequence GG, GHH, GHHH, GHHHH, GHHHHH, GHHHHHH, GSSHHHHHH, GSHHHHHH, GASGSKG, GGKWSKR or GGTYSDH.

In some embodiments, the kit further comprises a diol-ester forming agent. In some embodiments, In some embodiments, the diol-ester forming agent is or comprises Boric acid, Phenyl boronic acid, Nitrophenylboronic acid, Cyclopropylboronic acid, Cyclobutylboronic acid, 2-fluoro-5-nitrophenylboronic acid, Diphenylborinic acid, 2,6-bis(trifluoromethyl)phenylboronic, 4-(3-butenylsulfonyl) phenylboronic acid, Aminophenylboronic acid, vinylphenylboronic acid, 3-acrylamidophenylboronic acid, 2,4-difluoro-3-formyl-phenylboronic acid, 3-(dimethylaminomethyl) aniline-4-pinacol boronate, benzoxaborole, benzoxaborin, 3,3-dimethyl benzoxaborole, AN2898, SCYX-7158/AN5568, AN2718, AN3661, 3-carboxybenzoboroxole, pyridinylboronic acid, pyrimidineboronic acid, imdazoleboronic acid, isoxazoleboronic acid, diphenylborinic acid, Thienylboronic acid, Benzene-1,4-diboronic acid, phenyldiboronic (1,3-), Phenylethane Boronic Acid, 3-Nitrophenylboronic Acid, AN0128, Flovagatran, Talabostat, Delanzomib, Perboric acid, Dutogliptin, 4-Borono-L-phenylalanine, Tavaborole, Crisaborole, Bortezomib, Ixazomib or Vaborbactam.

In some embodiments, the kit further comprises a metal cation. In some embodiments, the metal cation is a d-block element. In some embodiments, the metal cation is a transition metal cation, preferably a divalent transition metal cation. In some embodiments, the transition metal cation is a divalent Zn, Ni, or Cu cation. A divalent Ni or Cu cation is preferred for the purpose of inhibiting the reversible reaction.

In some embodiments, the compound according to formula (V) has a purity of at least 85%, preferably at least 95%. The purity may be determined using 1D ¹H-, 1D ¹³C-, and/or 2D ¹H-¹³C HSQC NMR.

In some embodiments the compound according to formula (V) is obtained or obtainable through a method according to the present disclosure.

In some embodiments, the kit further comprises a compound comprising a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene).

In some embodiments, the kit further comprises a compound according to formula E-L-M, wherein:
E comprises a phosphine group (e.g., triphenylphosphine with electrophilic trap suitable for a Staudinger ligation), a phosphine derivative (e.g., bi- or tri-phenyl aryl ester, thioester, or acyl imidazole suitable for traceless Staudinger ligation), alkene group, alkyne group (suitable for CuAAC), strained alkyne group (suitable for SPAAC), OCT, MOFO, DIFO, DIFO2, DIFO3, DIMAC, DIBO, DIBAC (also known as ADIBO), BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group (e.g., trifluoromethyl-substituted oxanorbornadiene);
L is a linker or absent; and
M is a desired conjugate.

In some embodiments, M is 1-dehydrotestosterone, a chromophore, actinomycin D, aerogel, agar, agarose, alkaline phosphatase, alkylating agent, alumina gel, amino acid, amylopectin, anthracycline, antibiotic, antibody, antibody fragment, antigen, antimetabolite, avidin, bacteriophage, bead, beta-galactosidase, biochip, biofilm, biological cell, biotin, bromide, carbon nanotubes, cell membrane, cellular component, cellulose, Chemiluminescent compound, colchicin, contrast agent, cotton, cytochalasin B, cytolytic immunomodulatory proteins, daunorubicin, dendrimers, derivatized plastic film, dextran, diazocellulose, dihydroxy anthracin dione, DNA aptamer, DNA damaging inhibitors, doxorubicin, drug, elastine-like proteins and peptide, emetine, enzymatic substrate, enzyme, ethidium, etoposide, exosome, Extracellular Matrix scaffolds, FICOLL, firefly luciferase, fluorescent protein, fluorophore, free radical precursors, fullerenes, fuorescent lanthanide chelates, gamma emitting probes, glass bead, glass or magnetic support, glucocorticoid, glycan, glycogen, gramicidin D, hapten, heparin, hormone, horseradish peroxidase, hydrogel, IgG-binding, infrared emitting probes, inulin, ion chelating moiety, isobaric mass tags for mass spectrometry, isotopic mass tags for mass spectrometry, latex, layered material, lidocaine, lipid, lipid assembly, Liposomal spherical nucleic acids, liposome, magnetic bead, maleimide, mannan, mass tags for mass spectrometry, membrane, metal conducting, metal nonconducting, microfluidic chip, mithramycin, mitomycin, mitoxantrone, molecular scaffolds, multi-well plate, N-hydroxysuccinimide, nanocrystals, nanogel, nanoparticle, near infrared emitting probes, nitrocellulose, non-biological microparticle, nucleic acid, nucleoside, nucleotide, nylon, oligonucleotide, outer-membrane vesicle, PAMAM, paramagnetic bead, particle, PEG, PEP [a term describing the modification of proteins with synthetic polypeptides (also known as poly(α-amino acid)s)], peptide, phosphorescent dye, photosensitizer, phycobiliproteins, plastic bead, poly(acrylamide), poly(acrylate), polyethylene, polymer, polymeric membrane, polymeric microparticle, polyol, polypropylene, polysaccharide, polystyrene, polyvinylchloride, porous monoliths, procaine, propranolol, protein, psoralen, puromycin, quantum dots, radioisotope, radionuclides, resin, resonance probe, RNA aptamer, silica gel, silicon chip, spheroids, starch, streptavidin, superparamagnetic bead, surfaces, synthetic polymer, tandem dye, taxol, tenoposide, tetracaine, toxin, transcription inhibitor, tyramine, vesicle, vinblastine, vincristine, virus, Virus-like particles, or xenograft.

In some embodiments, E is any one of the following compounds:

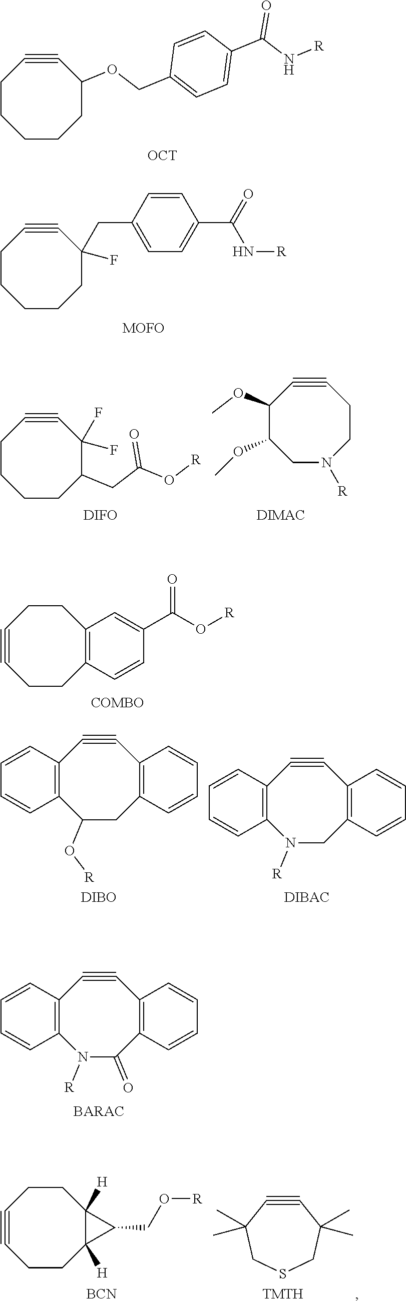

wherein R is L-M as described above.

### Method for Measuring the Extent of Acylation

In one aspect, the present disclosure provides a method for measuring the extent of protein acylation in a sample comprising adding a diol-ester forming agent to the sample. The diol-ester forming agent may be any diol-ester forming agent previously mentioned.

The method may further involve digesting the proteins in the sample using an enzyme and then performing mass spectrometry on the sample. The enzyme may be trypsin.

In some embodiments, the method is used for determining the extent of gluconoylation and/or phospho-gluconoylation.

### Vaccine

In another aspect, the present disclosure provides a vaccine comprising the N-terminally acylated protein of the present disclosure. In some embodiments, the N-terminally acylated protein is an antigen. In some embodiments, the antigen is conjugated to a VLP (virus-like particle, see Yan et al. 2015). In another embodiment, the N-terminally acylated protein is a carrier protein, such as the monomer of a VLP, and the N-terminally acylated protein is conjugated to an antigen.

Conjugation between two proteins, for example, a VLP and an antigen, may occur via click chemistry. For example, the VLP may be reacted with a heterobifunctional crosslinker comprising a reactive group that reacts with an amino group and a reactive group that reacts with an azide group. The antigen may be reacted with an azide-substituted carbohydrate lactone. The activated VLP and antigen may then be contacted to produce a conjugate. An exemplary reaction is shown in Example 17. In an alternative embodiment, the VLP is reacted with the azide-substituted carbohydrate lactone and the antigen is reacted with the heterobifunctional crosslinker.

In some embodiments, the heterobifunctional crosslinker comprises a bicyclo[6.1.0]nonyne (BCN) group and a N-hydroxysuccinimide (NHS) group. The heterobifunctional crosslinker may further comprise a linker which covalently links the two chemical groups. The linker may for example, be a PEG moiety such as PEG8. In some embodiments, the heterobifunctional crosslinker is NHS-PEG8-BCN as used in Example 17.

In some embodiments, the azide-substituted carbohydrate lactone is 6-azido-6-deoxy-D-glucono-1,5-lactone.

In some embodiments, the antigen is derived from a coronavirus (e.g., SARS-CoV-2). In some embodiments, the antigen is or comprises the receptor binding domain of SARS-CoV-2 (e.g., SEQ ID NO: 91) or an antigenic fragment thereof. In some embodiments, the antigen is or comprises SEQ ID NO: 91 or a sequence that has at least 75, 80, 85, 90, 95, 98 or 99% sequence identity with SEQ ID NO: 91. In some embodiments, the antigen is or comprises SEQ ID NO: 91 or a sequence that has at least 99% sequence identity with SEQ ID NO: 91. In some embodiments, the antigen may comprise a purification tag such as a His tag (see, for example, SEQ ID NO: 87).

(His tag-RBD)
SEQ ID NO: 87
GHHHHHHTNLCPFGEVFNATRFASVYAWNRKRISNCVADYSVLYNSASF
STFKCYGVSPTKLNDLCFTNVYADSFVIRGDEVRQIAPGQTGKIADYNY
KLPDDFTGCVIAWNSNNLDSKVGGNYNYLYRLFRKSNLKPFERDISTEI
YQAGSTPCNGVEGFNCYFPLQSYGFQPTNGVGYQPYRVVVLSFELLHAP
ATVCG
(RBD)
SEQ ID NO: 91
TNLCPFGEVFNATRFASVYAWNRKRISNCVADYSVLYNSASFSTFKCYG
VSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTP
CNGVEGFNCYFPLQSYGFQPTNGVGYQPYRVVVLSFELLHAPATVCG

In some embodiments, the VLP comprises a Qbeta coat protein (e.g., SEQ ID NO: 86). In some embodiments, the VLP comprises SEQ ID NO: 86 or a sequence that has at least 75, 80, 85, 90, 95, 98 or 99% sequence identity with SEQ ID NO: 86. In some embodiments, the VLP comprises SEQ ID NO: 86 or a sequence that has at least 99% sequence identity with SEQ ID NO: 86.

(Qbeta)
SEQ ID NO: 86
MAKLETVTLGNIGKDGKQTLVLNPRGVNPTNGVASLSQAGAVPALEKRV
TVSVSQPSRNRKNYKVQVKIQNPTACTANGSCDPSVTRQAYADVTFSFT
QYSTDEERAFVRTELAALLASPLLIDAIDQLNPAY

The present disclosure provides the vaccine, composition, N-terminally acylated protein, or handle-substituted carbohydrate lactone of the present disclosure for use as a medicament.

The handle-substituted carbohydrate lactone of the present disclosure may be for use in a method of eliciting an immune response in an individual or animal, wherein the handle-substituted carbohydrate lactone is an adjuvant. The vaccine, composition or N-terminally acylated protein of the present disclosure may be for use in a method of eliciting an immune response in an individual or animal.

The present disclosure provides the vaccine, composition, or N-terminally acylated protein of the present disclosure for use in a method of treating or preventing a coronavirus-related disorder. The coronavirus-related disorder may be SARS (severe acute respiratory syndrome), MERS (Middle East respiratory syndrome) and/or COVID-19 (coronavirus disease).

### EXAMPLES

### Example 1

### Synthesis of 6-Azido-6-Deoxy-D-Glucono-1,5-Lactone

### Chemical Synthesis

15 mg of 6-azido-6-deoxy-D-glucose (C₆H₁₁N₃O₅, CAS 20847-05-6; Carbosynth Ltd., Compton, UK, Cat. no. MA02620) were transferred into a conical glass flask together with 0.5 mg of Shvo's catalyst (Alfa Aesar, UK Cat. no. 46884) and 1 mL of cyclohexanone (Alfa Aesar cat. no. A15607.AP). The reaction mixture was degassed by passing nitrogen gas for 15 min and then sealed tightly. The degassed reaction mixture was kept overnight (18 h) constantly stirring at 50° C. A color change from light yellow to orange/brown was observed.

The reaction mixture was transferred to microcentrifuge tubes and spun for 15 min at 20,000 g, a dark brown precipitate of small size was observed at the bottom. The supernatant was transferred to a new tube and 3.5 volumes of hexane were added. A white precipitate was observed immediately. After carrying out the precipitation for 15 min at room temperature (RT), the reaction mixture was spun for 5 min at 20,000 g and the tight white pellet was observed at the bottom. After discarding the supernatant, the pellet was dried under 30° C., reduced pressure conditions. The dried pellet was redissolved in 5:1 mixture of ethyl acetate/acetone and the traces of remaining solids were precipitated by spinning for 20 min at 20,000 g. The supernatant was transferred to a new tube and spun a second time for 20 min at 20,000 g. The colorless liquid was evaporated under 30° C. reduced pressure conditions yielding amorphous white powdered solid with a yellow hint.

### Characterization

The melting point was determined on a Gallenkamp apparatus and the temperature was measured with a calibrated thermometer—the m.p. was 114-118° C. (uncorrected).

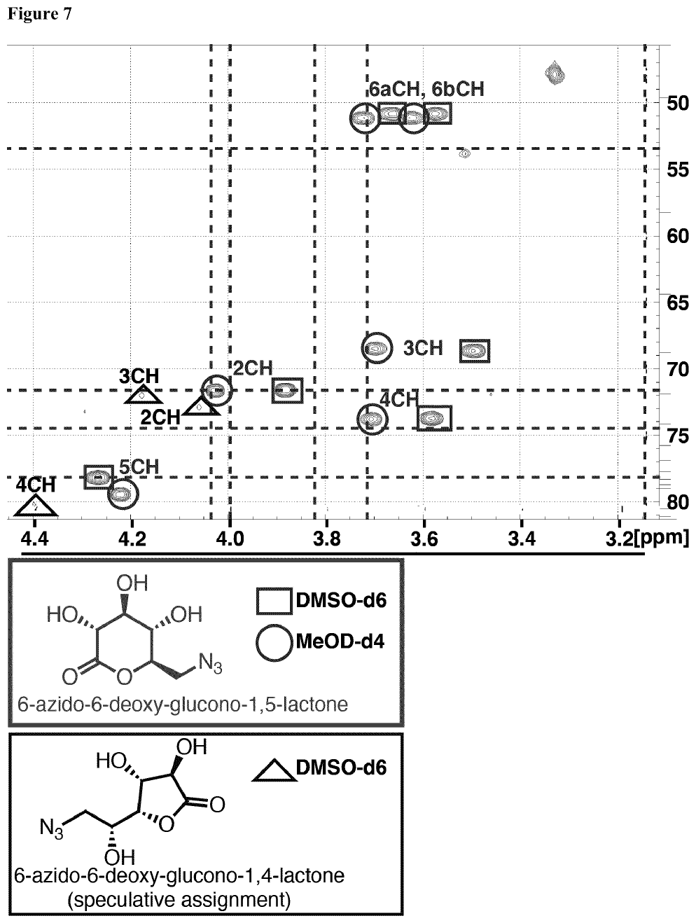

1D ¹H and ¹³C NMR, as well as 2D ¹H-¹³C HSQC NMR in DMSO-d6 was performed to compare the structure of the present compound against the reference spectra for glucono-1,5 and 1,4-lactone in the same solvent as described by Bierenstiehl 2004. Solution samples were prepared just prior to spectrum acquisition to reduce possible lactone isomerization effects. Sample was dissolved to a concentration of ˜1.3-1.6 mg/mL and spectra were obtained on an 800 MHz instrument with ultra-shielded magnet (Bruker, 800 US 2). DMSO-d6 data was obtained at 30° C. (controlled) to avoid freezing of the solvent. The acquisition time was kept purposefully short, in order to avoid possible isomerization effects in solution and allowed recording of the dominant carbon signals (C2-C6, FIG. 7). Where possible, J values are reported, otherwise J values were not accurately obtainable.

DMSO-d6: ¹H NMR (800 MHz, DMSO-d6): δ 4.26 (1H, m, J=2.6, 5.5, 9.4, assigned as H5); 3.90 (1H, dd, J=5.5, 8.1, 5.0, assigned as H2); 3.67 (1H, m, assigned as H6a), 3.57 (1H, m, J=13.5, 2.5, assigned as H6b); 3.60 (1H, m, assigned as H4); 3.50 (1H, m, assigned as H3). ¹³C NMR (DMSO-d6): δ 78.1 (assigned as C5), 73.8 (assigned as C4), 71.5 (assigned as C2), 68.6 (assigned as C3), 50.8 (assigned as C6). The C1 signal was not observed in this particular experiment due to short spectral acquisition time. A ¹H NMR signal at 3.3 in DMSO-d6 was observed and derives from residual H₂O.

### Data Analysis and Comparison Against References

Comparison of the major 2D ¹H-¹³C NMR signals obtained in DMSO-d6 to the spectra of both the non-azidated glucono-1,4- and the 1,5-lactone in the same solvent (Bierenstiel 2004) showed much better agreement with the latter. Non-azidated glucono-1,4- and 1,5-lactone ¹³C signals differ in particular in their C3, C4 and C5 values. The experimentally obtained C3, C4 and C5 ¹³C values for the azidated-compound were in much better agreement with the non-azidated glucono-1,5-lactone. This data provides strong evidence for the 6-azido-6-deoxy-glucono-1,5-lactone.

Whereas the C2, C3, C4 signals were relatively unaffected by introduction of the azide in position C6 compared to the non-azido glucono-1,5-lactone (Bierenstiehl 2004), minor shifts were observed for C5 and major shifts for C6 signal in both ¹H and ¹³C NMR spectra, as may be expected for a C6 substitution.

Interestingly, several minor 2D NMR signals were observed that, when compared to the non-azidated glucono-1,4-lactone (Bierenstiel 2004), provided a good fit for a speculative 6-azido-6-deoxy-glucono-1,4-lactone. In particular C2, C3, and C4 could be matched, whereas no obvious signals for C5 or C6 of a 1,4-lactone could be observed, suggesting that azidation is also affecting their shifts in the presumed 1,4-lactone. This observation suggests that small amounts of 6-azido-6-deoxy-glucono-1,4-lactone could be present post synthesis or that the 1,5-lactone can partially isomerize to the 1,4-lactone on the timescale of the experiment in solution.

The purity of the 6-azido-6-deoxy-glucono-1,5-lactone of this particular batch was estimated to be ˜88% pure by integration (DMSO-d6).

Since the structure of the disclosed compound was now elucidated, 2D ¹H-¹³C HSQC NMR analysis of the same material was performed in MeOD-d4 to better enable comparison to the bromine oxidation product of the azido-glucose in the same solvent (Chaveriat 2006), as well as comparison to the literature ¹H spectra of the non-azido glucono-1,4-lactone (Walaszek and Horton 1982).

Once more, solution samples were prepared just prior to spectrum acquisition to reduce possible lactone isomerization effects. Sample was dissolved in MeOH-d4 at a concentration of ˜1.3-1.6 mg/mL. MeOH-d4 spectra were obtained at 25° C. (controlled). For the 2D experiment, the acquisition time was kept purposefully short, in order to avoid possible isomerization effects in solution and allowed recording of the dominant carbon signals (C2-C6, FIG. 7). For another, separate 1D ¹³C NMR experiment, the acquisition time was extended to allow recording of C1.

6-azido-6-deoxy-glucono-1,5-lactone in solution:

MeOD (C2-C5): ¹H NMR (800 MHz, MeOD): δ 4.22 (1H, m, assigned as H5); 4.02 (1H, d, J=8.4, assigned as H2); 3.72 (1H, m, assigned as 6Ha); 3.62 (1H, m, assigned as 6Hb); 3.71 (1H, m, assigned as H4); 3.70 (1H, m, assigned as H3). ¹³C NMR (MeOD): δ 79.5 (assigned as C5), 73.8 (assigned as C4), 71.5 (assigned as C2), 68.4 (assigned as C3), 51.1 (assigned as C6). The ¹H NMR peaks 4.8 and 3.3 in MeOH-d4 are the residual H from the OH and CHD2.

¹³C NMR (MeOD, full 1D): δ 173.0 (assigned as C1), 80.8 (assigned as C5), 75.2 (assigned as C4), 73.1 (assigned as C2), 69.9 (assigned as C3), 52.6 (assigned as C6).

Due to absence of 2D NMR data in Chaveriat 2006, it is impossible to compare assigned ¹H, but careful ranked numerical comparison can still be performed. Detailed comparison of the ¹H signals from the 2D NMR experiment for the 6-azido-6-deoxy-glucono-1,5-lactone with Chaveriats 1D ¹H signals, revealed differences between the spectra. In particular, it was observed that that smallest chemical ¹H shift value of the compound disclosed here is bigger than the smallest chemical shift value reported by Chaveriat (3.62 vs. 2.97 ppm). The second lowest chemical shift obtained for the compound in this disclosure was larger than Chaveriat's second lowest one (3.70 vs. 3.15 ppm).

Of note, NMR spectra of glucono-1,5-lactone in DMSO-d6 were highly reproducible between different laboratories and instruments in the same solvent (cf. Bierenstiel 2004 and Walaszek 1982 for ¹³C, ¹H and J couplings). However, proton NMR was not reproducible for the present compound and Chaveriat's compound in MeOH-d4.

Once more, due to absence of 2D NMR data in Chaveriat 2006, it is impossible to compare assigned ¹³C shifts, but some assignments and comparison can still be performed. In particular, the C1 and the C6 value may be unambiguously assigned. The C1 carbonyl shifts are in very good agreement with each other, when comparing to the full 1D ¹³C spectrum. The ¹³C C6 value for the 2D NMR spectrum is 2.3 ppm, and the full 1D ¹³C C6 signal is 0.85 ppm lower when compared to Chaveriats C6 signal. The four remaining ¹³C signals for both the full and the ¹³C 2D NMRs could not be unambiguously matched.

In conclusion, the comparison of ¹H and ¹³C NMR spectra for the compound disclosed here with Chaveriat's spectrum does not provide unequivocal evidence that identical compounds have been obtained by aqueous bromine and Shvo oxidation methods.

To further characterize the compound here and allow additional comparison to additional glucono-1,5-lactone spectra, the compound disclosed here was also analyzed in D₂O by 1D ¹H-, 1D ¹³C-, 2D ¹H-¹³C HSQC, and additionally 2D ¹H-¹³C Heteronuclear Multiple Quantum Coherence (HMQC) NMR.

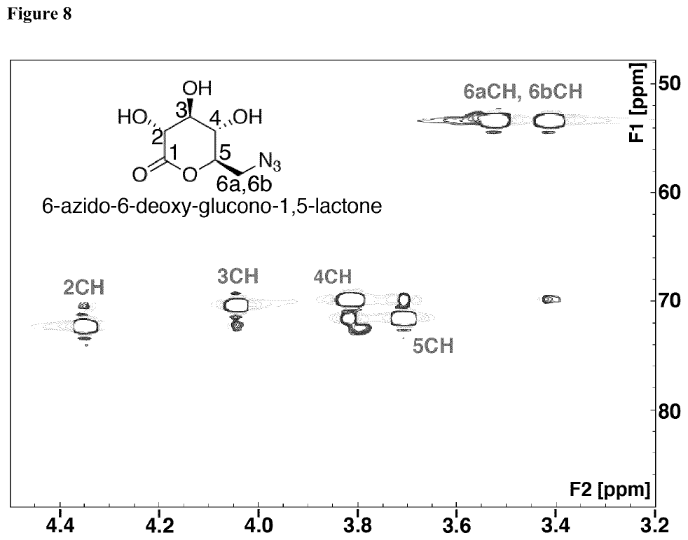

In D₂O, the 1D proton and 1D J-modulated ¹³C data showed six distinct proton environments and six distinct carbon environments in the molecule consistent with four methine, one methylene and one carbonyl group. Superimposition of the 2D ¹H-¹³C HSQC data (heteronuclear single quantum correlation), showing short range coupling, together with the chemical shifts observed allowed once more assignment of the major signals to the 6-azido-6-deoxy-glucono-1,5-lactone (FIG. 8).

The purity of this separate batch was determined to be >95% by integration of 1D ¹H NMR.

### Example 2

### Synthesis of 6-Azido-6-Deoxy-D-Glucono-1,5-Lactone (Crystalline Form)

15 mg of 6-azido-6-deoxy-D-glucose were put into a conical flask together with 2 mg of Shvo's catalyst and 1 mL of cyclohexanone. The reaction mixture was degassed by bubbling nitrogen gas for 15 min and then sealed tightly. The degassed reaction mixture was kept for 4 h constantly stirring at 45° C. A color change from light yellow to dark orange was observed.

The reaction mixture was spun for 15 min at 20,000 g and a dark brown precipitate of small size was observed at the bottom. The supernatant was transferred to a new tube and 3.5 volumes of hexane were added to the supernatant volume. Upon contact and mixing, a whitish precipitate was observed immediately. After carrying out the precipitation for 15 min at RT, the reaction mixture was spun for 5 min at 20,000 g and a tight white pellet was observed at the bottom of the microcentrifuge tube. The supernatant was discarded and the wet pellet was resuspended in 5:1 mixture of ethyl acetate/acetone by vortexing. Traces of remaining solids (red brownish color) were precipitated by spinning for 20 min at 20,000 g. The supernatant was transferred to a new tube and spun a second time for 20 min at 20,000 g. The supernatant, a colorless liquid, was transferred once more into a new receiving tube and was left to evaporate at atmospheric pressure and 22° C. in a microcentrifuge tube in a laminar flow chemical hood. Crystalline material was obtained and shown to be 6-azido-6-deoxy-D-glucono-1,5-lactone by 1D ¹H NMR data analysis (D₂O).

### Example 3

### Synthesis of 4-Azido-4-Deoxy-D-Glucono-1,5-Lactone

A 1 mg/mL Shvo catalyst solution was prepared by dissolving Shvo catalyst in cyclohexanone and gently vortexing for 1 minute, leaving Shvo catalyst to dissolve for 10 minutes at room temperature, again vortexing for 1 minute and then pelleting all Shvo catalyst that did not go into solution by centrifuging at 20.000 g in a microcentrifuge for 3 minutes. The supernatant was carefully aspirated and used in the following experiment.

25 mg of 4-azido-4-deoxy-D-glucose (C₆H₁₁N₃O₅, CAS 74593-35-4) was transferred into an oven-dried culture glass tube (volume ˜10 mL) with PTFE-lined cap using a metal spatula. Approximately 1.5 mL of a ˜1 mg/mL Shvo catalyst solution were added to the reaction vial and the reaction was sparged with N₂ to remove oxygen. At first the sugar did not go into solution, however after capping the glass tube and incubating at 45° C. for ˜1 h with intermittent vortexing, all particulate had gone into solution. The mixture was incubated overnight (16 h total) at 45° C.

After retrieving the reactions from the heat block the next day, the 4-azido-4-deoxy-glucose reaction showed a yellow orange tint, whereas a control reaction containing Shvo catalyst in solution but containing no sugar showed a dark reddish brown color. No visible residue/precipitate was observed in the 4-azido-glucose vial.

The sugar-containing reaction was transferred to a 1.5 mL standard microcentrifuge tube spun at 20000 g for 10 minutes at room temperature to pellet any insoluble material.

The clear supernatant was split across four 1.5 mL microcentrifuges at approximately 330 μL each—3.5 volumes of hexane were added on top of each aliquot, which at first caused a white precipitate to occur. The mixture was vortexed for 20 seconds and was allowed to stand at room temperature for 10-15 minutes, followed by centrifugation at 20000 g, at room temperature. This resulted in a syrupy, orange brown oil/syrup that pelleted at the bottom of the tube.

The supernatant was transferred to a 8 mL culture tube glass and an additional 0.6 volumes of hexane were added and the volumes were mixed by inversion. The mixture was spun for 20 minutes, 3000 g; the supernatant was removed, and a clear, syrupy material was observed on the wall of the culture tube. This material was washed down with 1 mL of acetone and evaporated with a gentle N₂ gas stream at normal pressure to give a transparent, colorless syrup.

This syrup was shown to be 4-azido-4-deoxy-D-glucono-1,5-lactone by a susceptible N-terminal protein acylation test, which showed the theoretical mass increase upon addition of the 1,5-lactone by MALDI-TOF MS. A mock reaction with just the starting material sugar did not result in any mass shift (see Table 1).

TABLE 1
N-terminal acylation test

| | Mass major peak | Delta (m/z) | Theoretical |
|---|---|---|---|
| Sample | (monoisotopic) | observed | Delta (Da) |
| Peptide alone | 3079 | N/A | N/A |
| Peptide incubated with 4- | 3079 | N/A | N/A |
| azido-4-deoxy-D-glucose | | | |
| Peptide incubated with | 3282 | 203 | 203.05 |
| substance obtained from | | | |
| Example 3 | | | |

### Example 4

### Synthesis of 3-Azido-3-Deoxy-D-Glucono-1,5-Lactone

3-Azido-3-deoxy-1,2:5,6-di-O-isopropylidene-a-D-glucofuranose (CAS 13964-23-3, Cat. No. MA06630, Carbosynth UK) was deprotected by acidic hydrolysis. 100 mg of material were dissolved in 70% acetonitrile, 20% H₂O and 10% trifluoracetic acid (v/v). The mixture was heated to 60° C. over several hours to yield the 3-azido-3-deoxy-D-glucose as a syrup. Some small amounts of remaining starting material, and only partially cleaved substance was observed by TLC analysis. This did not appear to be problematic for the reaction, however purer preparations of 3-azido-3-deoxy-D-glucose may be used as well.

The mixture was frozen at −80° C. and lyophilized overnight at low vacuum. The next morning a syrupy material was observed. The mixture was once more dissolved into a small amount of acetonitrile and water as before but not acid was added, frozen and lyophilized once more to remove any remaining traces of trifluoracetic acid. Syrupy material was once more obtained. 25 mg of this material was transferred into an oven-dried culture glass tube (volume ˜10 mL) with PTFE-lined cap using a glass Pasteur pipette.

A 1 mg/mL Shvo catalyst solution was prepared by dissolving Shvo catalyst in cyclohexanone in a microcentrifuge tube and gently vortexing for 1 minutes, leaving Shvo catalyst to dissolve for 10 minutes at room temperature, again vortexing for 1 minute and then pelleting all Shvo catalyst that did not go into solution by centrifuging at 20.000 g in a microcentrifuge for 3 minutes. The supernatant was carefully aspirated and transferred to the culture tube glass containing the azide-sugar. Approximately 1.5 mL of a ˜1 mg/mL Shvo catalyst solution were added to the reaction vial and the reaction was sparged with N₂ to remove oxygen. The mixture was incubated overnight (16 h total) at 45° C.

After retrieving the reactions from the heat block the next day, the 3-azido-3-deoxy-glucose reaction showed a yellow orange tint, whereas a control reaction containing Shvo catalyst in solution but containing no sugar showed a dark reddish-brown color. No visible residue/precipitate was observed in the 3-azido-3-deoxy-glucose vial.

The reaction was transferred to a 1.5 mL standard microcentrifuge tube spun at 20000 g for 10 minutes at room temperature to pellet any insoluble material.

The clear supernatant was split across four 1.5 mL microcentrifuges at approximately 330 μL each −3.5 volumes of hexane were added on top of each aliquot, which at first caused a white precipitate to occur. The mixture was vortexed for 20-30 seconds and was allowed to stand at room temperature for 10-15 minutes, followed by centrifugation at 20000 g, at room temperature. This resulted in a syrupy, orange brown oil/syrup that pelleted at the bottom of the tube. The supernatant was transferred to a 6-8 mL culture tube glass and an additional 0.6 volumes of hexane were added and the volumes were mixed by inversion. The mixture was spun for 20 minutes, 3000 g; the supernatant was removed, and a clear, syrupy material was observed on the wall of the culture tube. This material was washed down with 1 mL of acetone and evaporated with a gentle N₂ gas stream at normal pressure to give a transparent, colorless syrup.

Both the first obtained syrupy oil, and the secondly obtained residue on the glass wall were shown to contain 3-azido-3-deoxy-D-glucono-1,5-lactone by a susceptible N-terminal protein acylation test as in Example 3. The test showed the theoretical mass increase (203.5) upon addition of the 1,5-lactone by MALDI-TOF MS.

### Example 5

### Protein Acylation with 6-Azido-6-Deoxy-D-Glucose (6-AGDL)

### Protein Acylation

In order to test the feasibility of modifying the protein with 6-azido-6-deoxy-D-gluconolactone, 20 μM GSS-H₆-AffiEGFR was reacted with 100 mM 6-azido-6-deoxy-D-gluconolactone in 0.5 M HEPES buffer for 1 h at room temperature.

MALDI-TOF MS

20 μM GSS-H₆-AffiEGFR was reacted with 100 mM 6-AGDL in 0.5 M HEPES buffer (NaOH), pH 7.5 (total volume 50 μL), RT. After 1 h 4 μL of concentrated acetic acid were added and 10 μl reaction mixture aliquot was desalted and prepared for MALDI analysis using 0.1% TFA and final elution into 10 μL of 70% ACN/0.1% TFA C18 ZipTip method. 0.5 μL eluate were mixed 1:1 with 4-chloro-α-cyanocinnamic acid (ClCCA, CAS 69727-07-7) matrix [4 mg/mL in 90% acetonitrile (ACN), 0.1% trifluoroacetic acid (TFA) (v/v)] and spotted onto a MALDI steel target prior to detection in linear positive mode MALDI-TOF MS. The extent of acylation was calculated for species which were not acetylated within the host expression cell.

MALDI TOF-TOF MS/MS

MALDI-TOF MS/MS studies were performed using argon as a collision gas at 2 kV collision energy.

LC-MS QTOF

20 μM GSS-H₆-AffiEGFR was reacted with 100 mM 6-AGDL in the presence of 0.5 M HEPES (NaOH), pH 7.5, at RT (total reaction volume 102.5 μL). After 1 h the reaction was mixed with 3 μL of concentrated acetic acid. A 10 μL aliquot of the TFA-treated reaction mixture was desalted over a C18 ZipTip by repeatedly washing with 0.1% TFA (v/v), followed by elution with 10 μL of 70% ACN (v/v), 0.1% TFA (v/v). 8 μL of the eluent was then diluted with 0.1% (v/v) TFA to a final volume of 16 μL. 10 μL of this mixture were subjected to UHPLC-MS on an Acquity UPLC (Waters) coupled to a Xevo G2 QTOF MS (Waters) on a C18 Reversed Phase CSH column (1.0×50 mm) (Waters). The following solvent system was used at a flow rate of 0.35 mL min-1: solvent A, water containing 0.1% formic acid (v/v); solvent B, acetonitrile containing 0.1% formic acid (v/v). The column was eluted using a linear gradient from 0 to 100% of solvent B over a period of 40 minutes. The extent of acylation was estimated as the ratio of the IC peak values for acylated protein divided by the sum of all IC value for both acylated and non-acylated protein, that could participate in the reaction from deconvoluted MS data.

### Results

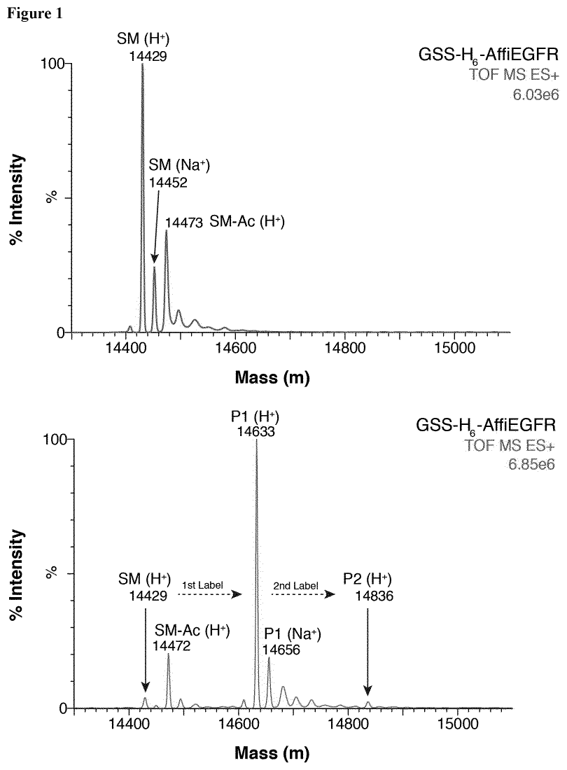

Intact acylated and non-acylated protein was chromatographically indistinguishable by LC-MS on a C18 reverse phase column. Integration of the peak areas resulted in experimental spectrums showing multiple charged species, which upon manual deconvolution exhibited mass values that were in excellent agreement with theoretical intact mass adducts. Near complete mono-functionalization was observed as indicated by the shift from the non-acylated species at m 14429 (theoretical m 14429) to the mono-acylated species at m 14633 values (theoretical m 14632) (FIG. 1). Double acylated product was observed as indicated by the small but detectable appearance of a peak at m 14836 (theoretical m 14835) (FIG. 1).

Non-reacting species at m 14472, accounting for ˜20% percent of total GSS-H₆-AffiEGFR protein (FIG. 1) were observed. It was confirmed that this peak corresponds to an expression host-derived, N-terminally mono-acetylated species (theoretical m 14471) by mass spectrometric sequencing of the corresponding tryptic peptide. Consequently, due to the blocked N-terminus of this species, little to no reaction with 6-AGDL took place.

In MALDI-TOF MS spectra intact GSS-H₆-AffiEGFR in multiply charged states with m/z 14506 (z+1), 7242 (z+2), and 4824 (z+3) were observed. The doubly charged species was the most abundant species. After reacting with 6-AGDL, almost all ion current for the non-acylated protein (m/z 7242, z+2) disappeared and a new peak at m/z 7345 (z+2) was observed in the spectrum. The mass difference between the two peaks is 206 u, which is in good agreement with the theoretically expected +203 addition for acylating wit 6-AGDL. The minor deviation may be due to the limitations of MALDI-TOF MS in linear mode in the interrogated m/z range. Furthermore, a small peak at m/z 7445 is observed, which probably corresponds to doubly acylated protein species. The remaining peak at m/z 7264 species represents the N-terminally acetylated variant, which was therefore unaffected by 6-AGDL acylation.

The extent of acylation for both QTOF MS and MALDI-TOF MS experiments was calculated from Total Ion Current (TIC) ratios of free-to-react species.

The location and extent of off-site acylation by mass spectrometric analysis of tryptic digests was tested and it was found that off-site acylation occurred as a function of pH. Off-site acylation decreased with lowering the pH to 5.5, but so did N-terminal specific acylation with lower pH (˜25%). At pH 7.3-7.5 87% N-terminal specific-acylation and ˜0.8% off-site acylation was observed. Similar N-terminal-selective acylation for pH 8.2 was observed, but off-site acylation was increased to ˜2.5%.

### Example 6

### Conjugation of Click-Chemistry Reagents onto 6-AGDL:Substrates Via Click Chemistry

### Preparation of 6-AGDL Acylated GSS-H₆-AffiEGFR

A fresh 360 mM stock of 6-AGDL was prepared by dissolving 5.18 mg of 6-AGDL in 70.8 μL of H2O. GSS-H₆-AffiEGFR was reacted at 20 μM with 100 mM of 6-AGDL in the presence of 0.5 M HEPES (NaOH), pH 7.5, RT (˜22° C.) for 1 h. The reaction was stopped by addition of acetic acid to a final concentration of 0.8 M. After acidification the reaction was transferred to a 10 kDa MWCO regenerated cellulose spin filter device (Amicon), and then washed 5 times with a 9-fold excess of 0.25 M HEPES (NaOH) pH 7.5, to remove any remaining free 6-AGDL.

### BCN Conjugation to 6-AGDL:GSS-H₆-AffiEGFR

3 mg N-[(1R,8S,9s)-Bicyclo[6.1.0]non-4-yn-9-ylmethyloxycarbonyl]-1,8-diamino-3,6-dioxaoctane (BCN-amine) (Sigma, now Merck KGaA, Cat. No. 745073, CAS No. 1263166-93-3) was dissolved in 0.5 mL of H2O, resulting in a saturated stock solution of ˜4.6 mM (˜1.5 mg/mL).

6-AGDL acylated GSS-H₆-AffiEGFR was reacted at 20 μM with 2× molar excess (40 μM) of BCN in the presence of 0.25 M HEPES (NaOH), pH 7.5 at RT (22° C.) with all the reaction components equilibrated at room temperature before proceeding (total reaction volume 50 μL). After 3 h, remaining free 6-AGDL and other buffering components were removed using a 10 kDa MWCO regenerated cellulose spin filter device b washing 5 times with a 9-fold excess of 100 mM potassium phosphate buffer (pH 7.5). A 10 μL aliquot was desalted using a C18 ZipTip, eluting in 10 μL of 70% (v/v) ACN, 0.1% (v/v) TFA. Eluate was mixed 1:1 with ClCCA matrix mixture and spotted onto a MALDI steel target prior to detection in linear positive mode in a MALDI-TOF MS. The extent of conjugation was estimated by calculating the ratio between the peak TIC of the conjugated (m₁/z) species divided by the sum of the TIC values of both conjugated and unconjugated (m₂/z) species at (z+2).

[(TIC_{(m1/z)})/(TIC_{(m1/z)}+TIC_{(m2/z)})]*100%.

### Results

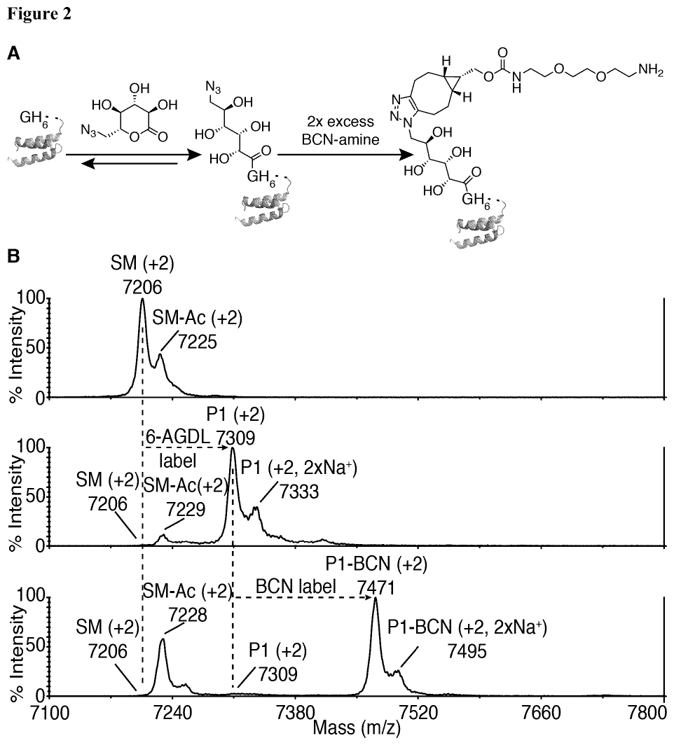

6-AGDL acylated GSS-H₆-AffiEGFR was produced as before and excess 6-AGDL was removed via spin-filtration in native conditions. Near complete (˜99%) conversion based on the TICs of m/z 7206 (z+2) (theoretical m/z 7216, native) and m/z 7309 (theoretical m/z 7317, +6-AGDL) was observed for acylation with 6-AGDL (FIG. 2). Subsequent click chemistry-mediated conjugation of the mono-functionalized, buffer-exchanged 6-AGDL:GSS-H₆-AffiEGFR with BCN-amine also resulted in almost complete conversion (˜97%) based on TICs of m/z 7309 (z+2) (theoretical m/z 7317, native+6-AGDL) and m/z 7471 (theoretical m/z 7479, +6-AGDL+BCN-amine).

### Example 7

### Acylating Non-His Tag Comprising Peptides

### Method

100 μM peptide [GGKWSKR-Beltide1 (PP7), GASGSKG-Beltide-1 (PP8), GG-Beltide-1 (PP9) or GGTYSDH-Beltide1 (PP10) was acylated with 200 mM 6-AGDL in 1 M HEPES (NaOH), pH 7.5 on ice in a reaction volume of 25 μL (adding pre-cooled 95 μL of 1M HEPES buffer into single 4 mg-containing 6-AGDL vial, dissolving it within few seconds and quickly transferring 23.75 μL into four vials each containing 1.25 μL of 2 mM PP7, PP8, PP9 or PP10 solution). After 3 h, 5 μL of the reaction mixture from each vial was taken for analysis and for the remaining volume an additional 200 mM 6-AGDL was added (each reaction mixture was transferred into a new Eppendorf tube with dry 0.8 mg 6-AGDL). The reaction mixture was kept for another 3 h and subsequently overnight at the same temperature with 5 μL from each reaction mixture is taken for the analysis at each time point.

### Results

Surprisingly, Gly-terminated peptides without oligo-His residues showed significant acylation with 50 mM lactone. For example, ˜53% acylation for GGKWSKR-Beltide-1, ˜46% for GASGSKG-Beltide-1, ˜34-36% acylation for GGTYSCH-Beltide-1 and its derivative GGTYSDH-Beltide-1. The more susceptible peptides all carried an N-terminal Gly followed by a small residue, like Gly or Ala. In comparison a single Gly-residue followed by Arg showed only ˜14% acylation (GRGDSPC). Surprisingly, introducing a di-Gly motif at the N-terminus of Beltide-1 alone boosted the conversion from ˜15% for Beltide-1 to ˜40% for GG-Beltide-1.

Contrary to previous reports, substantial (≥40%) N-terminal gluconoylation—and according to this disclosure more specifically azido-gluconoylation—can be achieved without the presence of His-residues by introducing a second N-terminal Gly residue (NH₂-Gly-Gly), conducting the acylation reaction at lower temperature and increasing acylating reagent concentration.

Similarly, 46% acylation was observed for GASGSKG-Beltide-1. Thus, it appears that an N-terminal glycine followed by a small amino acid residue like a glycine, serine or alanine may be susceptible to gluconoylation.

This was also confirmed using 6-AGDL. Single acylation of GGKWSKR-Beltide-1 peptide with 200 mM 6-AGDL resulted in ˜83% acylation. After acylating the peptide once more ˜94% singly acylated product was obtained. When leaving reaction mixture overnight ˜95% singly acylated and ˜2% double acylated product could be obtained based on Ion Count (IC) ratios of MALDI-TOF MS data.

Single acylation of GASGSKG-Beltide-1 with 200 mM 6-AGDL yielded ˜72% singly acylated product. After second acylation ˜88% singly acylated product was obtained. When left overnight ˜90% of singly acylated and ˜1% doubly acylated products were observed. ˜GG-Beltide-1 yielded ˜72% and ˜84% singly acetylated product after first and second acetylation with 200 mM 6-AGDL, respectively. Incubation of the reaction mixture overnight resulted in ˜90% singly acetylated and ˜0.5% doubly acetylated products.

Single acylation of GGTYSDH-Beltide1 with 200 mM 6-AGDL yielded ˜85% singly acylated. Reacting the singly-treated peptide once more with 200 mM 6-AGDL for 3 h resulted in ˜92% singly acylated product. Leaving the reaction mixture overnight resulted in ˜96% singly acetylated and ˜0.5% doubly acetylated products.

### Example 8

### Literature Review of Gluconoylated His-Tagged Protein

A literature review uncovered that the following tags may be susceptible to gluconoylation: H₆, A-H₆, G-H₁₀, RGS-H₆, G-H₆, G-H₈, SYY-H₆, and GGS-H₆. Thus, these tags may also be suitable for acylating with 6-AGDL.

### Example 9

### 6-AGDL:GSS-H₆-AffiEGFR Reversibility as a Function of pH

### Method

75 μM GSS-H₆-AffiEGFR was reacted with 100 mM 6-AGDL in 500 mM HEPES (NaOH), pH 7.5 (NaOH) at room temperature (23.8° C.) for 1 h by adding buffered protein solution (72 μL) to dry 6-AGDL powder (1.57 mg). The reaction was stopped by addition of acetic acid to 0.8 M. The reaction was split over three 3 kDa MWCO spin filter devices, which were washed 5 times with a 9-fold excess of the respective storage buffer to remove any remaining free 6-AGDL and other buffering components. The final volume of each retentate was adjusted to ˜90 μL to give ˜20 μM solutions with different pH values. Buffer solutions employed were: pH 4.5 [100 mM acetic acid (NH₃)], pH 7.5 [50 mM HEPES (NaOH)], and pH 8.8 [100 mM NH₃ (acetic acid)]. The reactions were stored at room temperature and 10 μL aliquots were taken at each time and desalted using ZipTip C18 resin with elution in 10 μL of 70% (v/v) ACN, 0.1% (v/v) TFA. Eluate was mixed 1:1 with ClCCA matrix mixture and spotted onto a MALDI steel target prior to detection in linear positive mode in a MALDI-TOF MS and acylation extent was determined by calculating the ratio of acylated (m_{1/z}) and non-acylated (m_{2/z}) protein at the secondary charge state with the following formula ([m_{1/z}/(m_{1/z}+m_{2/z})]*100%, z+2).

### Results

The extent of acylation reversed over time for all conditions tested. More basic solutions exhibited faster rates of hydrolysis. Approximately 30% acylation remained at pH 8.8 after storage for ˜1 week, whereas the extent of acylation at pH 7.5 and pH 4.5 remained slightly higher, with ˜60% and ˜70% respectively.

### Example 10

### Reversibility of G-H₆ and Non-His-Tagged Peptides

### Methods

100 μM of G-H₆-Beltide-1, GGKWSKR-Beltide-1 and GGTYSDH-Beltide-1 peptides were reacted at 4° C. with 6-AGDL in 1M HEPES, pH 7.5 by sequentially adding 100 mM 6-AGDL twice in 3 h intervals. Extra 6-AGDL was not removed, and the samples were stored at room temperature throughout the experiment.

2 μL from each sample were taken at each time point except for day 14. 10 μL of 2% acetic acid was added to each timepoint sample and the mixtures were desalted by C18 ZipTip chromatography. Eluate was analyzed by MALDI-TOF MS as before.

At day 14 the remaining samples underwent recovery procedure where all of the reaction mixtures were acidified by adding 20% of reaction volume of 50% (v/v) acetic acid [final concentration 7.57% acetic acid (v/v)] and then adding 10% reaction volume of 100% acetonitrile [final concentration ACN 16.6% (v/v)]. Of this mixture, 15 μL aliquots were taken, cleaned by ZipTip C18 Chromatography and analyzed by MALDI-TOF-MS instrumentation.

### Results

6-AGDL acylated G-H₆-Beltide-1 peptide showed reversibility down to ˜27% remaining acylation after storage for 14 days at room temperature (started with 94%). In contrast, non-His-Tagged Beltide-1 containing peptides GGKWSKR and GGTYSDH showed ˜64% and ˜75% remaining acylation respectively. GGKWSKR-Beltide-1 was 85% acylated on day 0 and GGTYSDH-Beltide-1 was 96% acylated on day 0.

### Example 11

### Effects of Metal Ions on Reversibility of the Reaction

### Nickel

GSS-H₆-Beltide-1 peptide was GDL-acylated and extra GDL was removed via C18 SepPack chromatography. GDL-acylated peptide was dissolved in 100 mM HEPES (NaOH), pH 7.5, 10% ACN (v/v) to a final concentration of ˜100 μM and aliquoted into 0.5 mL LoBind (Eppendorf) microcentrifuge tubes. NiSO₄ was supplemented to 0, 0.01, 0.1, and 1 mM final concentration from a 100 mM NiSO₄ stock in H₂O. A control sample was supplemented with a pre-mixed solution of NiSO₄ and EDTA, to give a final concentration of 1 mM NiSO₄ and 5 mM EDTA [stock solution 10 mM NiSO₄ and 50 mM EDTA (NaOH neutralized)]. The samples (50 μL volume each) were kept in a water bath at 37° C. Aliquots of 2 μL were taken on day 1, 3, 5, and 7, mixed with 10 μL of 5% acetic acid, purified by C18 ZipTip chromatography and analyzed by MALDI-TOF-MS instrumentation.

Supplementation with low concentrations of NiSO₄ seemingly abolished reversibility all together for the period tested.

### Zinc

GSS-H₆-Beltide-1 peptide was GDL-acylated and extra GDL was removed via C18 SepPack chromatography. GDL:peptide was dissolved in 100 mM HEPES (NaOH), pH 7.5 with or without ZnSO₄ (0.2 mM and 2 mM) to a final concentration of ˜100 μM. ACN was added to 10% v/v to reduce possible nanodisc formation and precipitation of the peptide to the plastic microcentrifuge wall. The reaction mixtures (100 μL volume each) were kept in a water bath at 37° C. Aliquots of 2 μL were taken at day 3, mixed with 10 μL of 5% acetic acid, purified by C18 ZipTip chromatography and analyzed by MALDI-TOF-MS instrumentation. The sample on day 7 underwent a ‘peptide recovery’ procedure, as limited peptide was observed in solution. The remaining reaction mixture (80 μL) was acidified by adding 50% (v/v) acetic acid. The amount of acetic acid to be added was calculated as 20% of the remaining reaction volume [final concentration 7.57% acetic acid v/v]. Likewise, 10% reaction volume of 100% acetonitrile were added (final concentration acetonitrile ˜16.6% v/v) and the reaction was mixed by quick vortexing. Of this mixture, 15 μL aliquots were processed via C18 ZipTip chromatography and analyzed by MALDI-TOF-MS instrumentation. We speculate that the peptide had bound to the microcentrifuge wall and assumed that the precipitation affected both acylated and non-acylated species equally.

Near quantitative acylation was observed at the beginning of the experiment. A marked difference in the extent of acylation for sample stored in ZnSO₄ containing buffers could already be discerned after 3 days in comparison to the control. On day 7 of incubation, the control sample without ZnSO₄ showed extensive reversibility, with only ˜7% acylated peptide remaining. In contrast, samples that were stored under identical conditions but in the presence of 0.2 mM, or 2 mM ZnSO₄ showed ˜80% and ˜75% remaining gluconoylation.

### Copper

1 mM G-H₆-Beltide-1 (˜1 mg of peptide) was acylated with 100 mM GDL in 200 mM HEPES (NaOH) pH 7.5 for 1 h at room temperature in a total volume of ˜300 μL. Upon addition of buffer to peptide but before addition of GDL, precipitation was clearly observed. This precipitation may be due to the theoretical pI of G-H₆-Beltide-1 being close to pH 7.5, or because G-H₆-Beltide-1 is a Beltide-1 variant. Beltide-1 is known to form nanodiscs in solution upon contact with membrane proteins or lipid components (Midtgaard et al. 2014; Martos-Maldonado et al. 2018). GDL was added to 100 mM from a freshly prepared 1 M stock solution. The solution remained cloudy. Upon addition of ACN to 10% v/v the solution cleared up immediately, indicating the dispersal of any precipitate or possible nanodiscs. After 1 h of addition of GDL, the reaction was acidified with 100 μL of 50% acetic acid (˜8.7 M) to give a final concentration of >2 M acetic acid in solution, overwriting any buffering capacity imposed by HEPES buffer, gluconic acid or remaining GDL. Unreacted and hydrolyzed GDL was removed by binding the acidified peptide to an equilibrated Sep-Pak C18 cartridge and washing with 6 mL aqueous 0.1% TFA (v/v). The peptide was eluted with 5 mL of 70% ACN (v/v) acidified with 0.1 TFA (v/v) and aliquoted over fourteen 1.5 mL LoBind Eppendorf tubes followed by freeze-drying. Upon reconstitution with 350 μL each vial was assumed to contain a solution with ˜100 μM concentration. The acylated peptide was analyzed by MALDI-MS and initial acylation extent was estimated to be ˜96%, stored at −20° C. until further usage.

To reconstitute peptide, aqueous 20% ACN (v/v) was added with 2× buffer strength (100 mM) to give a peptide reaction master stock at 2× concentration (peptide at 40 μM). The resuspension was mixed by vigorous vortexing and aliquoting into standard 0.5 mL reaction vials (CuSO₄ trials were performed in Heathrow Scientific HD4422 microcentrifuge tubes) that contained pre-prepared buffer conditions. For a typical reaction setup, the order of addition was: water, additives such as CuSO₄ and finally addition of 2× peptide reaction master stock at 2× concentration to yield 1× with a final concentration of ˜10% ACN (v/v), 50 mM buffer, various concentrations of additives and 20 μM peptide. Samples were mixed by gentle flicking (2-3 times) and shake down by hand. Samples were incubated in a water bath at 37° C. 10 μL samples were withdrawn for each timepoint and acidified with 5 μL of 20% acetic acid followed by Zip Tip clean-up and spotting in Cl-CCA matrix on MADLI-TOF MS steel target.

The results are shown in FIG. 3. Samples mixed with CuSO₄ showed a decreasing rate of reversibility as a function of increasing CuSO₄ concentration.

### Example 12

### Effect of Boric Acids on Reversibility of 6-AGDL Acylated GSS-H₆-Beltide-1

### Method

pH 4 buffer was prepared by combining 945 μL of acetic acid (100%) with ˜10 mL of H2O, followed by titration to pH 4.01 using ˜900 μL of 4 M NaOH. The volume was then made up to 15 mL to give a 1 M stock solution.

pH 6 buffer was made by preparing a 1 M stock solution of citric acid monohydrate and by making a solution of 1 M sodium citrate dihydrate. The two solutions were mixed to give a 1 M stock solution of pH 6.

pH 7.5 buffer was made by mixing a 1 M stock solution of potassium phosphate monobasic with 1 M solution of potassium phosphate dibasic to give a pH of 7.5.

pH 8.8 buffer was prepared by titrating 1 M solution of acetic acid with ammonium solution to give pH 8.8.

20 μM 6-AGDL modified peptide was stored in 100 μL aliquots in 200 mM buffer as indicated. The vials (triplicates) were incubated in a 37° C. water bath throughout the experiment and were retrieved for sample acquisition for <30 mins at room temperature before they were returned to the water bath.

10 μL of subsamples for pH 4, 6 and 8.8 were acidified with 5 μL of 20% (v/v) acetic acid and subjected to C18 ZipTip purification. Samples were analyzed in positive reflectron mode with MALDI-TOF MS.

pH 7.5 samples were stored in the presence of 10% ACN (v/v) to counteract precipitation. For pH 7.5 samples, 15 μL aliquots were taken for each time point and desalted using 0.1% TFA-70% ACN/0.1% TFA C18 Zip-Tip procedure. 0.5 μL of the Eluate was mixed 1:1 with C1CCA matrix mixture and spotted onto a MALDI steel target prior to detection in reflector positive mode MALDI-TOF MS.

### Results

Reversibility was effectively inhibited over a period of 7 days at 37° C. with >50 mM boric acid at pH 8.8, whereas non-boric acid treated samples quickly reversed to their non-acylated state. Supplementation with boric acid modulated the reversibility at pH 6, whereas no effect was observed in acidic condition (pH 4). Less boric acid supplementation was required to reach similar protection at neutral to slightly basic pH (≥7.5) conditions compared to more acidic conditions (pH 6). Least boric acid supplementation was required in alkaline conditions (pH 8.8) with 5 mM boric acid supplementation providing a better protective effect as compared to 100 mM boric acid at pH 6.

Surprisingly, the addition of boric acid at the concentrations tested here (50 mM) did not measurably inhibit the serine protease trypsin and allowed mapping of the acylation label to the N-terminal peptide. Without including boric acid in the sample, the extent of acylation may be underestimated. This is because trypsin digests are most preferably performed at alkaline pH (˜pH 8.4) and elevated temperature (37° C.), conditions at which trypsin shows the highest activity, but the acylation modification also reverses most rapidly. Hence boric acid protects the reversal of the acylation modification and allows to assess the true extent of labelling, whereas excluding boric acid or another suitable diol-ester forming agent would allow the modification to reverse throughout the time course of the enzymatic digestion.

Noteworthy, boric acid also protected endogenously derived non-azido gluconoyl modification (from E. coli BL21 expression) and addition of boric acid may be used to allow more accurate quantification of the extent and mapping of the position of any in vivo derived gluconoylation. Characterizing the extent of in vivo gluconoylation and phospho-gluconoylation is often performed analysis for therapeutic recombinant proteins and is in some cases required by the FDA for novel drug applications and/or biosimilar approval. However, up until now these analyses have been performed without diol-ester forming agents which protect the modification, thus the extent of the modification may be underestimated in the currently employed assays.

### Example 13

### Benchmarking Achievable Extent of Acylation and Selectivity for 6-AGDL Against 4-Methoxyphenyl 2-Azidoacetate

### Method

4-methoxyphenyl 2-azidoacetate (4MPAA) stock was prepared by diluting light yellow, oily substance (10.14 mg) to a final volume of 49 μL with ACN to give a 1 M stock solution. This stock solution was diluted 1:40 with ACN [195 μL ACN to 5 μL of 1M stock 4MPAA] to give 25 mM reaction stock and was used to supply 4MPAA to the reactions.

6-AGDL was prepared by dissolving 3.21 mg of white powder in 16 μL H2O to give a 1 M stock 6-AGDL solution. This solution was used to supply to the reaction.

Peptides were reacted at 1 mM in a volume of 25 μL in 200 mM HEPES buffer at pH 7.5, 10% (v/v) ACN (final concentration) at 4° C. or 22° C. (RT) for the times indicated. ACN was supplied to 4MPAA samples as the solvent for the reagent and the ACN concentration in the final sample was also 10% (v/v). 2.5 μL ACN was supplied to 6-AGDL samples to help dissolve G-H₆-Beltide-1 which was visibly precipitating [final concentration ACN 10% (v/v)].

5 μL reaction mixture were withdrawn after 1, 18 and 24 h, mixed with 45 μL H2O to give a 100 μM peptide solution, which was acidified with 25 μL 20% (v/v) acetic acid [final concentration of ˜1.18 M acetic acid]. 10 μL of this preparation was used a substrate for standard C18 Zip Tip purification.

### Results

Both base peptides, G-H₆-Beltide-1 (PP1), and GSS-H₆-Beltide-1 (PP6) were modified using 4MPAA or 6-AGDL at all temperatures tested.

100 mM 6-AGDL treated peptide PP1 was quickly mono-functionalized (>98%) within 1 hour upon addition of 6-AGDL at both 5.4° C. and RT (23° C.) with very little off-site acylation (˜0.3%). In contrast, acylation with 2.5 mM 4MPAA showed only ˜10% (5.4° C.) or ˜19% (RT) of mono-functionalized peptide (Table 2).

The same extent of acylation as previously reported for acylation of 1 mM PP1 (G-H₆-Beltide-1) with 2.5 mM 4MPAA at 4° C. over a period of 24 h [previously reported 92% mono-, 8% di-functionalized protein (Martos-Maldonado et al. 2018)] was not observed. When PP1 was reacted with 2.5 mM 4MPAA for 24 h at 5.4° C., 60% mono-functionalization with ˜5% dual-functionalization was obtained. It was speculated that the preparation of 4MPAA used may not have been as pure as the reported one. Upon increasing the concentration of 4MPAA to 4 mM, ˜67% mono-functionalized peptide could be obtained for PP1, albeit at the cost of increasing offsite dual-functionalization (˜7-8%) (24 h, 5.4° C.).

Compared to PP1, PP6 was less reactive with any acylation reagent tested after reacting for 1 h. Nevertheless mono-functionalized peptide was observed at over >80% at either temperature after 1 h incubation with 100 mM 6-AGDL. Importantly, mono-functional acylation to ≥98% with little dual-functionalization (˜0.8%) could still be obtained, when incubating with 100 mM 6-AGDL at 5.4° C. for prolonged periods (18 or 24 h). In contrast, acylation of PP6 with either 2.5 mM or 4 mM 4MPAA for the same amount of time resulted in only ˜40%, and ˜53% when incubated at 5.4° C. Strikingly, significant dual-functionalization was observed as a function of increasing 4MPAA concentrations (˜5%, and ˜10%).

TABLE 2
Selectivity Comparison of 6-AGDL Against 4-MPAA

| | | | 2.5 mM 4-methoxyphenyl 2-azidoacetate | | | 100 mM 6-AGDL | | | 4 mM 4-methoxyphenyl 2-azidoacetate | | | |
|---|---|---|---|---|---|---|---|---|---|---|---|---|
| | | | Single | Double | Non- | Single | Double | Non- | Single | Double | Triple | Non- |
| PP1 | Time | Temp. | acylation | acylation | acylated | acylation | acylation | acylated | acylation | acylation | acylation | acylated |
| | 1 h | 5.4 | 10 ± 2.7% | 0.4 ± 0.1% | 90 ± 2.7% | 98 ± 1.0% | 0.3 ± 0.1% | 1.9 ± 0.9% | | | | |
| | 18 h | 5.4 | 59 ± 1.4% | 4.6 ± 0.4% | 36 ± 1.5% | 98 ± 0.5% | 0.7 ± 0.0% | 1.4 ± 0.5% | | | | |
| | 24 h | 5.4 | 60 ± 1.5% | 5.1 ± 0.5% | 34 ± 1.9% | 96 ± 0.6% | 0.9 ± 0.1% | 2.8 ± 0.6% | 67 ± 4.3% | 7.7 ± 0.7% | 0.5 ± 0.1% | 25 ± 4.5% |
| | 1 h | 23 | 19 ± 2.9% | 0.5 ± 0.1% | 80 ± 2.9% | 98 ± 0.4% | 0.4 ± 0.1% | 1.2 ± 0.3% | | | | |
| | 18 h | 23 | 47 ± 3.9% | 3.0 ± 0.4% | 50 ± 4.0% | 88 ± 2.8% | 0.5 ± 0.1% | 12 ± 2.8% | | | | |
| | 24 h | 23 | 51 ± 3.6% | 3.4 ± 0.5% | 46 ± 3.6% | 82 ± 3.5% | 0.6 ± 0.1% | 17 ± 3.6% | 48 ± 9.2% | 0.4 ± 0.4% | 0.4 ± 0.1% | 48 ± 10.0% |
| | | | Single | Double | Non- | Single | Double | Non- | Single | Double | Triple | Non- |
| PP6 | Time | Temp. | acylation | acylation | acylated | acylation | acylation | acylated | acylation | acylation | acylation | acylated |
| | 1 h | 5.4 | 8 ± 1.7% | 0.3 ± 0.1% | 92 ± 1.7% | 84 ± 1.2% | 0.3 ± 0.1% | 15 ± 1.2% | | | | |
| | 18 h | 5.4 | 38 ± 0.5% | 4.2 ± 0.8% | 58 ± 1.4% | 98 ± 0.2% | 0.8 ± 0.2% | 1.4 ± 0.1% | | | | |
| | 24 h | 5.4 | 40 ± 3.1% | 4.5 ± 0.2% | 55 ± 3.3% | 98 ± 0.7% | 1.0 ± 0.1% | 1.4 ± 0.7% | 53 ± 7.8% | 10.1 ± 3.9% | 0.7 ± 0.3% | 37 ± 11.9% |
| | 1 h | 23 | 17 ± 1.2% | 0.6 ± 0.2% | 83 ± 1.4% | 90 ± 1.9% | 0.4 ± 0.0% | 9.2 ± 1.8% | | | | |
| | 18 h | 23 | 29 ± 0.2% | 1.3 ± 0.1% | 70 ± 0.1% | 90 ± 1.5% | 0.5 ± 0.1% | 9.9 ± 1.4% | | | | |
| | 24 h | 23 | 35 ± 7.8% | 2.1 ± 1.3% | 63 ± 9.1% | 91 ± 3.9% | 0.6 ± 0.1% | 8.3 ± 4.0% | 36 ± 3.3% | 2.5 ± 0.2% | 0.2 ± 0.0% | 62 ± 3.5% |

### Example 14

### mP-AGE for Low-Cost Analysis of (Azido)-Gluconoylated Proteins

It has previously been shown that singly-gluconoylated proteins up to 16.7 kDa Mw can be resolved with methacrylamido phenylboronate acrylamide gel electrophoresis (mP-AGE), a modified SDS-PAGE matrix that relies on the dynamic equilibrium between boronic acid derivative and 1,2-, as well as 1,3-diols (Pereira Morais et al. 2010). mP-AGE gels are not yet commercially available; however, several facile synthesis routes exist for the polymerizable 3-[(Methyl-)acrylamido] phenylboronic acid derivatives [(M)PBA], of which PBA has also recently become commercially available from Sigma (now Merck) (Cat. No. 771465-1G) (Morais et al. 2012, 105; D. Li et al. 2015). Casting mP-AGE gels is as simple as adding an aqueous solution of (M)PBA to the resolving SDS-PAGE gel mixture prior to polymerization (Pereira Morais et al. 2010).

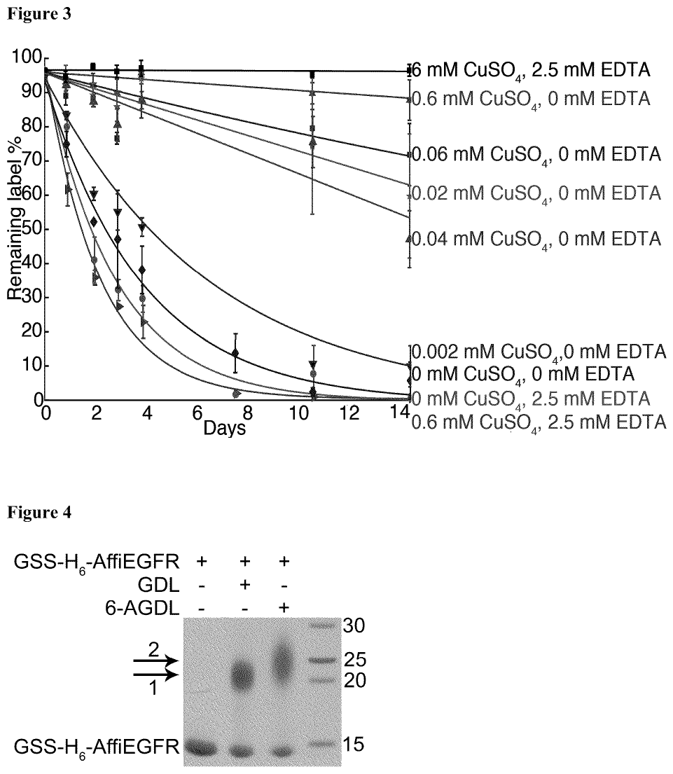

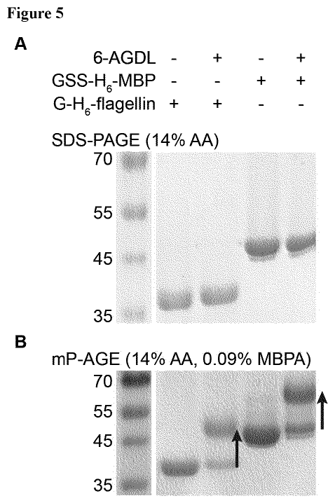

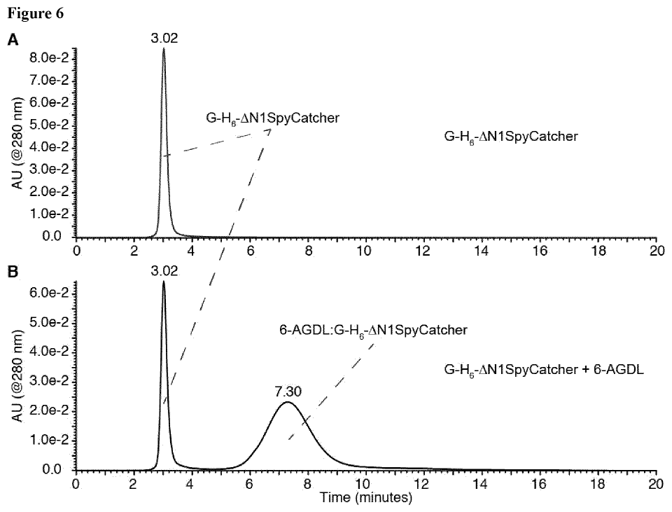

This approach was tested by acylating and resolving a small number of model proteins with mP-AGE and traditional SDS-PAGE (see FIGS. 4 and 5).

### Example 15

### Selective Enrichment of an Azido-Gluconoyl-Variant Species from a Mixture of Acylated and Non-Acylated Protein

### Sample Preparation

GSS-H₆-AffiEGFR was essentially produced as before in a gluconoylation deficient E. coli BL21(DE3) derivative strain. We acylated the protein as before with 6-azido-6-deoxy-glucono-1,5-lactone (6-AGDL), excess 6-AGDL was removed by spin-filtration, and the extent of acylation was determined to be ˜80% by MALDI TOF-MS. We spiked 1.6 μL of the acylated preparation at ˜125 μM with 500 μL of non-acylated GSS-H₆-AffiEGFR at ˜157 μM. 62.5 μL of 4 M NaCl was added (final conc. ˜330 mM) and 200 μL of 25 mM AmBic pH 8.5 was added. This translates to ˜490 excess of non-acylated compared to acylated protein. A subsample (20 μL) was taken for ZipTip C18 prior to BAC purification.

### Resin Preparation

50 μL of slurry [m-Aminophenylboronic acid-agarose bead support (Sigma)] were transferred to a spin column (0.22 μm pore size, hydrophilic PVDF membrane, 0.5 mL volume, non-sterile, Sigma Cat. No. UFC30GV00). 500 μL water were added and the column was spun for 30 seconds at 5000 RPM (˜1677 g) in an Eppendorf MiniSpin centrifuge at room temperature. The flow-through was discarded and 500 μL of 25 mM AmBic pH 8.5 were added. The mix was spun as before and the flow-through was discarded.

### Sample Binding

600 μL of the mixed sample were contacted with the equilibrated resin at 4° C. for 30 mins. The resin protein mix was mixed every 10 minutes by gentle pipetting. The column was centrifuged as before and the flow-through was collected as a fraction.

### Washing

The column was incubated with 500 μL of 500 mM NaCl, 25 mM AmBic pH 8.5. The mixture was mixed by gentle pipetting on the column, followed by centrifugation as before. The flow-through was collected as fraction “wash 1.” This procedure was repeated for a total of 5 times to give fraction “wash 2,” “wash 3,” “wash 4” and “wash 5.”

### Elution

60 μL of 200 mM ammonium acetate at pH 4.0 were added to the resin and elution was allowed to take place over 5 minutes at room temperature. The elution was collected by centrifugation into a new clean tube (fraction “Eluate target #EL”).

### Sample Processing for MALDI TOF-MS

Samples were Zip Tipped (C18) as before and spotted onto a MALDI steel target plate in MALDI matrix. Samples were shot in positive linear mode to determine the relative abundance of species.

### Results

The spectra of the non-purified, spiked mixture did not show any ion current for the azido-gluconoylated species. However after BAC enrichment, the species is clearly visible and makes up ˜68.2% [z 1; (2103.9)/(2103.9+979.2))=0.682%], ˜71.4% [z 2; (3897.3/(3897.3+1559.2))=0.714], ˜75.6% [z 3; (672.5/(672.5+216.5))=0.756] of the respective charge state species. Assuming that the initial concentration before enrichment was ˜0.2% (490-fold lower than the non-acylated species), this represents ˜350-fold enrichment of the acylated species over the non-acylated one.

The expected m/z at z 1 for the Met-cleaved protein, based on the primary amino acid sequence obtained from DNA sequencing, is 14421.0055 Da, whereas the average Mw would be expected to be 14430.0533. The observed m/z values at z 1, 2 and 3 are in good agreement with the theoretical m/z values for non-acylated protein in the top row. Mono-azido-gluconoylated species would be expected to display a m/z increase of 203.5 at z 1 and 101.75 at z 2 respectively. The observed m/z at z 2 of 7317.3 gives a delta of 102.8 m/z in combination with the m/z at z 2 of 7214.5, which is in excellent agreement with a mono azido-gluconoylated protein.

The example demonstrates that variant gluconoyl residues, such as 6-azido-6-deoxy-gluconoyl can be also selectively enriched via their diol function.

Elution methods are several-fold, either a drop in pH, or competition with a diol, e.g., glycerol, Tris, or a sugar, and sugar derivatives may be used to compete the acylated molecule off the solid support. Since the interaction is dynamic covalent, prolonged incubation with excess binding buffer also allows isocratic elution.

The outlined method has the advantage that non-acylated species do not need to be removed by other, more tedious techniques such as ion-exchange, which requires fine-tuning, whereas BAC is a general affinity method and is expected not to require extensive optimization.

### Example 16

### Selective Enrichment of an Azido-Gluconoyl-Variant Species from a Mixture of Acylated and Non-Acylated Protein Using an on-Line System

### Column Equilibration

A commercially available TOSOH TSKgel Boronate-5PW column was also tested for resolving acylated from non-acylated substrates according to the disclosure on a standard HPLC system (Cat. No. 0013066, Tosoh Biosciences GmbH, Germany; 7.5 mm internal diameter x 7.5 cm length with 100 nm (1000 Å) pore size made of polymethacrylate base material bonded with m-aminophenyl boronate). The mobile buffer was 100 mM ammonium acetate (pH 8.5) and was used at a flowrate of 0.5 mL/min throughout.

### Sample Preparation and Application

Native, as well as a mixture of native and 6-azido-6-deoxy-D-glucono-1,5-lactone treated sample (G-H₆-ΔN1SpyCatcher, purified by single step immobilized metal-affinity chromatography to >90% by SDS-PAGE stained with Coomassie Blue) were sequentially applied to the column (20 μL of a 200 μM sample in pH 4.5 ammonium acetate buffer, 10 mM). In case of the sample contacted with 6-azido-6-deoxy-D-glucono-1,5-lactone, excess acylating reagent was first removed by repeated spin filtration through a 3 kDa MWCO, prior to loading onto the diol-binding column. The analytes were continuously detected at 280 nm.

### Results

Whereas the native protein eluted in only in a single peak, the sample mixture was resolved as two species—one peak with the retention time of the unmodified material and a new, later eluting peak. Peak fractions were collected manually for each peak, acidified, desalted (ZipTip C18) and subjected to MALDI-TOF MS intact mass analysis in linear positive mode as before. The material collected from the earlier eluting peak was indistinguishable from the untreated starting material by mass, whereas the later eluting peak only showed the expected mass for a singly modified species. No unmodified starting material, nor immobilized metal-affinity chromatography associated peptide or protein contaminants were identified in the mass spectrum of the later eluting peak.

### Example 17

### Manufacture of a Vaccine for the Prevention of SARS-CoV-2

### Method

Qbeta VLPs (SEQ ID NO: 86) were produced in E. coli. 5 mg/mL Qbeta VLPs were activated with 2 mM NHS-PEG8-BCN (CAS. 1608140-48-2; SiChem Cat No. SC-8108) for 2 h at room temperature in PBS pH 7.4. Excess label reagent was removed by spin filtration into 50 mM borate pH 8.2 using 100 kDa MWCO spin filter (PES membrane).

The receptor binding domain (RBD) of SARS-CoV-2 (SEQ ID NO: 87) was recombinantly produced in HEK293 or Pichia pastoris cells, purified by Ni-NTA affinity, and size exclusion chromatography, and then stored in PBS at 1 mg/mL. The RBD was activated with 100 mM 6-AGDL in 200 mM potassium phosphate buffer (pH 7.5) for 80 minutes at room temperature prior to spin filtration into 50 mM borate pH 8.2. The final concentration of the activated RBD was 2 mg/mL and the solution was stored at 4° C. until further usage.

Activated RBD and activated Qbeta were contacted at a 4:1 molar ratio for 14 h overnight at 4° C. The conjugation reaction was analyzed by SDS-PAGE. Excess RBD was removed by gel filtration 50 mM borate pH 8.2.

The conjugate material was then used to immunize mice at 10 μg per dose formulated in AddaVax (Invivogen) according to the manufacturer on Day 0. Boosts with the same dose and formulation were performed on day 14 and day 28. Sera samples were taken before each immunization for immunological studies. Animals were sacrificed on day 42.

Endpoint ELISA were performed according to standard procedures known in the art against non-activated HEK-produced RBD.

Pseudovirion neutralization assays were performed with samples obtained at day 42 according to standard procedures known in the art.

Live virus neutralization assays were performed with serum samples from day 42. Inhibition of infection and/or replication of the SARS-CoV-2 virus (hCoV19, D614G(S)) was tested on Vero E6 cells in vitro with quantitative PCR (qPCR) as read-out.

Quick-RNA viral kit (Zymo Research) was used to extract RNA and was performed in a BSL-3 lab following manufacturer's conditions and starting from 150 μL of supernatant from infected/challenged cells at 48 h post infection (0.1 MOI). Sensifast cDNA synthesis kit (Meridian) was used following the manufacturer's instructions. Specifically: 4 μl of 5× buffer+1 μL transcriptase+12 μL RNAse free water+3 μL of RNA extracted in the previous procedure. Final volume=20 μL. Incubation times: 10 min at 25° C./15 min at 42° C./5 min at 85° C./Hold at 4° C.

CFX384 Touch Real-Time PCR Detection System, Biorad was used for quantitation: Hold stage: 50° C. 15 min+95° C. 1 min (Ramp=1.6° C./sec), PCR: 95° C.−10 sec+60° C.−1 min×40 cycles. (Ramp=1.6° C./sec) using the 2020 Center of Disease control (CDC) N₁ primers and probe set targeting the nucleocapsid “N” gene of SARS-CoV-2. FW: 5′-gaccccaaaatcagcgaaat-3′ (SEQ ID NO: 88) RV: 5′-tctggttactgccagttgaatctg-3′ (SEQ ID NO: 89) probe: 5′-FAM-accccgcattacgtttggtggacc-BHQ1-3′ (SEQ ID NO: 90). Sensifast SYBR No-ROX kit (Meridian), was used following the manufacturer's instructions. Specifically: 5 μl of Sensifat PCR mix (Taq included)+0.5 μL of each primer (18 uM stock)+0.5 μL FAIM primer (5 uM stock)+3.5 μl of the viral/sample RNA. Final volume=10 μL.

Samples were processed in triplicates. Cq values are plotted as symbols, with the arithmetic mean plotted as a line with error bars (standard deviation) shown in grey. Typically, human convalescent sera showed Cq values 22-23 in this assay and are known to be protective. Hence the cut-off was set at Cq 22 (mean) to call protective correlates for the mice sera.

### Results

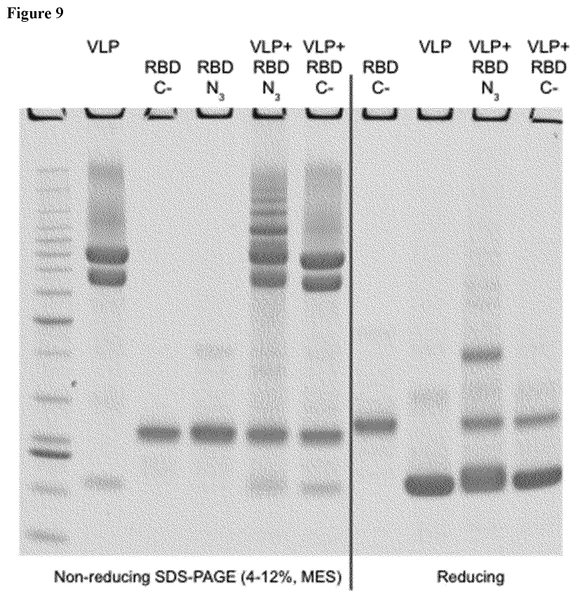

Contacting activated RBD with activated VLPs produces a conjugate band as observable by reducing SDS-PAGE, whereas reaction of activated VLPs with non-activated RBD does not show the expected adduct band (see FIG. 9). In non-reducing conditions covalent decoration of the disulphide-linked hexamer and pentamers with the RBD can be observed, showing multiple high molecular weight adduct bands.

The reduced adduct band was excised, destained, alkylated, proteolytically digested. MALDI-TOF MS/MS analysis revealed the presence of both Qbeta and RBD derived peptides, confirming the covalent conjugation of the two proteins (data not shown).

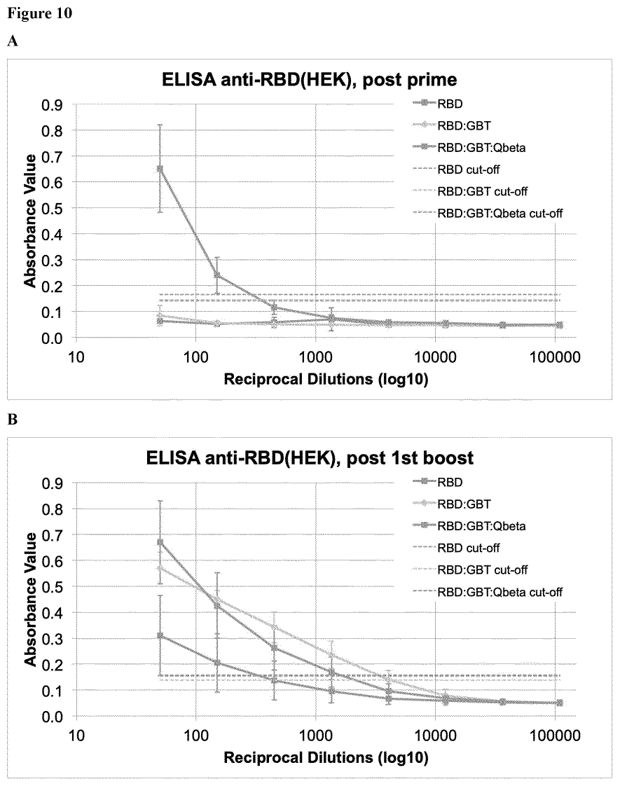

Immunization studies showed that RBD conjugated to VLPs produced a response after a single injection (post primer, see FIG. 10A). ELISA titers showed a detectable anti-RBD response in the Qbeta:RBD conjugated group only, whereas adjuvanted RBD alone, or Qbeta VLPs alone did not show a measurable response. Hence serum conversion could be achieved with a single administration of the conjugate vaccine, but not with the monomeric AddaVax adjuvanted RBD formulations.

The anti RBD titers could be boosted upon subsequent immunizations. Surprisingly, simply 6-AGDL activated RBD that was not multimerized onto VLPs also showed improved immunogenicity compared to the non-activated RBD (8 out of 8 mice seroconverted) with higher anti-RBD titers, suggesting that in this particular formulation, that the treatment with the 6-AGDL linker itself can act as an adjuvant (see FIG. 10B).

Correlation of the immune response with viral inactivation was queried in a pseudotyped virus assay. The best results were obtained in the Qbeta:RBD conjugated group at day 42 (after a total of 3 immunizations) (see Tables 3 and 4). Surprisingly, the activated RBD performed better than the non-activated RBD, even without conjugation to a VLP scaffold.

TABLE 3
Pseudovirus Neutralization Capacity

| Group | % of neutralization at dilution 1:300 |
|---|---|
| Qbeta | 17.24 |
| RBD(HEK) | 20.86 |
| RBD(HEK) 6-AGDL activated | 43.44 |
| RBD(HEK) 6-AGDL linked to Qbeta | 71.63 |
| rabbit control polyclonal Abs | 99.62 |

TABLE 4
Pseudovirus Neutralization Capacity

| Group | 50% neutralization titer (1:x) |
|---|---|
| RBD(HEK) 6-AGDL activated | 258 |
| RBD(HEK) 6-AGDL linked to Qbeta | 798 |
| rabbit control polyclonal Abs | 1326 |

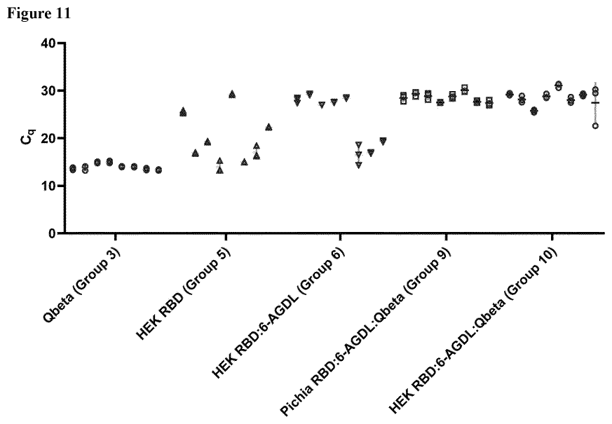

The superior performance of multimerized RBD was also observable in a live virus neutralization assay using SARS-CoV-2 as a challenge. Once more the RBD linked to Qbeta via the azide linker showed neutralizing activity (Cq>22) in vitro for all animal sera tested (n=8), whereas monomeric AddaVax adjuvanted RBD only induced protection in 3 out of 8 sera tested, even after three immunizations (see FIG. 11). Interestingly, the activated RBD also appears to perform better than just the RBD, inducing neutralizing responses in 5 out of 8 mice sera. Protective responses were also obtained using Pichia pastoris produced protein instead of HEK293 produced RBD using the same conjugation approach.

### Example 18

### Additional Boron-Containing Reagents to Modulate Reversibility

### Method

To explore the reversibility modulating effect of various boron-containing molecules, a small library of reagents was tested at pH 7.5 (50 mM HEPES/NaOH buffer) and pH 6.8 (50 mM phosphate/KOH buffer). The capacity of the reagents to inhibit the reaction was tested at 5 mM concentration on 50 μM of an N-terminally gluconoyl-labelled Beltide-1 derivative peptide PP6 (GSSHHHHHHIDWLKAFYDKVAEKLKEAF), under heat-accelerated deacylation conditions (50° C.).

Boric acid (CAS 10043-35-3, Sigma Cat. No. B0394), methylboronic acid (CAS 13061-96-6, Sigma Cat. No. 165336), phenylboronic acid (CAS 98-80-6, Sigma Cat. No. P20009), 3-Aminophenylboronic acid monohydrate (CAS 206658-89-1, Sigma Cat No 287512), 2-formylphenylboronic acid (CAS 40138-16-7, Sigma Cat. No. 431958), 4-formylphenylboronic acid (CAS 87199-17-5, Sigma Cat. No. 431966), and the FDA-approved tavaborole (AN2690, 5-Fluoro-1,3-dihydro-1-hydroxy-2,1-benzoxaborole; CAS 174671-46-6, Cayman Chemicals Cat. No. 23101) were tested.

### Results

Several boron containing molecules could be used to reduce or entirely inhibit the reversibility of the gluconoyl peptide to the unmodified peptide (i.e., without gluconoyl modification) over the period and in the buffer conditions tested. A general trend was observed that with decreasing pKa of the additive tested compounds, the conjugate band could also be protected at lower solution pH values (See Table 5).

TABLE 5
Remaining Gluconoyl-Label After Incubation at Indicated pH and Time

| | Literature | pH 6.8 after | pH 7.5 after |
|---|---|---|---|
| | pKa | 15 h at 50° C. | 15 h at 50° C. |
| boric acid | 9.15 | ~8% | ~69% |
| methylboronic acid | 10.4 | ~6% | ~59% |
| phenylboronic acid | 9.0 | ~31% | ~83% |
| 3-aminophenylboronic acid | 8.9 | ~31% | ~84% |
| 2-formylphenylboronic acid | 7.5 | ~95% | ~99% |
| 4-formylphenylboronic acid | 7.8 | ~46% | ~95% |
| tavaborole | 6.7-6.97 | ~87.9% | ~87% |
| Control (nothing added) | — | ~8% | ~67% |

Reaction supplemented with 5 mM of additive; calculated as the ratio of the TIC for the labelled divided by the sum of TICs of both labelled and unlabeled species.

The results show that by choosing a boron-containing acid or hemi-ester with suitable pKa, it is possible to protect acylated conjugate bonds at various solution pH values. However, other factors such as steric accessibility, and intramolecular coordination clearly also play a role, as observed for 2-formylphenylboronic acid and tavaborole, when compared to 4-formylphenylboronic acid

### Example 19

### Covalently Trapping a Boronic-Diol Ester of Acylated Species

Encouraged by these results, and inspired by recent reports, we wondered if the otherwise by MALDI-TOF-MS unobservable 2-formylphenylboronic acid derived ester could be trapped covalently by addition of an N-hydroxylamine species—(Brittain et al., 2016) AND (Meadows et al., 2017).

The method and composition described here differ from the prior art in several ways. Whereas Meadows synthetically synthesized a 3,4-dihydroxyphenylalanine (DOPA) containing peptide to provide a targetable diol, in this disclosure suitable diol(s) are site-specifically introduced at the N-terminus of a recombinant protein according to the disclosure (e.g., GlyHis-terminated for example). Diol introduction is achieved by treating a suitable protein according to the disclosure with glucono-1,5-lactone or any other suitable carbohydrate lactone, including handle-substituted carbohydrate lactones. Once an acylated, diol-containing peptide or protein substrate is obtained, it is treated with a 2-formylphenylboronic acid derivative and a N-hydroxylamine derivative compound.

Method for Covalently Trapping a Boron-Diol Ester of a Substrate Previously Acetylated and Treated with 2-Formylphenylborone Acid

20 μL of an aqueous solution of 200 μM N-terminally gluconoylated peptide (GSSHHHHHHGGTYSAHFGPLTWVAKPQGG) was mixed with 2.5 μL of 20 mM 2-formylphenylboronic acid. Thereafter, 2.5 μL of 20 mM N-tert-butylhydroxylamine hydrochloride was added to the reaction mixture. After 2 h of reaction at room temperature, 10 μL of reaction mixture was withdrawn, mixed with 2 μL of 50% (v/v) acetic acid, cleaned using C18 ZipTip and analyzed by MALDI-MS.

### Results for Trapping a Gluconoylated Substrate Diol-Boron Ester

Covalent substrate adducts could be observed for 2-formylphenylboronic acid and N-tert-butylhydroxylamine treated sample as summarized in the table below, substantiating the proposed mechanism for boronate ester protection (see Table 6).

TABLE 6
Observed m/z Values. Interpretation, and Estimated
Quantification of 2-FBPA/N-Hydroxylamine Adduct(s)

| Entry | m/z | | Ratio (B:C:D) |
|---|---|---|---|
| A | 3084 | native peptide | |
| B | 3262 | (gluconoylated peptide) | ~53% |
| C | 3447 | (gluconoylated peptide + 1 adduct) | ~35% |
| D | 3632 | (gluconoylated peptide + 2 adducts) | ~11% |

The expected mass increase for a single adduct is ˜185 Da. Relative percentages were calculated as TIC for a specific species divided by the sum of all species that can participate in the reaction, i.e., Ratio (%)=TIC (B or C or D)/(sum of TICs for B, C and D), where TIC is “Total Ion Count” from the MALDI-MS experiment

### Proposed Mechanism

Reaction Scheme. Proposed Mechanism for Trapping the Diol-Ester.

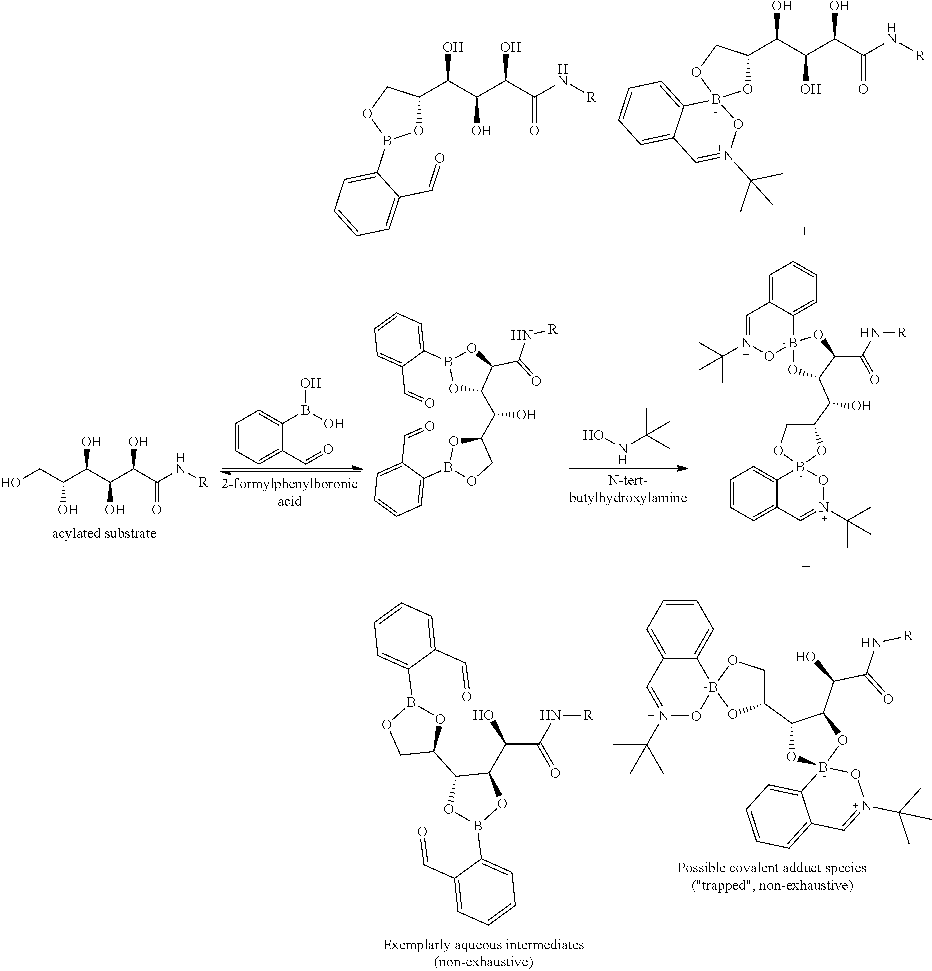

### Example 20

### Method for Covalently Trapping a Boron-Diol Ester of a Substrate Previously Acetylated with a Handle-Substituted Lactone and Treated with 2-Formylphenylboronc Acid

Having demonstrated trapping of a gluconoylated substrate, we next tested trapping of the boronate-diol ester of the same peptide previously treated with a handle-substituted 6-azido-6-deoxy-glucono-1,5-lactone. Lyophilized N-terminally azido-gluconylated peptide PP13 was resuspended to 200 μM in several buffers: 50 mM potassium phosphate (pH 6.8), 50 mM HEPES/NaOH (pH 7.5), and 50 mM ammonium bicarbonate (pH 8.44). To elucidate the importance of order and time, aliquots of 35 μL peptide solution were reacted sequentially with 4.3 μL of each reagent at 20 mM stock concentration (2-2.5 mM final concentration). After the indicated time, 4.3 μL of the second reagent at 20 mM were added (2-2.5 mM final concentration). N-tert-butylhydroxylamine hydrochloride was obtained from Sigma (CAS 57497-39-9, Cat. No. 194751). Reactions were conducted at room temperature, and samples (5 μL) were withdrawn at the indicated time, acidified with acetic acid (5 μL of 20% v/v solution), C18 ZipTip purified and analyzed by MALDI-TOF-MS.

### Results

The method could also be used to covalently trap an acylated substrate that was modified with a handle-substituted carbohydrate lactone according to the disclosure, e.g., treated with 6-azido-6-deoxy-D-glucono-1,5-lactone. Covalent adducts at the expected m/z value (+185 vs. acylated) were observed in all conditions tested, except for pH 6.8 with first addition of N-tert-butylhydroxylamine (Table 8).

TABLE 8

| | | | | | relative to all species (%) | | | relative to all species (%) | |
|---|---|---|---|---|---|---|---|---|---|
| | | | | Time waited | | | 6-AGDL | | |
| | First | Time before | Second | after addition | | | acylated | | 6-AGDL |
| | reagent | addition of | reagent | of second | Native | 6-AGDL | and | 6-AGDL | acylated and |
| PH | added | second reagent | added | reagent | peptide | acylated | trapped | acylated | trapped |
| 6.8 | 2FPBA | 90 min | NTBHA | 60 | 13.1 | 49.8 | 37.1 | 57.3 | 42.7 |
| 6.8 | NTBHA | 2 min | 2FPBA | 150 | 17.4 | 82.5 | 0.1 | 99.9 | 0.1 |
| 6.8 | 2FPBA | 2 min | NTBHA | 150 | 19.1 | 63.4 | 17.5 | 78.3 | 21.7 |
| 6.8 | — | — | — | — | 13.9 | 86 | 0.1 | 99.9 | 0.1 |
| 7.5 | 2FPBA | 90 min | NTBHA | 60 | 13.1 | 73.5 | 13.4 | 84.6 | 15.4 |
| 7.5 | NTBHA | 2 min | 2FPBA | 150 | 23.3 | 63.4 | 13.3 | 82.7 | 17.3 |
| 7.5 | 2FPBA | 2 min | NTBHA | 150 | 11.5 | 76 | 12.5 | 85.9 | 14.1 |
| 7.5 | — | — | — | — | 11.6 | 88.3 | 0 | 100 | 0 |
| 8.44 | 2FPBA | 90 min | NTBHA | 60 | 18.7 | 25.8 | 55.4 | 31.8 | 68.2 |
| 8.44 | NTBHA | 2 min | 2FPBA | 150 | 23 | 35.1 | 42 | 45.5 | 54.5 |
| 8.44 | 2FPBA | 2 min | NTBHA | 150 | 20.3 | 26.1 | 53.5 | 32.8 | 67.2 |
| 8.44 | — | — | — | — | 21.5 | 78.4 | 0.1 | 99.9 | 0.1 |

Abbreviations: 2-formylphenylboronic acid—2FPBA; N-tert-butylhydroxylamine—NTBHA. All species are: native peptide (3084 m/z), 6-AGDL acylated species (3287 m/z), and 6-AGDL acylated and trapped diol-ester (3472 m/z).

Best trapping yield results were obtained for a reaction conducted in slightly alkaline conditions (pH 8.4), first adding 2-formylphenylboronic acid, followed by addition of N-tert-butylhydroxylamine.

Adding N-tert-butylhydroxylamine first apparently caused faster reversal to the native, deacylated peptide—an effect that most pronounced at pH 7.5—to achieve highest trapping yield, it is thus preferable to first add the 2-formylphenylboronic acid to allow boronate-diol ester formation prior to addition of N-tert-butylhydroxylamine.

The time interval between adding the first and the second did not influence the reaction yield much in the neutral and alkaline condition, as such N-tert-butylhydroxylamine can be added relatively quickly after the addition of the boron compound in these conditions. In slightly acidic conditions (pH 6.8), pre-incubation with 2-formylphenylboronic acid for longer time (90 min) afforded higher yield compared to short incubation time (2 min), and may be the preferred method in this pH range.

Strikingly, only one adduct was observed for the 6-azido-6-deoxy-gluconyl substrate, whereas for the gluconoyl substrate up to two adducts were observed. This suggests that—without being bound by theory—for the previously tested gluconoyl substrate the most distal hydroxyl (C6) forms a boron-diol ester with C5. In the 6-azido gluconoyl substrate, this C6 hydroxyl is replaced by the azide group and thus cannot participate in C6-C5 diol ester. These results further suggest that by choosing the position of the (single or multiple position of handle-substitutions on a carbohydrate lactone), one can influence the number and position of adduct(s) that can form. That is for example, a 3-azido-3-deoxy-gluconoyl substrate would be expected to form only one adduct at C6-C5 (or C5-C4); whereas a 4-azido-4-deoxy-gluconoyl substrate could be capable of forming two adducts, one at C6-C5, and the other at C3-C2. It is understood that other carbohydrate lactones may have different hydroxyl-stereochemistries and thus may display different susceptible diol pairings.

Reaction Scheme. Proposed mechanism for trapping the diol-ester of an azido-handle-containing acylated substrate treated with 2-formylphenylboronic acid and an N-hydroxylamine.

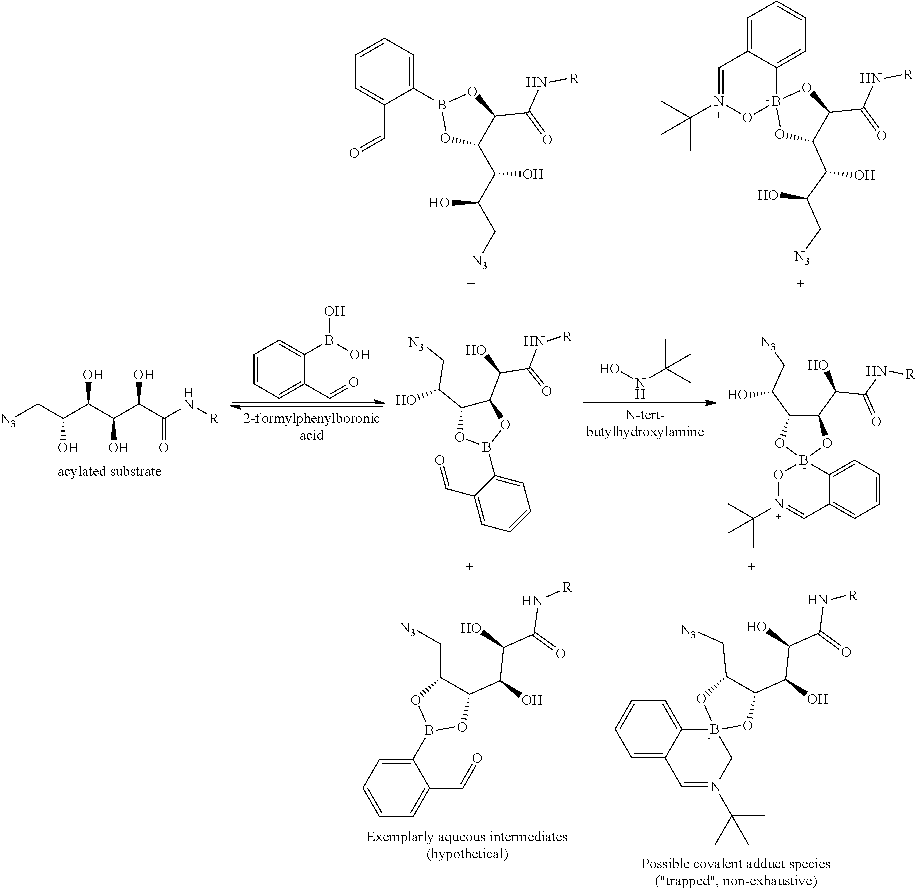

### BIBLIOGRAPHY

Aerni, Hans R., Mark A. Shifman, Svetlana Rogulina, Patrick O'Donoghue, and Jesse Rinehart. 2015. “Revealing the Amino Acid Composition of Proteins within an Expanded Genetic Code.” Nucleic Acids Research 43 (2): e8
Ahad, Ali M., Stephanie M. Jensen, and John C. Jewett. 2013. ‘A Traceless Staudinger Reagent To Deliver Diazirines.’ Organic Letters 15 (19): 5060-63. https://doi.org/10.1021/o1402404n
Aon, J. C., R. J. Caimi, A. H. Taylor, Q. Lu, F. Oluboyede, J. Dally, M. D. Kessler, et al. 2008. “Suppressing Posttranslational Gluconoylation of Heterologous Proteins by Metabolic Engineering of Escherichia Coli.” Applied and Environmental Microbiology 74 (4): 950-58
Apps, James, Andrew Livingston, Mark Parrett, Ryan Pounder, Paul Taylor, and Andrew Turner. 2014. ‘Racemisation of 1-Arylethylamines with Shvo-Type Organoruthenium Catalysts.’ Synlett 25 (10): 1391-94. https://doi.org/10.1055/s-0033-1341228
Augé, Claudine, Serge David, and Annie Malleron. 1989. ‘An Inexpensive Route to 2-Azido-2-Deoxy-d-Mannose and Its Conversion into an Azido Analog of N-Acetylneuraminic Acid.’ Carbohydrate Research 188 (June): 201-5. https://doi.org/10.1016/0008-6215(89)84071-6;
Ayers, Benjamin J., Nigel Ngo, Sarah F. Jenkinson, R. Fernando Martinez, Yousuke Shimada, Isao Adachi, Alexander C. Weymouth-Wilson, Atsushi Kato, and George W. J. Fleet. 2012. ‘Glycosidase Inhibition by All 10 Stereoisomeric 2,5-Dideoxy-2,5-Iminohexitols Prepared from the Enantiomers of Glucuronolactone.’ The Journal of Organic Chemistry 77 (18): 7777-92. https://doi.org/10.1021/jo301243s;
Baskin, J. M., and Bertozzi, C. R. 2010. “Copper-Free Click Chemistry: Bioorthogonal Reagents for Tagging Azides.” Aldrichimica acta 43(1):15-23
Barker, S. A., E. J. Bourne, R. M. Pinkard, and D. H. Whiffen. 1958. INFRARED SPECTRA OF CARBOHYDRATES-DIFFERENTIATION OF GAMMA-LACTONE AND DELTA-LACTONE OF ALDONIC ACIDS. 22. SOC CHEMICAL INDUSTRY 14 BELGRAVE SQUARE, LONDON SW1X 8PS, ENGLAND
Berger, Andreas, Anna de Raadt, Günther Gradnig, Monika Grasser, Harald Löw, and Arnold E. Stütz. 1992. ‘A Novel Synthetic Application of Glucose Isomerase: Quantitative Conversion of D-Glucose and L-Idofuranose, Derivatives Modified at Position Five into the Corresponding D-Fructo- and L-Sorbopyranoses.’ Tetrahedron Letters 33 (47): 7125-28. https://doi.org/10.1016/50040-4039(00)60853-5
Berkel, Sander S. van, A. (Ton) J. Dirks, Marjoke F. Debets, Floris L. van Delft, Jeroen J. L. M. Cornelissen, Roeland J. M. Nolte, and Floris P. J. T. Rutjes. 2007. ‘Metal-Free Triazole Formation as a Tool for Bioconjugation.’ ChemBioChem 8 (13): 1504-8. https://doi.org/10.1002/cbic.200700278
Bierenstiel, Matthias, and Marcel Schlaf 2004. “δ-Galactonolactone: Synthesis, Isolation, and Comparative Structure and Stability Analysis of an Elusive Sugar Derivative.” European Journal of Organic Chemistry 2004 (7): 1474-81
Bols, Mikael, Inge Lundt, Dorota Haber, Kálmán Burger, Harri Lönnberg, Jan-Eric Berg, Mihály Bartók, István Pelczer, and György Dombi. 1988. ‘Alpha-Amino Polyhydroxy Tetronic and Pentonic Acids from Bromodeoxyaldonolactones.’ Acta Chemica Scandinavica 42b: 67-74. https://doi.org/10.3891/acta.chem.scand.42b-0067
Brittain, William D G, Brette M. Chapin, Wenlei Zhai, Vincent M. Lynch, Benjamin R. Buckley, Eric V. Anslyn and John S. Fossey, 2016. “The Bull-James assembly as a chiral auxiliary and shift reagent in kinetic resolution of alkyne amines by the CuAAC reaction.” Organic & Biomolecular Chemistry 14(46):10778-10782
Buldun, Can M., Jisoo X. Jean, Michael R. Bedford, and Mark Howarth. 2018. “SnoopLigase Catalyzes Peptide-Peptide Locking and Enables Solid-Phase Conjugate Isolation.” Journal of the American Chemical Society 140 (8): 3008-18
Campo, Vanessa L., Renata Sesti-Costa, Zumira A. Carneiro, Joao S. Silva, Sergio Schenkman, and Ivone Carvalho. 2012. ‘Design, Synthesis and the Effect of 1,2,3-Triazole Sialylmimetic Neoglycoconjugates on Trypanosoma Cruzi and Its Cell Surface Trans-Sialidase.’ Bioorganic & Medicinal Chemistry 20 (1): 145-56. https://doi.org/10.1016/j.bmc.2011.11.022
Cao, Yuan, Giang K. T. Nguyen, Samuel Chuah, James P. Tam, and Chuan-Fa Liu. 2016. “Butelase-Mediated Ligation as an Efficient Bioconjugation Method for the Synthesis of Peptide Dendrimers.” Bioconjugate Chemistry 27 (11): 2592-96
Cesari, Cristiana. 2016. ‘Novel Ruthenium Complexes for Bifunctional Catalysis: The Dual Role of Cyclopentadienone and N-Heterocyclic Carbene.’ https://doi.org/10.6092/UNIBO/AMSDOTTORATO/7525
Chaveriat, Ludovic, Imane Stasik, Gilles Demailly, and Daniel Beaupere. 2004. ‘Improved Synthesis of 6-Amino-6-Deoxy-d-Galactono-1,6-Lactam and d-Mannono-1,6-Lactam from Corresponding Unprotected d-Hexono-1,4-Lactones.’ Tetrahedron 60 (9): 2079-81. https://doi.org/10.1016/j.tet.2003.12.062
Chaveriat, Ludovic, Imane Stasik, Gilles Demailly, and Daniel Beaupere. 2006. ‘The Direct Synthesis of 6-Amino-6-Deoxyaldonic Acids as Monomers for the Preparation of Polyhydroxylated Nylon 6.’ Tetrahedron: Asymmetry 17 (9): 1349-54. https://doi.org/10.1016/j.tetasy.2006.04.018
Conley, Brian L., Megan K. Pennington-Boggio, Emine Boz, and Travis J. Williams. 2010. ‘Discovery, Applications, and Catalytic Mechanisms of Shvo's Catalyst.’ Chemical Reviews 110 (4): 2294-2312. https://doi.org/10.1021/cr9003133
Cubas, Rafael, Sheng Zhang, Sunkuk Kwon, Eva M. Sevick-Muraca, Min Li, Changyi Chen, and Qizhi Yao. 2009. “Virus-like Particle (VLP) Lymphatic Trafficking and Immune Response Generation After Immunization by Different Routes:” Journal of Immunotherapy 32 (2): 118-28
Datta, Deepshikha, Pin Wang, Isaac S. Carrico, Stephen L. Mayo, and David A. Tirrell. 2002. “A Designed Phenylalanyl-TRNA Synthetase Variant Allows Efficient in Vivo Incorporation of Aryl Ketone Functionality into Proteins.” Journal of the American Chemical Society 124 (20): 5652-53
Davis, Lloyd, and Jason W. Chin. 2012. “Designer Proteins: Applications of Genetic Code Expansion in Cell Biology.” Nature Reviews Molecular Cell Biology 13 (3): 168
deGruyter, Justine N., Lara R. Malins, and Phil S. Baran. 2017. “Residue-Specific Peptide Modification: A Chemist's Guide.” Biochemistry 56 (30): 3863-73
Derewenda, Zygmunt S. 2004. “The Use of Recombinant Methods and Molecular Engineering in Protein Crystallization.” Methods (San Diego, Calif.) 34 (3): 354-63
Destito, Paolo, José R. Couceiro, Hélio Faustino, Fernando López, and José L. Mascarenas. 2017. ‘Ruthenium-Catalyzed Azide-Thioalkyne Cycloadditions in Aqueous Media: A Mild, Orthogonal, and Biocompatible Chemical Ligation.’ Angewandte Chemie International Edition 56 (36): 10766-70. https://doi.org/10.1002/anie.201705006
Dharuman, Suresh, Yichen Wang, and David Crich. 2016. ‘Alternative Synthesis and Antibacterial Evaluation of 1,5-Dideoxy-1,5-Imino-1-Rhamnitol.’ Carbohydrate Research 419 (January): 29-32. https://doi.org/10.1016/j.carres.2015.10.015
Dill, Kilian, Marsha E. Daman, Elaine Decoster, Jean M. Lacombe, and André A. Pavia. 1985. ‘Stereochemistry of Gd3+ and Mn2+ Interactions with D-Gluconamide Derivatives by 13C NMR Spectroscopy.’ Inorganica Chimica Acta 106 (4): 203-8. https://doi.org/10.1016/50020-1693(00)82270-7
Dommerholt, Jan, Floris P. J. T. Rutjes, and Floris L. van Delft. 2016. ‘Strain-Promoted 1,3-Dipolar Cycloaddition of Cycloalkynes and Organic Azides.’ Topics in Current Chemistry 374 (2): 16. https://doi.org/10.1007/s41061-016-0016-4
Duffy, Lorna A., Hiroshi Matsubara, and David J. Procter. 2008. ‘A Ring Size-Selective Reduction of Lactones Using SmI₂ and H₂O.’ Journal of the American Chemical Society 130 (4): 1136-37. https://doi.org/10.1021/ja078137d
Dutta, Indranil, Abir Sarbajna, Pragati Pandey, S. M. Wahidur Rahaman, Kuldeep Singh, and Jitendra K. Bera. 2016. ‘Acceptorless Dehydrogenation of Alcohols on a Diruthenium(II,II) Platform.’ Organometallics 35 (10): 1505-13. https://doi.org/10.1021/acs.organomet.6b00085
Falentin, Céline, Daniel Beaupère, Gilles Demailly, and Imane Stasik. 2007. ‘A Concise Synthesis of 5-Amino-5-Deoxyaldonic Acids as Monomers for the Preparation of Nylon 5.’ Carbohydrate Research 342 (18): 2807-9. https://doi.org/10.1016/j.carres.2007.09.008
Fierer, Jacob O., Gianluca Veggiani, and Mark Howarth. 2014. “SpyLigase Peptide-Peptide Ligation Polymerizes Affibodies to Enhance Magnetic Cancer Cell Capture.” Proceedings of the National Academy of Sciences of the United States of America 111 (13): E1176-1181
Fitz, Wolfgang, Jan-Robert Schwark, and Chi-Huey Wong. 1995. ‘Aldotetroses and C(3)-Modified Aldohexoses as Substrates for N-Acetylneuraminic Acid Aldolase: A Model for the Explanation of the Normal and the Inversed Stereoselectivity.’ The Journal of Organic Chemistry 60 (12): 3663-70. https://doi.org/10.1021/jo00117a016
Fujita, Ken-ichi, Nobuhide Tanino, and Ryohei Yamaguchi. 2007. ‘Ligand-Promoted Dehydrogenation of Alcohols Catalyzed by Cp*Ir Complexes. A New Catalytic System for Oxidant-Free Oxidation of Alcohols.’ Organic Letters 9 (1): 109-11. https://doi.org/10.1021/o1062806v
Gan, Qinglei, and Chenguang Fan. 2017. “Increasing the Fidelity of Noncanonical Amino Acid Incorporation in Cell-Free Protein Synthesis.” Biochimica et Biophysica Acta (BBA)—General Subjects 1861 (11): 3047-52
Geoghegan, K. F., H. B. Dixon, P. J. Rosner, L. R. Hoth, A. J. Lanzetti, K. A. Borzilleri, E. S. Marr, et al. 1999. “Spontaneous Alpha-N-6-Phosphogluconoylation of a ‘His Tag’ in Escherichia Coli: The Cause of Extra Mass of 258 or 178 Da in Fusion Proteins.” Analytical Biochemistry 267 (1): 169-84
Geoghegan, Kieran F. 2016. “Modification of Amino Groups: Modification of Amino Groups.” In Current Protocols in Protein Science, edited by John E. Coligan, Ben M. Dunn, David W. Speicher, and Paul T. Wingfield, 15.2.1-15.2.20. Hoboken, N.J., USA: John Wiley & Sons, Inc
Grimsley, Gerald R., J. Martin Scholtz, and C. Nick Pace. 2008. “A Summary of the Measured p K Values of the Ionizable Groups in Folded Proteins.” Protein Science, NA-NA. https://doi.org/10.1002/pro.19
Guthrie, R. D., and D. Murphy. 1963. ‘1009. Nitrogen-Containing Carbohydrate Derivatives. Part IV. Some Azido- and Epimino-Sugars.’ Journal of the Chemical Society (Resumed), 5288. https://doi.org/10.1039/jr9630005288
Györgyde\grave{s}k, Zoltàn, and Làszlò Szilàgyi. 1987. ‘Einfache Synthesen der anomeren, an C-6 modifizierten Galacto- und Glucopyranosylazide.’ Liebigs Annalen der Chemie 1987 (3): 235-41. https://doi.org/10.1002/jlac.198719870314
Hamagami, Hiroki, Motofumi Kumazoe, Yoshiki Yamaguchi, Shinichiro Fuse, Hirofumi Tachibana, and Hiroshi Tanaka. 2016. ‘6-Azido-6-Deoxy-L-Idose as a Hetero-Bifunctional Spacer for the Synthesis of Azido-Containing Chemical Probes.’ Chemistry—A European Journal 22 (36): 12884-90. https://doi.org/10.1002/chem.201602044
Hanessian, S. 1966. ‘Synthesis of Hydroxypiperidines from Carbohydrate Precursors’, Chem. Ind. no. 51: 2126-27
Hanessian, S. 1969. “Sugar Lactams. III. Synthesis of Five-,Six-, and Seven-Membered Analogs.” The Journal of Organic Chemistry 34 (3): 675-81. https://doi.org/10.1021/jo01255a042
Hanessian, Stephen, Guobin Huang, Caroline Chenel, Roger Machaalani, and Olivier Loiseleur. 2005. ‘Total Synthesis of N-Malayamycin A and Related Bicyclic Purine and Pyrimidine Nucleosides.’ The Journal of Organic Chemistry 70 (17): 6721-34. https://doi.org/10.1021/jo050727b
Harris, Karen S., Thomas Durek, Quentin Kaas, Aaron G. Poth, Edward K. Gilding, Brendon F. Conlan, Ivana Saska, et al. 2015. “Efficient Backbone Cyclization of Linear Peptides by a Recombinant Asparaginyl Endopeptidase.” Nature Communications 6 (1): 10199
Hassan, M. E., and J. T. Slama. 1992. ‘Synthesis of Potential Inhibitors of Glycosidases and Glycohydrolases. 1. 1, 4-Imino-1, 4-Dideoxyribose.’ Chemical Papers 46 (4): 261-265
Hauser, Christina T., and Roger Y. Tsien. 2007. “A Hexahistidine-Zn2+-Dye Label Reveals STIM1 Surface Exposure.” Proceedings of the National Academy of Sciences of the United States of America 104 (10): 3693-97
He, Dongmei, and Istvin T. Horvith. 2017. “Application of Silica-Supported Shvo's Catalysts for Transfer Hydrogenation of Levulinic Acid with Formic Acid.” Journal of Organometallic Chemistry 847 (October): 263-69. https://doi.org/10.1016/j.jorganchem.2017.05.039
Hermanson, Greg T. 2013. Bioconjugate Techniques. Third edition. London; Waltham, Mass.: Elsevier/AP
Hirata, R., Y. Ohsumk, A. Nakano, H. Kawasaki, K. Suzuki, and Y. Anraku. 1990. “Molecular Structure of a Gene, VMA1, Encoding the Catalytic Subunit of H(+)-Translocating Adenosine Triphosphatase from Vacuolar Membranes of Saccharomyces Cerevisiae.” The Journal of Biological Chemistry 265 (12): 6726-33
Hobbs, J. B., and F. Eckstein. 1977. ‘A General Method for the Synthesis of 2′-Azido-2′-Deoxy- and 2′-Amino-2′-Deoxyribofuranosyl Purines.’ The Journal of Organic Chemistry 42 (4): 714-19. https://doi.org/10.1021/jo00424a031
Huang, Jie, Wei-Lin Dai, and Kangnian Fan. 2008. ‘Support Effect of New Au/FeO x Catalysts in the Oxidative Dehydrogenation of α,ω-Diols to Lactones.’ The Journal of Physical Chemistry C 112 (41): 16110-17. https://doi.org/10.1021/jp8043913
Hudlicky, Tomas, Thomas Nugent, and William Griffith. 1994. ‘Chemoenzymic Synthesis of D-Erythro- and L-Threo-C18-Sphingosines.’ The Journal of Organic Chemistry 59 (26): 7944-46. https://doi.org/10.1021/jo00105a002
Hudson, C. S., and H. S. Isbell. 1929. “RELATIONS BETWEEN ROTATORY POWER AND STRUCTURE IN THE SUGAR GROUP. XIX. IMPROVEMENTS IN THE PREPARATION OF ALDONIC ACIDS 1.” Journal of the American Chemical Society 51 (7): 2225-29
Isbell, H. S., and H. L. Frush. 1933. ‘Preparation and Properties of Aldonic Acids and Their Lactones and Basic Calcium Salts.’ Bureau of Standards Journal of Research 11 (5): 649. https://doi.org/10.6028/jres.011.043
Jackson, M. A., E. K. Gilding, T. Shafee, K. S. Harris, Q. Kaas, S. Poon, K. Yap, et al. 2018. “Molecular Basis for the Production of Cyclic Peptides by Plant Asparaginyl Endopeptidases.” Nature Communications 9 (1): 2411
Jacob, E., and R. Unger. 2007. “A Tale of Two Tails: Why Are Terminal Residues of Proteins Exposed?” Bioinformatics 23 (2): e225-30
Jain, Nareshkumar, Sean W. Smith, Sanjeevani Ghone, and Bruce Tomczuk. 2015. “Current ADC Linker Chemistry.” Pharmaceutical Research 32 (11): 3526-40
Kanekiyo, Masaru, Daniel Ellis, and Neil P King. 2019. “New Vaccine Design and Delivery Technologies.” The Journal of Infectious Diseases, January. https://doi.org/10.1093/infdis/jiy745
Keeble, Anthony H., Anusuya Banerjee, Matteo P. Ferla, Samuel C. Reddington, Irsyad N. A. Khairil Anuar, and Mark Howarth. 2017. ‘Evolving Accelerated Amidation by SpyTag/SpyCatcher to Analyze Membrane Dynamics.’ Angewandte Chemie (International Ed. in English) 56 (52): 16521-25. https://doi.org/10.1002/anie.201707623
Kefurt, Karel, Karel Čapek, Zdeňka Kefurtová, and Jiři Jarý. 1986. ‘Preparation of 6-Amino-6-Deoxy-D-Altronic Acid and Their Derivatives.’ Collection of Czechoslovak Chemical Communications 51 (2): 391-400. https://doi.org/10.1135/cccc19860391
Kefurt, Karel, Zdeňka Kefurtová, and Jiři Jarý. 1988. ‘6-Amino-6-Deoxyhexonolactams.’ Collection of Czechoslovak Chemical Communications 53 (8): 1795-1805
Khedri, Zahra, Yanhong Li, Saddam Muthana, Musleh M. Muthana, Ching-Wen Hsiao, Hai Yu, and Xi Chen. 2014. ‘Chemoenzymatic Synthesis of Sialosides Containing C7-Modified Sialic Acids and Their Application in Sialidase Substrate Specificity Studies.’ Carbohydrate Research 389 (May): 100-111. https://doi.org/10.1016/j.carres.2014.02.021
Kowalska, Kornelia, Agnieszka Adamczyk-Woźniak, Patrycja Gajowiec, Błażej Gierczyk, Ewa Kaczorowska, Łukasz Popenda, Grzegorz Schroeder, Artur Sikorski, Andrzej Sporzynski. 2016. “Fluoro-substituted 2-formylphenylboronic acids: Structures, properties and tautomeric equilibria.” Journal of Fluorine Chemistry 187: 1-8
Kumar, Ramar Arun, Manas R. Pattanayak, Expédite Yen-Pon, Jijy Eliyan, Karine Porte, Sabrina Bernard, Margaux Riomet, Pierre Thuéry, Davide Audisio, and Frédéric Taran. 2019. ‘Strain-Promoted 1,3-Dithiolium-4-olates-Alkyne Cycloaddition.’ Angewandte Chemie 131 (41): 14686-90. https://doi.org/10.1002/ange.201908052
Kunjapur, Aditya M., Devon A. Stork, Erkin Kuru, Oscar Vargas-Rodriguez, Matthieu Landon, Dieter Söll, and George M. Church. 2018. “Engineering Posttranslational Proofreading to Discriminate Nonstandard Amino Acids.” Proceedings of the National Academy of Sciences 115 (3): 619-24
Lajoie, M. J., A. J. Rovner, D. B. Goodman, H.-R. Aerni, A. D. Haimovich, G. Kuznetsov, J. A. Mercer, et al. 2013. “Genomically Recoded Organisms Expand Biological Functions.” Science 342 (6156): 357-60
Lang, Kathrin, and Jason W. Chin. 2014. “Cellular Incorporation of Unnatural Amino Acids and Bioorthogonal Labeling of Proteins.” Chemical Reviews 114 (9): 4764-4806
Li, Dachao, Jianwei Wu, Peng Wu, Yuan Lin, Yingjuan Sun, Rui Zhu, Jia Yang, and Kexin Xu. 2015. ‘Affinity Based Glucose Measurement Using Fiber Optic Surface Plasmon Resonance Sensor with Surface Modification by Borate Polymer.’ Sensors and Actuators B: Chemical 213 (July): 295-304
Li, L., and Zhang, Z. 2016 “Development and Applications of the Copper-Catalyzed Azide-Alkyne Cycloaddition (CuAAC) as a Bioorthogonal Reaction.” Molecules 21(10). pii: E1393
Li, Zibo, Hancheng Cai, Matthew Hassink, Melissa L. Blackman, Richard C. D. Brown, Peter S. Conti, and Joseph M. Fox. 2010. ‘Tetrazine-Trans-Cyclooctene Ligation for the Rapid Construction of 18F Labeled Probes.’ Chemical Communications 46 (42): 8043. https://doi.org/10.1039/c0cc03078c
Ligeour, Caroline, Lucie Dupin, Anthony Angeli, Gerard Vergoten, Sébastien Vidal, Albert Meyer, Eliane Souteyrand, Jean-Jacques Vasseur, Yann Chevolot, and Frangois Morvan. 2015. ‘Importance of Topology for Glycocluster Binding to Pseudomonas Aeruginosa and Burkholderia Ambifaria Bacterial Lectins.’ Organic & Biomolecular Chemistry 13 (46): 11244-54. https://doi.org/10.1039/C50B01445J
Long, Daniel D., Rebecca J. E. Stetz, Robert J. Nash, Daniel G. Marquess, Janet D. Lloyd, Ana L. Winters, Naoki Asano, and George W. J. Fleet. 1999. ‘Open-Chain Acetonides of D-Galactono-1,4-Lactone as Starting Materials for Pyrrolidines, Azepanes and 5-Azidomethyltetrahydrofuran-2-Carboxylates: Monomers for Polyhydroxylated Nylon and for Tetrahydrofuran Carbopeptoids.’ Journal of the Chemical Society, Perkin Transactions 1, no. 8: 901-8. https://doi.org/10.1039/a809807g
Lowary, Todd L., and Ole Hindsgaul. 1994. ‘Recognition of Synthetic O-Methyl, Epimeric, and Amino Analogues of the Acceptor α-L-Fucp-(1→2)-β-D-Galp-OR Glycosyltransferases.’ Carbohydrate Research 251 (January): 33-67. https://doi.org/10.1016/0008-6215(94)84275-2
Luliński, Sergiusz, Izabela Madura, Janusz Serwatowski, Halina Szatylowicz and Janusz Zachara. 2007. “A tautomeric equilibrium between functionalized 2-formylphenylboronic acids and corresponding 1,3-dihydro-1,3-dihydroxybenzo[c][2,1]oxaboroles.” New Journal of Chemistry 31: 144-154
Manolova, Vania, Anna Flace, Monika Bauer, Katrin Schwarz, Philippe Saudan, and Martin F. Bachmann. 2008. “Nanoparticles Target Distinct Dendritic Cell Populations According to Their Size.” European Journal of Immunology 38 (5): 1404-13
Martos-Maldonado, Manuel C., Christian T. Hjuler, Kasper K. Sorensen, Mikkel B. Thygesen, Jakob E. Rasmussen, Klaus Villadsen, Søren R. Midtgaard, Stefan Kol, Sanne Schoffelen, and Knud J. Jensen. 2018. “Selective N-Terminal Acylation of Peptides and Proteins with a Gly-His Tag Sequence.” Nature Communications 9 (1). https://doi.org/10.1038/s41467-018-05695-3
Mazmanian, S. K. 1999. “Staphylococcus Aureus Sortase, an Enzyme That Anchors Surface Proteins to the Cell Wall.” Science 285 (5428): 760-63
McDevitt, Jason P., and Peter T. Lansbury. 1996. ‘Glycosamino Acids: New Building Blocks for Combinatorial Synthesis.’ Journal of the American Chemical Society 118 (16): 3818-28. https://doi.org/10.1021/ja9525622
McKay, Craig S., and M. G. Finn. 2014. ‘Click Chemistry in Complex Mixtures: Bioorthogonal Bioconjugation.’ Chemistry & Biology 21 (9): 1075-1101. https://doi.org/10.1016/j.chembiol.2014.09.002
Meadows, Margaret K, Emily K Roesner, Vincent M. Lynch, Tony D. James and Eric V. Anslyn. 2017. “Boronic Acid Mediated Coupling of Catechols and N-Hydroxylamines: A Bioorthogonal Reaction to Label Peptides.” Organic Letters 19(12):3179-3182
Artem Melman. 2010. “Synthesis of Hydroxylamines.” PATAI'S Chemistry of Functional Groups. https://doi.org/10.1002/9780470682531.pat0457
Meyer, Jan-Philip, Pierre Adumeau, Jason S. Lewis, and Brian M. Zeglis. 2016. ‘Click Chemistry and Radiochemistry: The First 10 Years.’ Bioconjugate Chemistry 27 (12): 2791-2807. https://doi.org/10.1021/acs.bioconjchem.6b00561
Michlik, Stefan, and Rhett Kempe. 2013. ‘A Sustainable Catalytic Pyrrole Synthesis.’ Nature Chemistry 5 (2): 140-44. https://doi.org/10.1038/nchem.1547
Miclet, Emeric, Véronique Stoven, Paul A. M. Michels, Fred R. Opperdoes, Jean-Yves Lallemand, and Francis Duffieux. 2001. ‘NMR Spectroscopic Analysis of the First Two Steps of the Pentose-Phosphate Pathway Elucidates the Role of 6-Phosphogluconolactonase.’ Journal of Biological Chemistry 276 (37): 34840-46. https://doi.org/10.1074/jbc.M105174200
Midtgaard, Søren Roi, Martin Cramer Pedersen, Jacob Judas Kain Kirkensgaard, Kasper Kildegaard Sørensen, Kell Mortensen, Knud J. Jensen, and Lise Arleth. 2014. “Self-Assembling Peptides Form Nanodiscs That Stabilize Membrane Proteins.” Soft Matter 10 (5): 738-52
Mitsudome, Takato, Akifumi Noujima, Tomoo Mizugaki, Koichiro Jitsukawa, and Kiyotomi Kaneda. 2009. ‘Supported Gold Nanoparticles as a Reusable Catalyst for Synthesis of Lactones from Diols Using Molecular Oxygen as an Oxidant under Mild Conditions.’ Green Chemistry 11 (6): 793. https://doi.org/10.1039/b900576e
Morais, Marta P. Pereira, John S. Fossey, Tony D. James, and Jean M. H. van den Elsen. 2012. ‘Analysis of Protein Glycation Using Phenylboronate Acrylamide Gel Electrophoresis.’ In Protein Electrophoresis, edited by Biji T. Kurien and R. Hal Scofield, 869:93-109. Totowa, N.J.: Humana Press
Moreno, Karlos X., Crystal E. Harrison, Matthew E. Merritt, Zoltan Kovacs, Craig R. Malloy, and A. Dean Sherry. 2017. ‘Hyperpolarized δ-[1-¹³C]Gluconolactone as a Probe of the Pentose Phosphate Pathway.’ NMR in Biomedicine 30 (6): e3713. https://doi.org/10.1002/nbm.3713
Mori, Miwako, Yumiko Washioka, Takao Urayama, Kagari Yoshiura, Katsumi Chiba, and Yoshio Ban. 1983. ‘Palladium-Catalyzed Carbonylation. A New Synthesis of .Alpha.-Methylene .Gamma.-, .Delta.-, .Epsilon.-Lactams and Lactones Including Bicyclic Lactams of Pyrrolizidine and Indolizidine Skeletons.’ The Journal of Organic Chemistry 48 (22): 4058-67. https://doi.org/10.1021/jo00170a037
Müller, Bernhard, Markus Blaukopf, Andreas Hofinger, Alla Zamyatina, Helmut Brade, and Paul Kosma. 2010. ‘Efficient Synthesis of 4-Amino-4-Deoxy-1-Arabinose and Spacer-Equipped 4-Amino-4-Deoxy-1-Arabinopyranosides by Transglycosylation Reactions.’ Synthesis 2010 (18): 3143-51. https://doi.org/10.1055/s-0030-1258174
Musa, Sanaa, Irina Shaposhnikov, Shmuel Cohen, and Dmitri Gelman. 2011. ‘Ligand-Metal Cooperation in PCP Pincer Complexes: Rational Design and Catalytic Activity in Acceptorless Dehydrogenation of Alcohols.’ Angewandte Chemie International Edition 50 (15): 3533-37. https://doi.org/10.1002/anie.201007367
Nagy, Adrienn, Barbara Csordás, Virág Zsoldos-Mády, Istvin Pinter, Viktor Farkas, and András Perczel. 2017. ‘C-3 Epimers of Sugar Amino Acids as Foldameric Building Blocks: Improved Synthesis, Useful Derivatives, Coupling Strategies.’ Amino Acids 49 (2): 223-40. https://doi.org/10.1007/s00726-016-2346-5
Nguyen, Giang K T, Shujing Wang, Yibo Qiu, Xinya Hemu, Yilong Lian, and James P Tam. 2014. “Butelase 1 Is an Asx-Specific Ligase Enabling Peptide Macrocyclization and Synthesis.” Nature Chemical Biology 10 (9): 732-38
Ning, Xinghai, Rinske P. Temming, Jan Dommerholt, Jun Guo, Daniel B. Ania, Marjoke F. Debets, Margreet A. Wolfert, Geert-Jan Boons, and Floris L. van Delft. 2010. ‘Protein Modification by Strain-Promoted Alkyne-Nitrone Cycloaddition.’ Angewandte Chemie International Edition 49 (17): 3065-68. https://doi.org/10.1002/anie.201000408
Noll, Stephan, Jan Reyelt, Thomas Rysiok, Roland Kellner, Detlef Güssow, Stefan Jäkel, Stefanie Hager, and Harald Kranz. 2013. “Gezielte Optimierung von Escherichia coli BL21(DE3): Heterologe Proteinexpression.” BIOspektrum 19 (2): 211-13
Oded, Kobi, Sanaa Musa, Dmitri Gelman, and Jochanan Blum. 2012. ‘Dehydrogenation of Alcohols under Ambient Atmosphere by a Recyclable Sol-Gel Encaged Iridium Pincer Catalyst.’ Catalysis Communications 20 (April): 68-70. https://doi.org/10.1016/j.catcom.2012.01.017
Pickens, Chad J., Stephanie N. Johnson, Melissa M. Pressnall, Martin A. Leon, and Cory J. Berkland. 2018. ‘Practical Considerations, Challenges, and Limitations of Bioconjugation via Azide-Alkyne Cycloaddition.’ Bioconjugate Chemistry 29 (3): 686-701
Pereira Morais, Marta P., Julia D. Mackay, Savroop K. Bhamra, J. Grant Buchanan, Tony D. James, John S. Fossey, and Jean M. H. van den Elsen. 2010. ‘Analysis of Protein Glycation Using Phenylboronate Acrylamide Gel Electrophoresis.’ PROTEOMICS 10 (1): 48-58
Pröschel, Marlene, Rainer Detsch, Aldo R. Boccaccini, and Uwe Sonnewald. 2015. “Engineering of Metabolic Pathways by Artificial Enzyme Channels.” Frontiers in Bioengineering and Biotechnology 3 (October). https://doi.org/10.3389/fbioe.2015.00168
Psurski, Mateusz, Agnieszka Łupicka-Słowik, Agnieszka Adamczyk-Woźniak, Joanna Wietrzyk and Andrzej Sporzyński. 2019. “Discovering simple phenylboronic acid and benzoxaborole derivatives for experimental oncology—phase cycle-specific inducers of apoptosis in A2780 ovarian cancer cells.” Investigational New Drugs 37(1):35-46
Ren, Chengfeng, Cedric E. Bobst, and Igor A. Kaltashov. 2019. ‘Exploiting His-Tags for Absolute Quantitation of Exogenous Recombinant Proteins in Biological Matrices: Ruthenium as a Protein Tracer.’ Analytical Chemistry 91 (11): 7189-98. https://doi.org/10.1021/acs.analchem.9b00504
Ren, Chengfeng, Cedric E. Bobst, and Igor A. Kaltashov. 2019. ‘Exploiting His-Tags for Absolute Quantitation of Exogenous Recombinant Proteins in Biological Matrices: Ruthenium as a Protein Tracer.’ Analytical Chemistry 91 (11): 7189-98. https://doi.org/10.1021/acs.analchem.9b00504
Roy, Arup, and Gangadhar J. Sanjayan. 2012. ‘Sugar-Amino Acid Cyclic Conjugates as Novel Conformationally Constrained Hydroxyethylamine Transition-State Isosteres.’ Tetrahedron Letters 53 (26): 3361-63. https://doi.org/10.1016/j.tetlet.2012.04.086
Rosen, Christian B, and Matthew B Francis. 2017. “Targeting the N Terminus for Site-Selective Protein Modification.” Nature Chemical Biology 13 (7): 697-705
Sadet, Aude, Emmanuelle M. M. Weber, Aditya Jhajharia, Dennis Kurzbach, Geoffrey Bodenhausen, Emeric Miclet, and Daniel Abergel. 2018. ‘Rates of Chemical Reactions Embedded in a Metabolic Network by Dissolution Dynamic Nuclear Polarisation NMR.’ Chemistry—A European Journal 24 (21): 5456-61. https://doi.org/10.1002/chem.201705520
Singh, Biswajit Kumar, Amit Kumar Yadav, Brijesh Kumar, A. Gaikwad, Sudhir Kumar Sinha, Vinita Chaturvedi, and Rama Pati Tripathi. 2008. ‘Preparation and Reactions of Sugar Azides with Alkynes: Synthesis of Sugar Triazoles as Antitubercular Agents.’ Carbohydrate Research 343 (7): 1153-62. https://doi.org/10.1016/j.carres.2008.02.013
Sinha, Sudhir K., and Keith Brew. 1980. ‘Synthesis of 4-Deoxy-d-Xylo-Hexose and 4-Azido-4-Deoxy-d-Glucose and Their Effects on Lactose Synthase.’ Carbohydrate Research 81 (2): 239-47. https://doi.org/10.1016/S0008-6215(00)85655-4
Spicer, C. D. and Davis, B. G. 2014. “Selective chemical protein modification.” Nature Communications. 5:4740
Spicer, Christopher D., E. Thomas Pashuck, and Molly M. Stevens. 2018. ‘Achieving Controlled Biomolecule-Biomaterial Conjugation.’ Chemical Reviews 118 (16): 7702-43. https://doi.org/10.1021/acs.chemrev.8b00253
Steffen, Wojtek, Fu Chong Ko, Jigar Patel, Victor Lyamichev, Thomas J. Albert, Jörg Benz, Markus G. Rudolph, et al. 2017. “Discovery of a Microbial Transglutaminase Enabling Highly Site-Specific Labeling of Proteins.” The Journal of Biological Chemistry 292 (38): 15622-35
Stöckmann, Henning, André A. Neves, Shaun Stairs, Kevin M. Brindle, and Finian J. Leeper. 2011. ‘Exploring Isonitrile-Based Click Chemistry for Ligation with Biomolecules.’ Organic & Biomolecular Chemistry 9 (21): 7303. https://doi.org/10.1039/clob06424j
Tipson, R Stuart. 1968. “Infrared Spectroscopy of Carbohydrates: A Review of the Literature,” June, 38
Tseng, Kuei-Nin T., Jeff W. Kampf, and Nathaniel K. Szymczak. 2013. ‘Base-Free, Acceptorless, and Chemoselective Alcohol Dehydrogenation Catalyzed by an Amide-Derived NNN -Ruthenium(II) Hydride Complex.’ Organometallics 32 (7): 2046-49. https://doi.org/10.1021/om4000677
Uriel, Clara, and Francisco Santoyo-González. 1999. ‘A Short and Efficient Synthesis of 1,5-Dideoxy-1,5-Imino-d-Galactitol (1-Deoxy-d-Galactostatin) and 1,5-Dideoxy-1,5-Imino-1-Altritol (1-Deoxy-1-Altrostatin) from d-Galactose.’ Synlett 1999 (5): 593-95
Vorm, Stefan van der. 2018. ‘Reactivity and Selectivity in Glycosylation Reactions.’ http://hdl.handle.net/1887/66126
Walaszek, Zbigniew, and Derek Horton. 1982. ‘Conformational Studies on Aldonolactones by N.M.R. Spectroscopy Conformations of d-Glucono-, d-Mannono-, d-Gulono- and d-Galactono-1,4-Lactone in Solution.’ Carbohydrate Research 105 (1): 131-43. https://doi.org/10.1016/S0008-6215(00)81859-5
Waßaszek, Zbigniew, Derek Horton, and Irena Ekiel. 1982. ‘Conformational Studies on Aldonolactones by n.m.r. Spectroscopy. Conformations of d-Glucono-1,5-Lactone and d-Mannono-1,5-Lactone in Solution.’ Carbohydrate Research 106 (2): 193-201. https://doi.org/10.1016/S0008-6215(00)81073-3
Wang, L., A. Brock, B. Herberich, and P. G. Schultz. 2001. “Expanding the Genetic Code of Escherichia Coli.” Science (New York, N.Y.) 292 (5516): 498-500
Worch, Marco, and Valentin Wittmann. 2008. ‘Unexpected Formation of Complex Bridged Tetrazoles via Intramolecular 1,3-Dipolar Cycloaddition of 1,2-O-Cyanoalkylidene Derivatives of 3-Azido-3-Deoxy-d-Allose.’ Carbohydrate Research 343 (12): 2118-29. https://doi.org/10.1016/j.carres.2007.10.034
Xie, Jianming, and Peter G. Schultz. 2006. “A Chemical Toolkit for Proteins—an Expanded Genetic Code.” Nature Reviews Molecular Cell Biology 7 (10): 775
Yan, Dan, Yan-Quan Wei, Hui-Chen Guo and Shi-Qi Sun. 2015. “The application of virus-like particles as vaccines and biological vehicles.” Applied Microbiology and Biotechnology 99(24):10415-32
Yang, Renliang, Yee Hwa Wong, Giang K T Nguyen, James P Tam, Julien Lescar, and Bin Wu. 2017. “Engineering a Catalytically Efficient Recombinant Protein Ligase.” Journal of the American Chemical Society 139 (15): 5351-58
Zakeri, Bijan, and Mark Howarth. 2010. “Spontaneous Intermolecular Amide Bond Formation between Side Chains for Irreversible Peptide Targeting.” Journal of the American Chemical Society 132 (13): 4526-27
Zaro, Balyn W., Anna R. Batt, Kelly N. Chuh, Marisol X. Navarro, and Matthew R. Pratt. 2017. ‘The Small Molecule 2-Azido-2-Deoxy-Glucose Is a Metabolic Chemical Reporter of 0-GlcNAc Modifications in Mammalian Cells, Revealing an Unexpected Promiscuity of O-GlcNAc Transferase.’ ACS Chemical Biology 12 (3): 787-94
Zhang, Yao, Cheng Chen, Subhash Chandra Ghosh, Yongxin Li, and Soon Hyeok Hong. 2010. ‘Well-Defined N-Heterocyclic Carbene Based Ruthenium Catalysts for Direct Amide Synthesis from Alcohols and Amines.’ Organometallics 29 (6): 1374-78. https://doi.org/10.1021/om901020h
Zhao, Jing, and John F. Hartwig. 2005. ‘Acceptorless, Neat, Ruthenium-Catalyzed Dehydrogenative Cyclization of Diols to Lactones.’ Organometallics 24 (10): 2441-46. https://doi.org/10.1021/om048983m
Zou, Yang, Wenjun Wang, Li Cai, Leilei Chen, Mengyang Xue, Xiaomei Zhang, Jie Shen, and Min Chen. 2012. ‘Substrate Specificity of Galactokinase from Streptococcus Pneumoniae TIGR4 towards Galactose, Glucose, and Their Derivatives.’ Bioorganic & Medicinal Chemistry Letters 22 (10): 3540-43. https://doi.org/10.1016/j.bmcl.2012.03.095

1. An N-terminally acylated protein, wherein the N-terminally acylated protein comprises formula (VIII), formula (VII) or formula (IX): 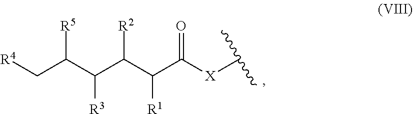
wherein:
(i) R¹, R², R³ and R⁵ are each independently hydrogen, a hydroxyl, a methyl, or a handle,
(ii) R⁴ is hydrogen, a hydroxyl, a methyl, a handle, a carboxyl, an ester, or an amide,
(iii) at least one of R¹, R², R³ and R⁴ is a handle,
(iv) one or none of R¹, R², R³ and R⁴ is a methyl; and
(v) X comprises the N-terminal amino acid residue of the protein; 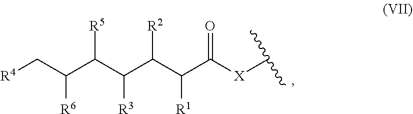
wherein:
(i) R¹, R², R³, R⁵ and R⁶ are each independently hydrogen, a hydroxyl, a methyl, or a handle,
(ii) R⁴ is hydrogen, a hydroxyl, a methyl, a handle, or a carboxyl,
(iii) at least one of R¹, R², R³ and R⁴ is a handle,
(iv) one or none of R¹, R², R³ and R⁴ is a methyl; and
(v) X comprises the N-terminal amino acid residue of the protein;
or 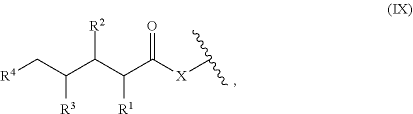
wherein:
(i) R¹, R² and R³ are each independently hydrogen, a hydroxyl, a methyl, or a handle,
(ii) R⁴ is a hydroxyl,
(iii) at least one of R¹, R², and R³ is a handle,
(iv) one or none of R¹, R², and R³ is a methyl; and
(v) X comprises the N-terminal amino acid residue of the protein.

2. The N-terminally acylated protein of claim 1, wherein the N-terminally acylated protein comprises formula (IV): 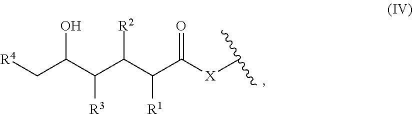
wherein:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide,
(iv) one or none of R¹, R², R³ and R⁴ is a methyl; and
(v) X comprises the N-terminal amino acid residue of the protein.

3. The N-terminally acylated protein of claim 1, wherein the N-terminally acylated protein comprises formula (X): 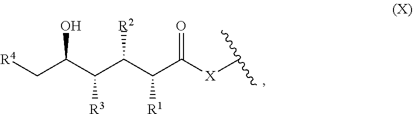
wherein:
(i) R⁴ is an azide, R³ is a hydroxyl, R² is a hydroxyl and R¹ is a hydroxyl;
(ii) R⁴ is a hydroxyl, R³ is an azide, R² is a hydroxyl and R¹ is a hydroxyl;
(iii) R⁴ is a hydroxyl, R³ is a hydroxyl, R² is an azide and R¹ is a hydroxyl; or
(iv) R⁴ is a hydroxyl, R³ is a hydroxyl, R² is a hydroxyl and R¹ is an azide.

4. The N-terminally acylated protein of claim 2, wherein R⁴ is an azide, R³ is a hydroxyl, R² is a hydroxyl and R¹ is a hydroxyl.

5. The N-terminally acylated protein of claim 1, wherein X comprises the amino acid sequence Xaa¹-Xaa²-Xaa³-Xaa⁴ (SEQ ID NO: 19), and
(i) Xaa¹ is an amino acid residue selected from: Ala, Gly, Ser, His, and Leu;
(ii) Xaa² is any amino acid residue or absent;
(iii) Xaa³ is any amino acid residue or absent; and
(iv) Xaa⁴ consists of 3 or more His residues.

6. The N-terminally acylated protein of claim 1, wherein:
(A) X comprises the amino acid sequence Gly-Xaa¹-Xaa²-Xaa³ (SEQ ID NO: 4), and
(i) Xaa¹ is any amino acid residue or absent;
(ii) Xaa² is an amino acid residue or absent; and
(iii) Xaa³ consists of 3 or more His residues;
or
(B) X comprises the amino acid sequence Gly-Xaa¹, and
(i) Xaa¹ is an amino acid residue selected from Gly, Ala, and Ser.

7. The N-terminally acylated protein of claim 1, wherein:
(A) X comprises the amino acid sequence Xaa¹-Xaa², and
(i) Xaa¹ is amino acid residue Gly or absent; and
(ii) Xaa² consists of 1 or more His residues; or
(B) X comprises the amino acid sequence Gly-Xaa¹-Xaa², and
(i) Xaa¹ is an amino acid residue selected from Ser, Gly, Ala, Tyr, Leu, and Arg, or is absent; and
(ii) Xaa² consists of 2 or more His residues;
or
(C) X comprises the amino acid sequence Gly-Xaa¹-Xaa², and
(i) Xaa¹ is Gly, Ser or Ala; and
(ii) Xaa² is:
(SEQ ID NO: 10)
Thr-Tyr-Ser-Asp-His,
(SEQ ID NO: 12)
Thr-Tyr-Ser-Cys-His,
(SEQ ID NO: 11)
Thr-Tyr-Ser-Ala-His,
(SEQ ID NO: 13)
Lys-Trp-Ser-Lys-Arg,
or
(SEQ ID NO: 14)
Ser-Gly-Ser-Lys.

8. A composition comprising the N-terminally acylated protein of claim 1, and a metal cation.

9. The composition of claim 8, wherein:
(i) the metal cation is from the d-block elements; and/or
(ii) the composition further comprises a diol-ester formed between a lactone-derived diol and an acid at the N-terminus of the N-terminally acylated protein.

10. A method for site-specifically modifying a protein, comprising contacting a protein with a handle-substituted carbohydrate lactone.

11. The method of claim 10, wherein the protein is site-specifically modified at the N-terminus and the handle-substituted carbohydrate lactone is a compound according to formula (V): 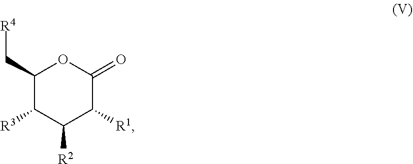
wherein:
(i) R¹, R² and R³ are each independently hydrogen, a hydroxyl, a methyl, or an azide,
(ii) R⁴ is hydrogen, a hydroxyl, a methyl, an azide, or a carboxyl,
(iii) at least one of R¹, R², R³ and R⁴ is an azide, and
(iv) one or none of R¹, R², R³ and R⁴ is a methyl.

12. The method of claim 10, wherein the method further comprises contacting the resulting site-specifically modified protein with a compound comprising a phosphine group, a phosphine derivative, alkene group, alkyne group, strained alkyne group, OCT, MOFO, DIFO, DIFO2, DIFO3, DIMAC, DIBO, DIBAC, BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group.

13. A protein obtained or obtainable through the method of claim 10.

14. (canceled)

15. A method for identifying an acylated protein, comprising running a sample suspected of containing the acylated protein on a diol-interacting, boron-containing acrylamide gel, wherein the acylated protein is the N-terminally acylated protein according to a claim 1.

16. A method for purifying the N-terminally acylated protein according to claim 1, the method comprising:
(1) binding a sample suspected of comprising the N-terminally acylated protein onto a solid support comprising an immobilized diol-ester forming agent; and
(2) eluting the protein.

17. A kit comprising:
(i) a protein; and
(ii) a handle-substituted carbohydrate lactone.

18. The kit of claim 17, wherein the kit further comprises a compound comprising a phosphine group, a phosphine derivative, alkene group, alkyne group, strained alkyne group, OCT, MOFO, DIFO, DIFO2, DIFO3, DIMAC, DIBO, DIBAC, BARAC, BCN, Sondheimer diyene, TMDIBO, S-DIBO, COMBO, PYRROC, TMTH, DIFBO, ALO, thioalkyne group, keto-DIBO, strained olefin, or oxanorbornadiene group.

19. The N-terminally acylated protein of claim 1, wherein the N-terminal amino acid residue is a Gly, Ala, Ser or His residue.

20. The composition of claim 9, wherein the metal cation is a divalent Zn, Ni, or Cu cation and/or the diol-ester is a borate-, or boronic-/boronate ester.


SEQUENCE LISTING (93 sequences)

SEQ ID NO: 1 - protein, 10 aa, Artificial Sequence
  Tag
MGSSHHHHHH

SEQ ID NO: 2 - protein, 7 aa, Artificial Sequence
  Tag
GHHHHHH

SEQ ID NO: 3 - protein, 9 aa, Artificial Sequence
  Tag
GSSHHHHHH

SEQ ID NO: 4 - protein, 4 aa, Artificial Sequence
  Tag
  VARIANT (2)..(2): An amino acid residue or absent
  VARIANT (3)..(3): An amino acid residue or absent
  VARIANT (4)..(4): 3 or more His residues
GXXX

SEQ ID NO: 5 - protein, 7 aa, Artificial Sequence
  Tag
  VARIANT (2)..(2): Gly, Ser or Ala
GXTYSDH

SEQ ID NO: 6 - protein, 7 aa, Artificial Sequence
  Tag
  VARIANT (2)..(2): Gly, Ser or Ala
GXTYSAH

SEQ ID NO: 7 - protein, 7 aa, Artificial Sequence
  Tag
  VARIANT (2)..(2): Gly, Ser or Ala
GXTYSCH

SEQ ID NO: 8 - protein, 7 aa, Artificial Sequence
  Tag
  VARIANT (2)..(2): Gly, Ser or Ala
GXKWSKR

SEQ ID NO: 9 - protein, 6 aa, Artificial Sequence
  Tag
  VARIANT (2)..(2): Gly, Ser or Ala
GXSGSK

SEQ ID NO: 10 - protein, 5 aa, Artificial Sequence
  Tag
TYSDH

SEQ ID NO: 11 - protein, 5 aa, Artificial Sequence
  Tag
TYSAH

SEQ ID NO: 12 - protein, 5 aa, Artificial Sequence
  Tag
TYSCH

SEQ ID NO: 13 - protein, 5 aa, Artificial Sequence
  Tag
KWSKR

SEQ ID NO: 14 - protein, 4 aa, Artificial Sequence
  Tag
SGSK

SEQ ID NO: 15 - protein, 7 aa, Artificial Sequence
  Tag
GGTYSDH

SEQ ID NO: 16 - protein, 7 aa, Artificial Sequence
  Tag
GGTYSCH

SEQ ID NO: 17 - protein, 7 aa, Artificial Sequence
  Tag
GGKWSKR

SEQ ID NO: 18 - protein, 6 aa, Artificial Sequence
  Tag
GASGSK

SEQ ID NO: 19 - protein, 9 aa, Artificial Sequence
  Tag
  VARIANT (1)..(1): Ala, Gly, Ser, His or Leu
  VARIANT (2)..(2): An amino acid residue or absent
  VARIANT (3)..(3): An amino acid residue or absent
  VARIANT (7)..(9): X is His or absent
XXXHHHXXX

SEQ ID NO: 20 - protein, 4 aa, Artificial Sequence
  Tag
AHHH

SEQ ID NO: 21 - protein, 4 aa, Artificial Sequence
  Tag
GAAH

SEQ ID NO: 22 - protein, 4 aa, Artificial Sequence
  Tag
GASH

SEQ ID NO: 23 - protein, 4 aa, Artificial Sequence
  Tag
GHHH

SEQ ID NO: 24 - protein, 4 aa, Artificial Sequence
  Tag
GSAH

SEQ ID NO: 25 - protein, 4 aa, Artificial Sequence
  Tag
GSSH

SEQ ID NO: 26 - protein, 4 aa, Artificial Sequence
  Tag
SSYH

SEQ ID NO: 27 - protein, 4 aa, Artificial Sequence
  Tag
SYYH

SEQ ID NO: 28 - protein, 4 aa, Artificial Sequence
  Tag
VHHH

SEQ ID NO: 29 - protein, 5 aa, Artificial Sequence
  Tag
GAPTL

SEQ ID NO: 30 - protein, 5 aa, Artificial Sequence
  Tag
GHHHH

SEQ ID NO: 31 - protein, 6 aa, Artificial Sequence
  Tag
GHHHHH

SEQ ID NO: 32 - protein, 6 aa, Artificial Sequence
  Tag
LRFKFY

SEQ ID NO: 33 - protein, 6 aa, Artificial Sequence
  Tag
HHHHHH

SEQ ID NO: 34 - protein, 7 aa, Artificial Sequence
  Tag
GASGSKG

SEQ ID NO: 35 - protein, 7 aa, Artificial Sequence
  Tag
GLRFKFY

SEQ ID NO: 36 - protein, 7 aa, Artificial Sequence
  Tag
HLRFKFY

SEQ ID NO: 37 - protein, 7 aa, Artificial Sequence
  Tag
KHHHHHH

SEQ ID NO: 38 - protein, 8 aa, Artificial Sequence
  Tag
GHLRFKFY

SEQ ID NO: 39 - protein, 8 aa, Artificial Sequence
  Tag
GSLRFKFY

SEQ ID NO: 40 - protein, 8 aa, Artificial Sequence
  Tag
GSHHHHHH

SEQ ID NO: 41 - protein, 9 aa, Artificial Sequence
  Tag
GSHLRFKFY

SEQ ID NO: 42 - protein, 9 aa, Artificial Sequence
  Tag
RGSHHHHHH

SEQ ID NO: 43 - protein, 9 aa, Artificial Sequence
  Tag
SYYHHHHHH

SEQ ID NO: 44 - protein, 4 aa, Artificial Sequence
  Tag
GHLR

SEQ ID NO: 45 - protein, 4 aa, Artificial Sequence
  Tag
GLRF

SEQ ID NO: 46 - protein, 4 aa, Artificial Sequence
  Tag
GAPT

SEQ ID NO: 47 - protein, 4 aa, Artificial Sequence
  Tag
GASG

SEQ ID NO: 48 - protein, 4 aa, Artificial Sequence
  Tag
GGKW

SEQ ID NO: 49 - protein, 4 aa, Artificial Sequence
  Tag
GGTY

SEQ ID NO: 50 - protein, 4 aa, Artificial Sequence
  Tag
GSHL

SEQ ID NO: 51 - protein, 4 aa, Artificial Sequence
  Tag
GSLR

SEQ ID NO: 52 - protein, 5 aa, Artificial Sequence
  Tag
GASGS

SEQ ID NO: 53 - protein, 5 aa, Artificial Sequence
  Tag
GGKWS

SEQ ID NO: 54 - protein, 5 aa, Artificial Sequence
  Tag
GGTYS

SEQ ID NO: 55 - protein, 5 aa, Artificial Sequence
  Tag
GHLRF

SEQ ID NO: 56 - protein, 5 aa, Artificial Sequence
  Tag
GLRFK

SEQ ID NO: 57 - protein, 5 aa, Artificial Sequence
  Tag
GSHLR

SEQ ID NO: 58 - protein, 5 aa, Artificial Sequence
  Tag
GSLRF

SEQ ID NO: 59 - protein, 5 aa, Artificial Sequence
  Tag
GSSHH

SEQ ID NO: 60 - protein, 6 aa, Artificial Sequence
  Tag
GASGSK

SEQ ID NO: 61 - protein, 6 aa, Artificial Sequence
  Tag
GGKWSK

SEQ ID NO: 62 - protein, 6 aa, Artificial Sequence
  Tag
GGTYSD

SEQ ID NO: 63 - protein, 6 aa, Artificial Sequence
  Tag
GHLRFK

SEQ ID NO: 64 - protein, 6 aa, Artificial Sequence
  Tag
GLRFKF

SEQ ID NO: 65 - protein, 6 aa, Artificial Sequence
  Tag
GSHLRF

SEQ ID NO: 66 - protein, 6 aa, Artificial Sequence
  Tag
GSLRFK

SEQ ID NO: 67 - protein, 6 aa, Artificial Sequence
  Tag
GSSHHH

SEQ ID NO: 68 - protein, 7 aa, Artificial Sequence
  Tag
GHLRFKF

SEQ ID NO: 69 - protein, 7 aa, Artificial Sequence
  Tag
GSHLRFK

SEQ ID NO: 70 - protein, 7 aa, Artificial Sequence
  Tag
GSLRFKF

SEQ ID NO: 71 - protein, 7 aa, Artificial Sequence
  Tag
GSSHHHH

SEQ ID NO: 72 - protein, 8 aa, Artificial Sequence
  Tag
GSHLRFKF

SEQ ID NO: 73 - protein, 8 aa, Artificial Sequence
  Tag
GSSHHHHH

SEQ ID NO: 74 - protein, 4 aa, Artificial Sequence
  Tag
HLRF

SEQ ID NO: 75 - protein, 5 aa, Artificial Sequence
  Tag
HLRFK

SEQ ID NO: 76 - protein, 6 aa, Artificial Sequence
  Tag
HLRFKF

SEQ ID NO: 77 - protein, 4 aa, Artificial Sequence
  Tag
LRFK

SEQ ID NO: 78 - protein, 5 aa, Artificial Sequence
  Tag
LRFKF

SEQ ID NO: 79 - protein, 9 aa, Artificial Sequence
  Tag
  VARIANT (2)..(2): Ser, Gly or Ala
  VARIANT (3)..(3): Ser, Gly or Ala
  VARIANT (7)..(9): X is His or absent
GXXHHHXXX

SEQ ID NO: 80 - protein, 9 aa, Artificial Sequence
  Tag
  VARIANT (7)..(9): X is His or absent
GSSHHHXXX

SEQ ID NO: 81 - protein, 7 aa, Artificial Sequence
  Tag
GRGDSPC

SEQ ID NO: 82 - protein, 7 aa, Artificial Sequence
  Tag
AHHHHHH

SEQ ID NO: 83 - protein, 11 aa, Artificial Sequence
  Tag
GHHHHHHHHHH

SEQ ID NO: 84 - protein, 9 aa, Artificial Sequence
  Tag
GHHHHHHHH

SEQ ID NO: 85 - protein, 9 aa, Artificial Sequence
  Tag
GGSHHHHHH

SEQ ID NO: 86 - protein, 133 aa, Artificial Sequence
  Qbeta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 87 - protein, 201 aa, Artificial Sequence
  His-RBD
GHHHHHHTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG

SEQ ID NO: 88 - DNA, 20 bp, Artificial Sequence
  Primer
gaccccaaaatcagcgaaat

SEQ ID NO: 89 - DNA, 24 bp, Artificial Sequence
  Primer
tctggttactgccagttgaatctg

SEQ ID NO: 90 - DNA, 24 bp, Artificial Sequence
  Probe
accccgcattacgtttggtggacc

SEQ ID NO: 91 - protein, 194 aa, Artificial Sequence
  RBD
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG

SEQ ID NO: 92 - protein, 27 aa, Artificial Sequence
  His-tagged Beltide-1 derivative peptide PP6
GSSHHHHHHDWLKAFYDKVAEKLKEAF

SEQ ID NO: 93 - protein, 29 aa, Artificial Sequence
  His-tagged peptide
GSSHHHHHHGGTYSAHFGPLTWVAKPQGG